(19) United States
(12) Patent Application Publication
(10) Pub. No.: US 2020/0405913 A1
(43) Pub. Date: Dec. 31, 2020

(54) INJECTABLE CELL AND SCAFFOLD COMPOSITIONS

(71) Applicants: Deepak Jain, Winston-Salem, NC (US); Timothy A. Bertram, George Town, KY (US)

(72) Inventors: Deepak Jain, Winston-Salem, NC (US); Timothy A. Bertram, George Town, KY (US)

(21) Appl. No.: 16/498,630

(22) PCT Filed: Mar. 26, 2018
(86) PCT No.: PCT/US18/24353

§ 371 Date: Sep. 27, 2019

Related U.S. Application Data
(60) Provisional application No. 62/480,166, filed on Mar. 31, 2017.

(51) Int. Cl. A61L 27/38; A61L 27/52; A61L 27/54; A61L 27/36; A61L 27/24

(52) U.S. Cl. CPC A61L 27/3813; A61L 27/52; A61L 27/24; A61L 27/3604; A61L 27/54

(57) ABSTRACT

Provided herein are, inter alia, therapeutic formulations containing active agents, such as bioactive cell populations, and methods of making and using the same.

### CROSS-REFERENCE TO RELATED APPLICATIONS

This application claims benefit of priority to U.S. Provisional Patent Application No. 62/480,166, filed Mar. 31, 2017, the entire content of which is incorporated by reference in its entirety.

### TECHNICAL FIELD

The present invention relates to, inter alia, cells, compositions, and methods for treating kidney disease.

### BACKGROUND

Chronic Kidney Disease (CKD) affects over 19 million people in the United States and is frequently a consequence of metabolic disorders involving obesity, diabetes, and hypertension (United States Renal Data System: Costs of CKD and ESRD. ed. Bethesda, Md., National Institutes of Health, National Institute of Diabetes and Digestive and Kidney Diseases, 2007 pp 223-238)—three diseases that are also on the rise worldwide. Obesity, hypertension, and poor glycemic control have all been shown to be independent risk factors for kidney damage, causing glomerular and tubular lesions and leading to proteinuria and other systemically-detectable alterations in renal filtration function (Aboushwareb, et al., World J Urol, 26: 295-300, 2008; Amann, K. et al., Nephrol Dial Transplant, 13: 1958-66, 1998).

Traditionally, clinical approaches to the treatment of chronic renal failure involve dialysis and kidney transplantation for restoration of renal filtration and urine production, and the systemic delivery of recombinant EPO or EPO analogs to restore erythroid mass. Dialysis offers survival benefit to patients in mid-to-late stage renal failure, but causes significant quality-of-life issues. Kidney transplant is a highly desired (and often the only) option for patients in the later stages of renal failure, but the supply of high-quality donor kidneys does not meet the demand of the renal failure population. Bolus dosing with recombinant EPO to treat anemia has now been associated with serious downstream health risks, leading to black box warnings from the FDA for the drug, and necessitating further investigation into alternative treatments to restore erythroid homeostasis in this population.

More recently, new treatment paradigms involving tissue engineering applications have been described that provide substantial and durable augmentation of kidney functions, slow progression of disease and improve quality of life in this patient population. Isolated, bioactive renal cells represent a candidate cell-based regenerative therapy for the treatment of chronic kidney disease. (Presnell et al. WO/2010/056328; Ilagan et al. PCT/US2011/036347). However, such cell-based therapies require sustained, physiologically relevant bioactivity to be maintained ex vivo and in the absence of standard cell culture environments. Product potency may be lost upon packaging of bioactive cells as cell-based therapeutic products without a biologically supportive formulation or carrier. Thus, there exists a need for therapeutic formulations that are suitable for delivery of bioactive agents, such as for example, bioactive cells for tissue engineering and regenerative medicine applications, to subjects in need. Formulation of isolated bioactive renal and/or non-renal cells into a neo-kidney augment (NKA) may provide enhanced stability of the cells, thus extending product shelf life, improving stability during transport and during delivery into the target organ or construct for clinical applications.

### BRIEF SUMMARY

The present disclosure relates generally to, inter alia, a combination regenerative construct for regeneration, repair and/or rescue of renal structure and/or function composed of biologically active renal and/or non-renal cell compositions complexed with a matrix, gel or scaffold that provides a supportive, three dimensional environment for the bioactive cell population, facilitating the extended biological potency of the cellular fraction as a therapeutic product for amelioration of renal disease.

In an aspect, provided herein is an injectable formulation. In certain embodiments, the formulation includes a) a temperature-sensitive cell-stabilizing biomaterial, and b) a bioactive renal cell (BRC) population. In certain embodiments, the temperature-sensitive cell-stabilizing biomaterial is a hydrogel that (i) maintains a substantially solid state at about 8° C. or below, wherein the substantially solid state is a gel state, (ii) maintains a substantially liquid state at about ambient temperature or above, and (iii) has a solid-to-liquid transitional state between about 8° C. and about ambient temperature or above. In certain embodiments, the hydrogel comprises an extracellular matrix protein of recombinant origin, is derived from extracellular matrix sourced from kidney or another tissue or organ, or comprises gelatin.

In certain embodiments, the gelatin is derived from Type I, alpha I collagen. In certain embodiments, the BRC (e.g., a selected renal cell population) is coated with, deposited on, embedded in, attached to, seeded, or entrapped in the biomaterial. In certain embodiments, the biomaterial is configured as porous foam, gel, liquid, beads, or solids.

In certain embodiments, the gelatin is derived from porcine Type I, alpha I collagen or recombinant human Type I, alpha I collagen.

In certain embodiments, the BRC is a selected renal cell (SRC) population. In certain embodiments, the BRC or SRC population contains a greater percentage of one or more cell populations and lacks, or is deficient in, one or more other cell populations, as compared to a starting renal cell population. In certain embodiments, the BRC or SRC population is enriched for tubular renal cells. In certain embodiments, the BRC or SRC population exhibits a cell morphology indicative of tubular renal cells. In certain embodiments, the BRC or SRC population is characterized by phenotypic expression of one or more tubular epithelial cell markers. In certain embodiments, the one or more tubular epithelial cell markers comprise CK18 and/or GGT1. In certain embodiments, the BRC or SRC population exhibits cell growth kinetics indicative of viable and metabolically active renal cells. In certain embodiments, the BRC or SRC population is characterized by phenotypic expression of one or more viability and/or functionality markers. In certain embodiments, the one or more viability and/or functionality markers comprise VEGF and/or KIM-1. In certain embodiments, the BRC or SRC population is characterized by LAP and/or GGT enzymatic activity.

In certain embodiments, the gelatin is present in the formulation at about 0.5% to about 1% (w/v). In certain embodiments, the gelatin is present in the formulation at about 0.8% to about 0.9% (w/v). In certain embodiments, the formulation further comprises a cell viability agent. In certain embodiments, the cell viability agent comprises an agent selected from the group consisting of an antioxidant, an oxygen carrier, a growth factor, a cell-stabilizing factor, an immunomodulatory factor, a cell recruitment factor, a cell attachment factor, an anti-inflammatory agent, an immunosuppressant, an angiogenic factor, and a wound healing factor. In certain embodiments, the cell viability agent is selected from the group consisting of human plasma, human platelet lysate, bovine fetal plasma or bovine pituitary extract.

In certain embodiments, a formulation provided herein comprises products secreted by a renal cell population.

In an aspect, provided herein is an implantable formulation. In certain embodiments, The formulation includes a) a decellularized kidney of human or animal origin or a cell-stabilizing biomaterial that has been structurally engineered through three dimensional bioprinting, and b) a BRC population.

In an aspect, provided herein is an injectable formulation. In certain embodiments, the formulation includes a) a biomaterial comprising about 0.88% (w/v) gelatin, wherein the gelatin is derived from Type I, alpha I collagen, and b) a composition comprising an SRC population. In certain embodiments, the SRC population comprises an enriched population of tubular renal cells and having a density greater than about 1.04 g/mL.

In an aspect, provided herein is a method for preparing an injectable formulation comprising a temperature-sensitive cell-stabilizing biomaterial and an admixture of bioactive renal cells, the method comprising the steps of: i) obtaining renal cortical tissue from the donor/recipient; ii) isolating renal cells from the kidney tissue by enzymatic digestion and expanding the renal cells using standard cell culture techniques; iii) subjecting the harvested renal cells to separation across a density boundary or density interface or single step discontinuous gradient to obtain an SRC population; and iv) reconstituting the bioactive cells with a gelatin-based hydrogel biomaterial, wherein the gelatin is derived from Type I, alpha I collagen.

In certain embodiments, the selected renal cells comprise an enriched population of tubular renal cells and having a density greater than about 1.04 g/mL.

In certain embodiments, the harvested renal cells are exposed to hypoxic culture conditions prior to separation across a density boundary or density interface or continuous or discontinuous single step or multistep density gradient.

In certain embodiments, the renal cells are enriched for tubular renal cells.

In certain embodiments, the method further comprises monitoring the cell morphology of the renal cells during cell expansion.

In certain embodiments, the renal cells exhibit a cell morphology indicative of tubular renal cells.

In certain embodiments, the method further comprises monitoring the cell growth kinetics of the renal cells at each cell passage. In certain embodiments, the method further comprises monitoring renal cell counts and viability using a reagent for evaluation of metabolic activity. In certain embodiments, the method further comprises monitoring the renal cells for phenotypic expression of one or more viability and/or functionality markers.

In certain embodiments, the one or more viability and/or functionality markers comprise VEGF and/or KIM-1.

In certain embodiments, the method further comprises monitoring the renal cells for phenotypic expression of one or more tubular epithelial cell markers. In certain embodiments, the one or more tubular epithelial cell markers comprise CK18 and/or GGT1.

In certain embodiments, the method further comprises monitoring renal cell functionality by gene expression profiling or measurement of enzymatic activities. In certain embodiments, the measured enzymatic activity is for LAP and/or GGT.

In certain embodiments, the renal cells are derived from an autologous or allogeneic kidney sample. In certain embodiments, the renal cells are derived from a non-autologous kidney sample. In certain embodiments, the sample is obtained by kidney biopsy.

In certain embodiments, the SRC are resuspended in a liquefied gelatin solution at 26-30° C. In certain embodiments, the SRC are resuspended in sufficient gelatin solution to achieve an SRC concentration of 100×10⁶ cells/ml.

In certain embodiments, the method further comprises rapidly cooling the SRC/gelatin solution to stabilize the biomaterial such that the SRC will remain suspended in the gel on storage.

In certain embodiments, the formulation is stored at a temperature range of 2−8° C.

In certain embodiments, the method comprises the addition of a cell viability agent. In certain embodiments, the cell viability agent comprises an agent selected from the group consisting of an antioxidant, an oxygen carrier, a growth factor, a cell-stabilizing factor, an immunomodulatory factor, a cell recruitment factor, a cell attachment factor, an anti-inflammatory agent, an immunosuppressant, an angiogenic factor, and a wound healing factor. In certain embodiments, the cell viability agent is selected from the group consisting of human plasma, human platelet lysate, bovine fetal plasma or bovine pituitary extract.

In an aspect, provided herein is method of treating kidney disease in a subject, the method comprising injecting a formulation, composition, or cell population disclosed herein into the subject. In certain embodiments, the formulation, composition, for cell population is injected through a 18 to 30 gauge needle. In certain embodiments, the formulation, composition, for cell population is injected through a needle that is smaller than 20 gauge. In certain embodiments, the formulation, composition, for cell population is injected through a needle that is smaller than 21 gauge. In certain embodiments, the formulation, composition, for cell population is injected through a needle that is smaller than 22 gauge. In certain embodiments, the formulation, composition, for cell population is injected through a needle that is smaller than 23 gauge. In certain embodiments, the formulation, composition, for cell population is injected through a needle that is smaller than 24 gauge. In certain embodiments, the formulation, composition, for cell population is injected through a needle that is smaller than 25 gauge. In certain embodiments, the formulation, composition, for cell population is injected through a needle that is smaller than 26 gauge. In certain embodiments, the formulation, composition, for cell population is injected through a needle that is smaller than 27 gauge. In certain embodiments, the formulation, composition, for cell population is injected through a needle that is smaller than 28 gauge. In certain embodiments, the formulation, composition, for cell population is injected through a needle that is smaller than 29 gauge. In certain embodiments, the formulation, composition, for cell population is injected through a needle that is about 20 gauge. In certain embodiments, the formulation, composition, for cell population is injected through a needle that is about 21 gauge. In certain embodiments, the formulation, composition, for cell population is injected through a needle that is about 22 gauge. In certain embodiments, the formulation, composition, for cell population is injected through a needle that is about 23 gauge. In certain embodiments, the formulation, composition, for cell population is injected through a needle that is about 24 gauge. In certain embodiments, the formulation, composition, for cell population is injected through a needle that is about 25 gauge. In certain embodiments, the formulation, composition, for cell population is injected through a needle that is about 26 gauge. In certain embodiments, the formulation, composition, for cell population is injected through a needle that is about 27 gauge. In certain embodiments, the formulation, composition, for cell population is injected through a needle that is about 28 gauge. In certain embodiments, the formulation, composition, for cell population is injected through a needle that is about 29 gauge.

In one aspect, the present disclosure concerns an injectable formulation comprising a temperature-sensitive cell-stabilizing biomaterial and a composition comprising a bioactive renal cell population (BRC). In certain embodiments, the bioactive renal cell population of the injectable formulation is a selected renal cell (SRC) population obtained after separation of the expanded renal cells across a density boundary, barrier, or interface (e.g., single-step discontinuous density gradient separation). In embodiments, the SRC may exhibit a buoyant density greater than approximately 1.04 g/mL. In embodiments, the SRC may exhibit a buoyant density greater than approximately 1.0419 g/mL. In embodiments, the SRC may exhibit a buoyant density greater than approximately 1.045 g/mL. In certain embodiments, the BRC or SRC the injectable formulation contains a greater percentage of one or more cell populations and lacks or is deficient in one or more other cell populations, as compared to a starting kidney cell population. In certain embodiments, the BRC or SRC may be enriched for tubular renal cells. The BRC or SRC may exhibit a cell morphology indicative of tubular renal cells and/or may be characterized by phenotypic expression of one or more tubular epithelial cell markers. In a particular embodiment, the one or more tubular epithelial cell markers comprise CK18 and/or GGT1.

In certain embodiments, the BRC or SRC of the injectable formulation may exhibit cell growth kinetics indicative of viable and metabolically active renal cells. In certain embodiments, the BRC or SRC are characterized by phenotypic expression of one or more viability and/or functionality markers. In a particular embodiment, the one or more viability and/or functionality markers comprise VEGF and/or KIM-1. In certain embodiments of the injectable formulation, the BRC or SRC functionality is further established by gene expression profiling or measurement of enzymatic activities. The measured enzymatic activity may be for LAP and/or GGT. In some embodiments, the BRC or SRC of the injectable formulation is derived from an autologous or allogeneic kidney sample. In some other embodiments, the BRC or SRC is derived from a non-autologous kidney sample. The sample may be obtained by kidney biopsy.

In some embodiments, the temperature-sensitive cell-stabilizing biomaterial of the injectable formulation maintains a substantially solid state at about 8° C. or below, and a substantially liquid state at about ambient temperature or above. In certain embodiments, the biomaterial may comprise a solid-to-liquid transitional state between about 8° C. and about ambient temperature or above. The substantially solid state may be a gel state. In certain embodiments, the biomaterial comprises a gelatin-based hydrogel. The gelatin may be present in the formulation at about 0.5% to about 1% (w/v). In specific embodiments, the gelatin is present in the formulation at about 0.8% to about 0.9% (w/v).

In one or more embodiments, the bioactive cells of the injectable formulation are substantially uniformly dispersed throughout the volume of the cell-stabilizing biomaterial. In some embodiments, the injectable formulation further comprises a cell viability agent. The cell viability agent may comprise an agent selected from the group consisting of an antioxidant, an oxygen carrier, a growth factor, a cell-stabilizing factor, an immunomodulatory factor, a cell recruitment factor, a cell attachment factor, an anti-inflammatory agent, an immunosuppressant, an angiogenic factor, and a wound healing factor. In specific embodiments, the cell viability agent may be selected from the group consisting of human plasma, human platelet lysate, bovine fetal plasma or bovine pituitary extract. In certain embodiments, the injectable formulation comprises a biomaterial comprising about 0.88% (w/v) gelatin, and a composition comprising a bioactive renal cell population (BRC), wherein the BRC comprise an enriched population of tubular renal cells and having a density greater than about 1.04 g/mL. In certain embodiments, the injectable formulation comprises a biomaterial comprising about 0.88% (w/v) gelatin, and a composition comprising a bioactive renal cell population (BRC), wherein the BRC comprise an enriched population of tubular renal cells and having a density greater than about 1.0419 g/mL or about 1.045 g/mL.

In another aspect, the present disclosure concerns a method for preparing an injectable formulation comprising a temperature-sensitive cell-stabilizing biomaterial and an admixture of bioactive renal cells, the method comprising the steps of: i) obtaining renal cortical tissue from the donor/recipient; ii) isolating renal cells from the kidney tissue by enzymatic digestion and expanding the renal cells using standard cell culture techniques; iii) subjecting the harvested renal cells to separation by centrifugation across a density boundary, barrier, or interface to obtain Selected Renal Cells (SRC); and iv) reconstituting the bioactive cells with a gelatin-based hydrogel biomaterial. In embodiments, the selected renal cells may comprise an enriched population of tubular renal cells and having a density greater than about 1.04 g/mL. The selected renal cells may comprise an enriched population of tubular renal cells and having a density greater than about 1.0419 g/mL or 1.045 g/mL. In certain embodiments, the harvested renal cells are exposed to hypoxic culture conditions prior to separation by centrifugation across a density boundary, barrier, or interface. In certain embodiments, the renal cells are enriched for tubular renal cells.

In certain embodiments, the method for preparing the injectable formulation further comprises monitoring the cell morphology of the renal cells during cell expansion. The selected renal cells exhibit a cell morphology indicative of tubular renal cells. In certain embodiments, the method comprises monitoring the cell growth kinetics of the renal cells at each cell passage. In yet another embodiment, the method comprises monitoring renal cell counts and viability using a reagent for evaluation of metabolic activity. In some embodiments, the method comprises monitoring the renal cells for phenotypic expression of one or more viability and/or functionality markers. The one or more viability and/or functionality markers may comprise VEGF and/or KIM-1. In still other embodiments, the method includes monitoring the renal cells for phenotypic expression of one or more tubular epithelial cell markers. The one or more tubular epithelial cell markers may comprise CK18 and/or GGT1. The method may also comprise monitoring renal cell functionality by gene expression profiling or measurement of enzymatic activities. The measured enzymatic activity may include LAP and/or GGT activity.

In some embodiments, the renal cells used in the method for preparing the injectable formulation are derived from an autologous or allogeneic kidney sample. In certain embodiments, the renal cells are derived from a non-autologous kidney sample. The kidney sample may be obtained by kidney biopsy.

In certain embodiments, the SRC used in the method for preparing the injectable formulation are resuspended in a liquefied gelatin solution at 26-30° C. The SRC may be resuspended in sufficient gelatin solution to achieve an SRC concentration of 100×10⁶ cells/ml. In certain embodiments, the method comprises rapidly cooling the SRC/gelatin solution to stabilize the biomaterial such that the SRC will remain suspended in the gel on storage. The formulation may be stored at a temperature range of 2-8° C.

In yet another embodiment, the method for preparing the injectable formulation comprises the addition of a cell viability agent. The cell viability agent may be an agent selected from the group consisting of an antioxidant, an oxygen carrier, a growth factor, a cell-stabilizing factor, an immunomodulatory factor, a cell recruitment factor, a cell attachment factor, an anti-inflammatory agent, an immunosuppressant, an angiogenic factor, and a wound healing factor. In certain embodiments, the cell viability agent is selected from the group consisting of human plasma, human platelet lysate, bovine fetal plasma or bovine pituitary extract.

Additional aspects and embodiments are disclosed below. Each embodiment disclosed herein is contemplated as being applicable to each of the other disclosed embodiments. Thus, all combinations of the various elements described herein are within the scope of the present disclosure.

### BRIEF DESCRIPTION OF THE DRAWINGS

FIG. 1: Human Renal Cell Morphology in Culture.

FIG. 2: SRC Banding by centrifugation across a density boundary.

FIG. 3: Gelatin Solution Temperature Profile for Gelation.

FIG. 4: Rotation Time During NKA Gelation.

FIG. 5: Expression of Renal Cell Markers in Human SRC Populations.

FIG. 6: Enzymatic Activity of Human SRC.

FIG. 7: SRC Settling over a 3 Day Hold Time at Cold Temperature.

FIG. 8: SRC Distribution in NKA using Confocal Microscopy.

FIG. 9: NKA Sampling Across the Syringe.

FIG. 10: Total Live Cell Distribution in NKA Across the Syringe.

FIG. 11: SRC Dispersion in NKA after Formulation.

FIG. 12: SRC Dispersion in NKA Across Syringe after 3 Day Hold.

FIG. 13: Stability of NKA Viability by Trypan Blue on Cold Storage.

FIG. 14: Stability of NKA Phenotype by CK18 on Cold Storage.

FIG. 15: Stability of NKA Phenotype by GGT1 on Cold Storage.

FIG. 16: Stability of NKA by PrestoBlue Metabolism on Cold Storage.

FIG. 17: Stability of NKA Function by VEGF on Cold Storage.

FIG. 18: Compatibility of Delivery Cannula with NKA.

FIG. 19: Illustration of NKA Delivery and Implantation.

FIG. 20: Flow diagram of a non-limiting example of an overall NKA manufacturing process.

FIG. 21A-D: Flow diagrams providing further details of the non-limiting example process depicted in FIG. 20.

### DETAILED DESCRIPTION

Reference will now be made in detail to certain embodiments of the present invention. While aspects of the present disclosure will be described in conjunction with the embodiments, it will be understood that they are not intended to limit the invention to those embodiments. On the contrary, the present invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described.

All references cited throughout the disclosure are expressly incorporated by reference herein in their entirety. In the event that one or more of the incorporated literature, patents, and similar materials differs from or contradicts this application, including but not limited to defined terms, term usage, described techniques, or the like, this application controls.

### 1. Definitions

Unless defined otherwise, technical and scientific terms used herein have the same meaning as commonly understood by one of ordinary skill in the art to which this invention belongs. Principles of Tissue Engineering, 3rd Ed. (Edited by R Lanza, R Langer, & J Vacanti), 2007 provides one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The term “cell population” as used herein refers to a number of cells obtained by isolation directly from a suitable tissue source, usually from a mammal. For example, a cell population may comprise populations of kidney cells, and admixtures thereof. The isolated cell population may be subsequently cultured in vitro. Those of ordinary skill in the art will appreciate that various methods for isolating and culturing cell populations for use with the present disclosure and various numbers of cells in a cell population that are suitable for use in the present disclosure. A cell population may be an unfractionated, heterogeneous cell population or an enriched homogeneous cell population derived from an organ or tissue, e.g., the kidney. For example, a heterogeneous cell population may be isolated from a tissue biopsy or from whole organ tissue. Alternatively, the heterogeneous cell population may be derived from in vitro cultures of mammalian cells, established from tissue biopsies or whole organ tissue. An unfractionated heterogeneous cell population may also be referred to as a non-enriched cell population. In certain embodiments, the cell populations contain bioactive cells. Homogenous cell populations comprise a greater proportion of cells of the same cell type, sharing a common phenotype, or having similar physical properties, as compared to an unfractionated, heterogeneous cell population. For example, a homogeneous cell population may be isolated, extracted, or enriched from heterogeneous kidney cell population. In certain embodiments, a homogeneous cell population is obtained as a cell fraction using separation by centrifugation across a density boundary, barrier, or interface of a heterogeneous cell suspension. In certain embodiments, a homogeneous cell population is obtained as a cell fraction using continuous or discontinuous (single step or multi-step) density gradient separation of a heterogeneous cell suspension. In certain embodiments, a homogenous or heterogeneous cell population sourced from the kidney is admixed with a homogenous or heterogeneous cell population sourced from a tissue or organ other than the kidney, without further limitation.

As used herein, the term “bioactive” means “possessing biological activity,” such as a pharmacological or a therapeutic activity. In certain embodiments, the bioactivity is enhancement of renal function and/or effect on renal homeostasis. In certain embodiments, the biological activity is, without limitation, analgesic; antiviral; anti-inflammatory; antineoplastic; immune stimulating; immune modulating; enhancement of cell viability, antioxidation, oxygen carrier, cell recruitment, cell attachment, immunosuppressant, angiogenesis, wound healing activity, mobilization of host stem or progenitor cells, cellular proliferation, stimulation of cell migration to injury sites, amelioration of cell and tissue fibrosis, interference with the epithelial-mesenchymal signaling cascade, secretion of cytokines, growth factors, proteins, nucleic acids, exosomes, microvesicles or any combination thereof.

The term “bioactive renal cells” or “BRCs” as used herein refers to renal cells having one or more of the following properties when administered into the kidney of a subject: capability to reduce (e.g., slow or halt) the worsening or progression of chronic kidney disease or a symptom thereof, capability to enhance renal function, capability to affect (improve) renal homeostasis, and capability to promote healing, repair and/or regeneration of renal tissue or kidney. In embodiments, these cells may include functional tubular cells (e.g., based on improvements in creatinine excretion and protein retention), glomerular cells (e.g., based on improvement in protein retention), vascular cells and other cells of the corticomedullary junction. In embodiments, BRCs are obtained from isolation and expansion of renal cells from kidney tissue. In embodiments, BRCs are obtained from isolation and expansion of renal cells from kidney tissue using methods that select for bioactive cells. In embodiments, the BRCs have a regenerative effect on the kidney. In embodiments, BRCs comprise, consist essentially of, or consist of selected renal cells (SRCs). In embodiments, BRCs are SRCs.

In embodiments, SRCs are cells obtained from isolation and expansion of renal cells from a suitable renal tissue source, wherein the SRCs contain a greater percentage of one or more cell types and lacks or has a lower percentage of one or more other cell types, as compared to a starting kidney cell population. In embodiments, the SRCs contain an increased proportion of BRCs compared to a starting kidney cell population. In embodiments, an SRC population is an isolated population of kidney cells enriched for specific bioactive components and/or cell types and/or depleted of specific inactive and/or undesired components or cell types for use in the treatment of kidney disease, i.e., providing stabilization and/or improvement and/or regeneration of kidney function. SRCs provide superior therapeutic and regenerative outcomes as compared with the starting population. In embodiments, SRCs are obtained from the patient's renal cortical tissue via a kidney biopsy. In embodiments, SRCs are selected (e.g., by fluorescence-activated cell sorting or “FACS”) based on their expression of one or more markers. In embodiments, SRCs are depleted (e.g., by fluorescence-activated cell sorting or “FACS”) of one or more cell types based on the expression of one or more markers on the cell types. In embodiments, SRCs are selected from a population of bioactive renal cells. In embodiments, SRCs are selected by density gradient separation of expanded renal cells. In embodiments, SRCs are selected by separation of expanded renal cells by centrifugation across a density boundary, barrier, or interface, or single step discontinuous step gradient separation. In embodiments, SRCs are selected by continuous or discontinuous density gradient separation of expanded renal cells that have been cultured under hypoxic conditions. In embodiments, SRCs are selected by density gradient separation of expanded renal cells that have been cultured under hypoxic conditions for at least about 8, 12, 16, 20, or 24 hours. In embodiments, SRCs are selected by separation by centrifugation across a density boundary, barrier, or interface of expanded renal cells that have been cultured under hypoxic conditions. In embodiments, SRCs are selected by separation of expanded renal cells that have been cultured under hypoxic conditions for at least about 8, 12, 16, 20, or 24 hours by centrifugation across a density boundary, barrier, or interface (e.g., single-step discontinuous density gradient separation). In embodiments, SRCs are composed primarily of renal tubular cells. In embodiments, other parenchymal (e.g., vascular) and stromal (e.g., collecting duct) cells may be present in SRCs. In embodiments, less than about 10%, 9%, 8%, 7%, 6%, 5%, 4%, 3%, 2%, or 1% of the cells in a population of SRCs are vascular cells. In embodiments, less than about 10%, 9%, 8%, 7%, 6%, 5%, 4%, 3%, 2%, or 1% of the cells in a population of SRCs are collecting duct cells. In embodiments, less than about 10%, 9%, 8%, 7%, 6%, 5%, 4%, 3%, 2%, or 1% of the cells in a population of SRCs are vascular or collecting duct cells.

The term “native organ” shall mean the organ of a living subject. The subject may be healthy or unhealthy. An unhealthy subject may have a disease associated with that particular organ.

The term “native kidney” shall mean the kidney of a living subject. The subject may be healthy or unhealthy. An unhealthy subject may have a kidney disease.

The term “regenerative effect” shall mean an effect which provides a benefit to a native organ, such as the kidney. The effect may include, without limitation, a reduction in the degree of injury to a native organ or an improvement in, restoration of, or stabilization of a native organ function. Renal injury may be in the form of fibrosis, inflammation, glomerular hypertrophy, etc. and related to a disease associated with the native organ in the subject.

The term “admixture” as used herein refers to a combination of two or more isolated, enriched cell populations derived from an unfractionated, heterogeneous cell population. According to certain embodiments, the cell populations of the present disclosure are renal cell populations. In alternative embodiments, the cell populations may be admixtures of renal cell populations and non-renal cell populations, including, without limitation, mesenchymal stem cells and endothelial progenitor cells.

An “enriched” cell population or preparation refers to a cell population derived from a starting organ cell population (e.g., an unfractionated, heterogeneous cell population) that contains a greater percentage of a specific cell type than the percentage of that cell type in the starting population. For example, a starting kidney cell population can be enriched for a first, a second, a third, a fourth, a fifth, and so on, cell population of interest. As used herein, the terms “cell population”, “cell preparation” and “cell phenotype” are used interchangeably.

The term “hypoxic” culture conditions as used herein refers to culture conditions in which cells are subjected to a reduction in available oxygen levels in the culture system relative to standard culture conditions in which cells are cultured at atmospheric oxygen levels (about 21%). Non-hypoxic conditions are referred to herein as normal or normoxic culture conditions.

The term “oxygen-tunable” as used herein refers to the ability of cells to modulate gene expression (up or down) based on the amount of oxygen available to the cells.

The term “biomaterial” as used herein refers to a natural or synthetic biocompatible material that is suitable for introduction into living tissue supporting the selected bioactive cells in a viable state. A natural biomaterial is a material that is made by or originates from a living system. Synthetic biomaterials are materials which are not made by or do not originate directly from a living system, but are instead synthesized or composed by specific chemical procedures and protocols well known to those of ordinary skill in the art. The biomaterials disclosed herein may be a combination of natural and synthetic biocompatible materials. As used herein, biomaterials include, for example, polymeric matrices and scaffolds. Those of ordinary skill in the art will appreciate that the biomaterial(s) may be configured in various forms, for example, as porous foam, gels, liquids, beads, solids, and may comprise one or more natural or synthetic biocompatible materials. In certain embodiments, the biomaterial is the liquid form of a solution that is capable of becoming a hydrogel.

As used herein, biomaterials include, for example, extracellular matrix derived from an existing kidney of human or animal origin, wherein the native cell population has been eliminated through application of detergents and/or other chemical agents known to those of ordinary skill in the art. In certain embodiments, the biomaterial is a liquid form of a solution that is capable of becoming a hydrogel and is layered with or without certain cell populations by application of three-dimensional bioprinting methodologies known to those skilled in the art. In certain embodiments, the biomaterial is configured to mimic the three dimensional fractal organization of decellurized kidney.

The term “modified release” or the equivalent terms “controlled release”, “delayed release”, or “slow release” refer to formulations that release an active agent, such as bioactive cells, over time or at more than one point in time following administration to an individual. Modified release of an active agent, which can occur over a range of desired times, e.g., minutes, hours, days, weeks, or longer, depending upon the formulation, is in contrast to standard formulations in which substantially the entire dosage unit is available immediately after administration. For tissue engineering and regenerative medicine applications, preferred modified release formulations provide for the release of an active agent at multiple time points following local administration (e.g., administration of an active agent directly to a solid organ). For example, a modified release formulation of bioactive cells would provide an initial release of cells immediately at the time of administration and a later, second release of cells at a later time. The time delay for the second release of an active agent may be minutes, hours, or days after the initial administration. In general, the period of time for delay of release corresponds to the period of time that it takes for a biomaterial carrier of the active agent to lose it structural integrity. The delayed release of an active agent begins as soon as such integrity begins to degrade and is completed by the time integrity fails completely. Those of ordinary skill in the art will appreciate other suitable mechanisms of release.

The terms “construct” or “formulation” refer to one or more cell populations deposited on or in a surface of a scaffold or matrix made up of one or more synthetic or naturally-occurring biocompatible materials. The one or more cell populations may be coated with, deposited on, embedded in, attached to, seeded, or entrapped in a biomaterial made up of one or more synthetic or naturally-occurring biocompatible biomaterials, polymers, proteins, or peptides. In certain embodiments, the naturally occurring biomaterial is decellularized kidney of human or animal origin. In certain embodiments, the biomaterial has been structurally engineered through three dimensional bioprinting. The one or more cell populations may be combined with a biomaterial or scaffold or matrix in vitro or in vivo. The one or more biomaterials used to generate the construct or formulation may be selected to direct, facilitate, or permit dispersion and/or integration of the cellular components of the construct with the endogenous host tissue, or to direct, facilitate, or permit the survival, engraftment, tolerance, or functional performance of the cellular components of the construct or formulation. In certain embodiments, the one or more biocompatible materials used to form the scaffold/biomaterial is selected to direct, facilitate, or permit the formation of multicellular, three-dimensional, organization of at least one of the cell populations deposited thereon. In certain embodiments, the biomaterials direct the assembly of defined three dimensional cellular aggregrates or organoids that recapitulate aspects of native kidney tissue, including but not limited to organizational polarity. In certain embodiments, the biomaterials direct the assembly of defined tubular structures that recapitulate aspects of native kidney tissue, including lumens. In certain embodiments, the biomaterials promote or facilitate the secretion of proteins, nucleic acids and membrane-bound vesicles from the cell populations deposited herein. In general, the one or more biomaterials used to generate the construct may also be selected to mimic or recapitulate aspects of the specific three dimensional organization or environmental niche within native kidney or renal parenchyma representing the original biological environment from which these cell populations were derived. Recreation of the original biological niche from which these cell populations were sourced is believed to further promote or facilitate cell viability and potency.

The term “cellular aggregate” or “spheroid” refers to an aggregate or assembly of cells cultured to allow 3D growth as opposed to growth as a monolayer. It is noted that the term “spheroid” does not imply that the aggregate is a geometric sphere. The aggregate may be highly organized with a well defined morphology and polarity or it may be an unorganized mass; it may include a single cell type or more than one cell type. The cells may be primary isolates, or a permanent cell line, or a combination of the two. Included in this definition are organoids and organotypic cultures. In certain embodiments, the spheroids (e.g., cellular aggregates or organoids) are formed in a spinner flask. In certain embodiments, the spheroids (e.g., cellular aggregates or organoids) are formed in a 3-dimensional matrix.

The term “ambient temperature” refers to the temperature at which the formulations of the present disclosure will be administered to a subject. Generally, the ambient temperature is the temperature of a temperature-controlled environment. Ambient temperature ranges from about 18° C. to about 30° C. In certain embodiments, ambient temperature is about 18° C., about 19° C., about 20° C., about 21° C., about 22° C., about 23° C., about 24° C., about 25° C., about 26° C., about 27° C., about 28° C., about 29° C., or about 30° C.

The term “hydrogel” is used herein to refer to a substance formed when an organic polymer (natural or synthetic) is crosslinked via covalent, ionic, or hydrogen bonds to create a three-dimensional open-lattice structure which entraps water molecules to form a gel. Examples of materials which can be used to form a hydrogel include polysaccharides such as alginate, polyphosphazines, and polyacrylates, which are crosslinked ionically, or block copolymers such as Pluronics™ or Tetronics™, polyethylene oxide-polypropylene glycol block copolymers which are crosslinked by temperature or pH, respectively. The hydrogel used herein is preferably a biodegradable gelatin-based hydrogel.

The term “Neo-Kidney Augment (NKA)” refers to a bioactive cell formulation which is an injectable product composed of autologous, selected renal cells (SRC) formulated in a biomaterial comprised of a gelatin-based hydrogel.

The term “kidney disease” as used herein refers to disorders associated with any stage or degree of acute or chronic renal failure that results in a loss of the kidney's ability to perform the function of blood filtration and elimination of excess fluid, electrolytes, and wastes from the blood. Kidney disease may also include endocrine dysfunctions such as anemia (erythropoietin-deficiency), and mineral imbalance (Vitamin D deficiency). Kidney disease may originate in the kidney or may be secondary to a variety of conditions, including (but not limited to) heart failure, hypertension, diabetes, autoimmune disease, or liver disease. Kidney disease may be a condition of chronic renal failure that develops after an acute injury to the kidney. For example, injury to the kidney by ischemia and/or exposure to toxicants may cause acute renal failure; incomplete recovery after acute kidney injury may lead to the development of chronic renal failure.

The term “treatment” refers to both therapeutic treatment and prophylactic or preventative measures for kidney disease, tubular transport deficiency, or glomerular filtration deficiency wherein the object is to reverse, prevent or slow down (lessen) the targeted disorder. Those in need of treatment include those already having a kidney disease, tubular transport deficiency, or glomerular filtration deficiency as well as those prone to having a kidney disease, tubular transport deficiency, or glomerular filtration deficiency or those in whom the kidney disease, tubular transport deficiency, or glomerular filtration deficiency is to be prevented. The term “treatment” as used herein includes the stabilization and/or improvement of kidney function.

The term “in vivo contacting” as used herein refers to direct contact in vivo between products secreted by an enriched population of cells and a native organ. For example, products secreted by an enriched population of renal cells (or an admixture or construct containing renal cells/renal cell fractions) may in vivo contact a native kidney. The direct in vivo contacting may be paracrine, endocrine, or juxtacrine in nature. The products secreted may be a heterogeneous population of different products described herein.

The term “subject” shall mean any single human subject, including a patient, eligible for treatment, who is experiencing or has experienced one or more signs, symptoms, or other indicators of a kidney disease. Such subjects include without limitation subjects who are newly diagnosed or previously diagnosed and are now experiencing a recurrence or relapse, or are at risk for a kidney disease, no matter the cause. The subject may have been previously treated for a kidney disease, or not so treated.

The term “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “sample” or “patient sample” or “biological sample” shall generally mean any biological sample obtained from a subject or patient, body fluid, body tissue, cell line, tissue culture, or other source. The term includes tissue biopsies such as, for example, kidney biopsies. The term includes cultured cells such as, for example, cultured mammalian kidney cells. Methods for obtaining tissue biopsies and cultured cells from mammals are well known in the art. If the term “sample” is used alone, it shall still mean that the “sample” is a “biological sample” or “patient sample”, i.e., the terms are used interchangeably. The term “test sample” refers to a sample from a subject that has been treated by a method of the present disclosure. The test sample may originate from various sources in the mammalian subject including, without limitation, blood, semen, serum, urine, bone marrow, mucosa, tissue, etc.

The term “control” or “control sample” refers a negative or positive control in which a negative or positive result is expected to help correlate a result in the test sample. Controls that are suitable for the present disclosure include, without limitation, a sample known to exhibit indicators characteristic of normal kidney function, a sample obtained from a subject known not to have kidney disease, and a sample obtained from a subject known to have kidney disease. In addition, the control may be a sample obtained from a subject prior to being treated by a method of the present disclosure. An additional suitable control may be a test sample obtained from a subject known to have any type or stage of kidney disease, and a sample from a subject known not to have any type or stage of kidney disease. A control may be a normal healthy matched control. Those of skill in the art will appreciate other controls suitable for use in the present disclosure.

“Regeneration prognosis”, “regenerative prognosis”, or “prognostic for regeneration” generally refers to a forecast or prediction of the probable regenerative course or outcome of the administration or implantation of a cell population, admixture or construct described herein. For a regeneration prognosis, the forecast or prediction may be informed by one or more of the following: improvement of a functional organ (e.g., the kidney) after implantation or administration, development of a functional kidney after implantation or administration, development of improved kidney function or capacity after implantation or administration, and expression of certain markers by the native kidney following implantation or administration.

“Regenerated organ” refers to a native organ after implantation or administration of a cell population, admixture, or construct as described herein. The regenerated organ is characterized by various indicators including, without limitation, development of function or capacity in the native organ, improvement of function or capacity in the native organ, the amelioration of certain markers and physiological indices associated with disease and the expression of certain markers in the native organ. Those of ordinary skill in the art will appreciate that other indicators may be suitable for characterizing a regenerated organ.

“Regenerated kidney” refers to a native kidney after implantation or administration of a cell population, admixture, or construct as described herein. The regenerated kidney is characterized by various indicators including, without limitation, development of function or capacity in the native kidney, improvement of function or capacity in the native kidney, the amelioration of certain markers and physiological indices associated with renal disease and the expression of certain markers in the native kidney. Those of ordinary skill in the art will appreciate that other indicators may be suitable for characterizing a regenerated kidney.

### 2. Cell Populations

In certain embodiments, the formulations of the present disclosure may contain isolated, heterogeneous populations of kidney cells, and/or admixtures thereof, enriched for specific bioactive components or cell types and/or depleted of specific inactive or undesired components or cell types for use in the treatment of kidney disease, i.e., providing stabilization and/or improvement and/or regeneration of kidney function, for example as previously described in Presnell et al. U.S. Pat. No. 8,318,484 and Ilagan et al. PCT/US2011/036347, the entire contents of which are incorporated herein by reference. The formulations may contain isolated renal cell fractions that lack cellular components as compared to a healthy individual yet retain therapeutic properties, i.e., provide stabilization and/or improvement and/or regeneration of kidney function. The cell populations, cell fractions, and/or admixtures of cells described herein may be derived from healthy individuals, individuals with a kidney disease, or subjects as described herein.

The present disclosure provides formulations described herein that are suitable for use with various bioactive cell populations including, without limitation, isolated cell population(s), cell fraction(s), admixture(s), enriched cell population(s), cellular aggregate(s), organoids, tubules and other three dimensional tissue-like structures, and any combination thereof. In certain embodiments, the bioactive cell populations are bioactive renal cells. In certain embodiments, the bioactive cell populations are bioactive renal cells supplemented with endothelial cells. In certain embodiments, the bioactive cell populations are bioactive renal cells supplemented with stem or progenitor cells of mesenchymal, endothelial or epithelial lineage. In certain embodiments, the bioactive cell populations are bioactive renal cells supplemented with cells sourced from the stromal vascular fraction of adipose. In certain embodiments, only secreted products derived from bioactive cell populations are incorporated into the final construct. Such secreted products may include, without limitation, exosomes, miRNA, secreted cytokines and growth factors, extracellular vesicles, lipids and conditioned media.

Bioactive Cell Populations

In embodiments, a therapeutic composition or formulation provided herein contains an isolated, heterogeneous population of kidney cells that is enriched for specific bioactive components or cell types and/or depleted of specific inactive or undesired components or cell types. In embodiments, such compositions and formulations are used in the treatment of kidney disease, e.g., providing stabilization and/or improvement and/or regeneration of kidney function and/or structure. In embodiments, the compositions contain isolated renal cell fractions that lack cellular components as compared to a healthy individual yet retain therapeutic properties, e.g., provide stabilization and/or improvement and/or regeneration of kidney function. In embodiments, the cell populations described herein may be derived from healthy individuals, individuals with a kidney disease, or subjects as described herein.

Included herein are therapeutic compositions of selected renal cell populations that are to be administered to a target organ or tissue in a subject. In embodiments, a bioactive selected renal cell population generally refers to a cell population potentially having therapeutic properties upon administration to a subject. In embodiments, upon administration to a subject in need, a bioactive renal cell population can provide stabilization and/or improvement and/or repair and/or regeneration of kidney function in the subject. In embodiments, the therapeutic properties may include a repair or regenerative effect.

In embodiments, the renal cell population is an unfractionated, heterogeneous cell population or an enriched homogeneous cell population derived from a kidney. In embodiments, the heterogeneous cell population is isolated from a tissue biopsy or from whole organ tissue. In embodiments, the renal cell population is derived from an in vitro culture of mammalian cells, established from tissue biopsies or whole organ tissue. In embodiments, a renal cell population comprises subfractions or subpopulations of a heterogeneous population of renal cells, enriched for bioactive components (e.g., bioactive renal cells) and depleted of inactive or undesired components or cells.

In embodiments, the renal cell population expresses GGT and a cytokeratin. In embodiments, the GGT has a level of expression greater than about 10%, about 15%, about 18%, about 20%, about 25%, about 30%, about 35%, about 40%, about 45%, about 50%, about 55%, or about 60%. In embodiments, the GGT is GGT-1. In embodiments, cells of the renal cell population expresses GGT-1, a cytokeratin, VEGF, and KIM-1. In embodiments, greater than 18% of the cells in the renal cell population express GGT-1. In embodiments, greater than 80% of the cells in the renal cell population express the cytokeratin. In embodiments, the cytokeratin is selected from CK8, CK18, CK19 and combinations thereof. In embodiments, the cytokeratin is CK8, CK18, CK19, CK8/CK18, CK8/CK19, CK18/CK19 or CK8/CK18/CK19, wherein the “/” refers to a combination of the cytokeratins adjacent thereto. In embodiments, the cytokeratin has a level of expression greater than about 80%, about 85%, about 90%, or about 95%. In embodiments, greater than 80% of the cells in the renal cell population express the cytokeratin. In embodiments, the renal cell population expresses AQP2. In embodiments, less than 40% of the cells express AQP2. In embodiments, at least 3% of the cells in the renal cell population express AQP2.

In embodiments, greater than 18% of the cells within the cell population express GGT-1 and greater than 80% of the cells within the cell population express a cytokeratin. In embodiments, the cytokeratin is CK18. In embodiments, 4.5% to 81.2% of the cells in the cell population express GGT-1, 3.0% to 53.7% of the cells within the cell population express AQP2, and 81.1% to 99.7% of the cells within the cell population express CK18.

In embodiments, the renal cell population comprises cells that express one or more of any combination of the biomarkers selected from AQP1, AQP2, AQP4, Calbindin, Calponin, CD117, CD133, CD146, CD24, CD31 (PECAM-1), CD54 (ICAM-1), CD73, CK18, CK19, CK7, CK8, CK8, CK18, CK19, combinations of CK8, CK18 and CK19, Connexin 43, Cubilin, CXCR4 (Fusin), DBA, E-cadherin (CD324), EPO (erythropoeitin) GGT1, GLEPP1 (glomerular epithelial protein 1), Haptoglobulin, Itgbl (Integrin 01), KIM-1 (kidney injury molecule-1), TIM-1 (T-cell immunoglobulin and mucin-containing molecule), MAP-2 (microtubule-associated protein 2), Megalin, N-cadherin, Nephrin, NKCC (Na—K—Cl-cotransporters), OAT-1 (organic anion transporter 1), Osteopontin, Pan-cadherin, PCLP1 (podocalyxin-like 1 molecule), Podocin, SMA (smooth muscle alpha-actin), Synaptopodin, THP (tamm-horsfall protein), Vinientin, and αGST-1 (alpha glutathione S-transferase).

In embodiments, the renal cell population is enriched for epithelial cells compared to a starting population, such as a population of cells in a kidney tissue biopsy or a primary culture thereof (e.g., the renal cell population comprises at least about 5%, 10%, 15%, 20%, or 25% more epithelial cells than the starting population). In embodiments, the renal cell population is enriched for tubular cells compared to a starting population, such as a population of cells in a kidney tissue biopsy or a primary culture thereof (e.g., the renal cell population comprises at least about 5%, 10%, 15%, 20%, or 25% more tubular cells than the starting population). In embodiments, the tubular cells comprise proximal tubular cells. In embodiments, the renal cell population has a lesser proportion of distal tubular cells, collecting duct cells, endocrine cells, vascular cells, or progenitor-like cells compared to the starting population. In embodiments, the renal cell population has a lesser proportion of distal tubular cells compared to the starting population. In embodiments, the renal cell population has a lesser proportion of collecting duct cells compared to the starting population. In embodiments, the renal cell population has a lesser proportion of endocrine cells compared to the starting population. In embodiments, the renal cell population has a lesser proportion of vascular cells compared to the starting population. In embodiments, the renal cell population has a lesser proportion of progenitor-like cells compared to the starting population. In embodiments, the renal cell population has a greater proportion of tubular cells and lesser proportions of EPO producing cells, glomerular cells, and vascular cells when compared to the non-enriched population (e.g., a starting kidney cell population). In embodiments, the renal cell population has a greater proportion of tubular cells and lesser proportions of EPO producing cells and vascular cells when compared to the non-enriched population. In embodiments, the renal cell population has a greater proportion of tubular cells and lesser proportions of glomerular cells and vascular cells when compared to the non-enriched population.

In embodiments, cells of the renal cell population, express hyaluronic acid (HA). In embodiments, the size range of HA is from about 5 kDa to about 20000 kDa. In embodiments, the HA has a molecular weight of 5 kDa, 60 kDa, 800 kDa, and/or 3000 kDa. In embodiments, the renal cell population synthesizes and/or stimulate synthesis of high molecular weight HA through expression of Hyaluronic Acid Synthase-2 (HAS-2), especially after intra-renal implantation. In embodiments, cells of the renal cell population express higher molecular weight species of HA in vitro and/or in vivo, through the actions of HAS-2. In embodiments, cells of the renal cell population express higher molecular weight species of HA both in vitro and in vivo, through the actions of HAS-2. In embodiments, a higher molecular weight species of HA is HA having a molecular weight of at least 100 kDa. In embodiments, the higher molecular weight species of HA is HA having a molecular weight from about 800 kDa to about 3500 kDa. In embodiments, the higher molecular weight species of HA is HA having a molecular weight from about 800 kDa to about 3000 kDa. In embodiments, the higher molecular weight species of HA is HA having a molecular weight of at least 800 kDa. In embodiments, the higher molecular weight species of HA is HA having a molecular weight of at least 3,000 kDa. In embodiments, the higher molecular weight species of HA is HA having a molecular weight of about 800 kDa. In embodiments, the higher molecular weight species of HA is HA having a molecular weight of about 3000 kDa. In embodiments, HAS-2 synthesizes HA with a molecular weight of 2×10⁵ to 2×10⁶ Da. In embodiments, smaller species of HA are formed through the action of degradative hyaluronidases. In embodiments, the higher molecular weight species of HA is HA having a molecular weight from about 200 kDa to about 2000 kDa. In embodiments, the higher molecular weight species of HA is HA having a molecular weight of about 200 kDa. In embodiments, the higher molecular weight species of HA is HA having a molecular weight of about 2000 kDa. In embodiments, the higher molecular weight species of HA is HA having a molecular weight of at least 200 kDa. In embodiments, the higher molecular weight species of HA is HA having a molecular weight of at least 2000 kDa. In embodiments, the higher molecular weight species of HA is HA having a molecular weight of at least 5000 kDa. In embodiments, the higher molecular weight species of HA is HA having a molecular weight of at least 10000 kDa. In embodiments, the higher molecular weight species of HA is HA having a molecular weight of at least 15000 kDa. In embodiments, the higher molecular weight species of HA is HA having a molecular weight of about 20000 kDa.

In embodiments, the population comprises cells that are capable of receptor-mediated albumin transport.

In embodiments, cells of the renal cell population are hypoxia resistant.

In embodiments, the renal cell population comprises one or more cell types that express one or more of any combination of: megalin, cubilin, N-cadherin, E-cadherin, Aquaporin-1, and Aquaporin-2.

In embodiments, the renal cell population comprises one or more cell types that express one or more of any combination of: megalin, cubilin, hyaluronic acid synthase 2 (HAS2), Vitamin D3 25-Hydroxylase (CYP2D25), N-cadherin (Ncad), E-cadherin (Ecad), Aquaporin-1 (Aqp1), Aquaporin-2 (Aqp2), RAB17, member RAS oncogene family (Rab17), GATA binding protein 3 (Gata3), FXYD domain-containing ion transport regulator 4 (Fxyd4), solute carrier family 9 (sodium/hydrogen exchanger), member 4 (Slc9a4), aldehyde dehydrogenase 3 family, member B1 (Aldh3b1), aldehyde dehydrogenase 1 family, member A3 (Aldh1a3), and Calpain-8 (Capn8).

In embodiments, the renal cell population comprises one or more cell types that express one or more of any combination of: megalin, cubilin, hyaluronic acid synthase 2 (HAS2), Vitamin D3 25-Hydroxylase (CYP2D25), N-cadherin (Ncad), E-cadherin (Ecad), Aquaporin-1 (Aqp1), Aquaporin-2 (Aqp2), RAB17, member RAS oncogene family (Rab17), GATA binding protein 3 (Gata3), FXYD domain-containing ion transport regulator 4 (Fxyd4), solute carrier family 9 (sodium/hydrogen exchanger), member 4 (Slc9a4), aldehyde dehydrogenase 3 family, member 81 (Aldh3b1), aldehyde dehydrogenase 1 family, member A3 (Aldh1a3), and Calpain-8 (Capn8), and Aquaporin-4 (Aqp4).

In embodiments, the renal cell population comprises one or more cell types that express one or more of any combination of: aquaporin 7 (Aqp7), FXYD domain-containing ion transport regulator 2 (Fxyd2), solute carrier family 17 (sodium phosphate), member 3 (Slc17a3), solute carrier family 3, member 1 (Slc3a1), claudin 2 (Cldn2), napsin A aspartic peptidase (Napsa), solute carrier family 2 (facilitated glucose transporter), member 2 (Slc2a2), alanyl (membrane) aminopeptidase (Anpep), transmembrane protein 27 (Tmem27), acyl-CoA synthetase medium-chain family member 2 (Acsm2), glutathione peroxidase 3 (Gpx3), fructose-1,6-biphosphatase 1 (Fbp1), alanine-glyoxylate aminotransferase 2 (Agxt2), platelet endothelial cell adhesion molecule (Pecam), and podocin (Podn).

In embodiments, the renal cell population comprises one or more cell types that express one or more of any combination of: PECAM, VEGF, KDR, HIF1a, CD31, CD146, Podocin (Podn), and Nephrin (Neph), chemokine (C-X-C motif) receptor 4 (Cxcr4), endothelin receptor type B (Ednrb), collagen, type V, alpha 2 (Col5a2), Cadherin 5 (Cdh5), plasminogen activator, tissue (Plat), angiopoietin 2 (Angpt2), kinase insert domain protein receptor (Kdr), secreted protein, acidic, cysteine-rich (osteonectin) (Sparc), serglycin (Srgn), TIMP metallopeptidase inhibitor 3 (Timp3), Wilms tumor 1 (Wt1), wingless-type MMTV integration site family, member 4 (Wnt4), regulator of G-protein signaling 4 (Rgs4), Erythropoietin (EPO).

In embodiments, the renal cell population comprises one or more cell types that express one or more of any combination of: PECAM, vEGF, KDR, HIF1a, podocin, nephrin, EPO, CK7, CK8/18/19.

In embodiments, the renal cell population comprises one or more cell types that express one or more of any combination of: PECAM, vEGF, KDR, HIF1a, CD31, CD146.

In embodiments, the renal cell population comprises one or more cell types that express one or more of any combination of: Podocin (Podn), and Nephrin (Neph).

In embodiments, the renal cell population comprises one or more cell types that express one or more of any combination of: PECAM, vEGF, KDR, HIF1a, and EPO.

In embodiments, the presence (e.g., expression) and/or level/amount of various biomarkers in a sample or cell population can be analyzed by a number of methodologies, many of which are known in the art and understood by the skilled artisan, including, but not limited to, immunohistochemical (“IHC”), Western blot analysis, immunoprecipitation, molecular binding assays, ELISA, ELIFA, fluorescence activated cell sorting (“FACS”), MassARRAY, proteomics, biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Non-limiting examples of protocols for evaluating the status of genes and gene products include Northern Blotting, Southern Blotting, Immunoblotting, and PCR Analysis. In embodiments, multiplexed immunoassays such as those available from Rules Based Medicine or Meso Scale Discovery may also be used. In embodiments, the presence (e.g., expression) and/or level/amount of various biomarkers in a sample or cell population can be analyzed by a number of methodologies, many of which are known in the art and understood by the skilled artisan, including, but not limited to, “-omics” platforms such as genome-wide transcriptomics, proteomics, secretomics, lipidomics, phospatomics, exosomics etc., wherein high-throughput methodologies are coupled with computational biology and bioinformatics techniques to elucidate a complete biological signature of genes, miRNA, proteins, secreted proteins, lipids, etc. that are expressed and not expressed by the cell population under consideration.

In embodiments, a method of detecting the presence of two or more biomarkers in a renal cell population comprises contacting the sample with an antibody directed to a biomarker under conditions permissive for binding of the antibody to its cognate ligand (i.e., biomarker), and detecting the presence of the bound antibody, e.g., by detecting whether a complex is formed between the antibody and the biomarker. In embodiments, the detection of the presence of one or more biomarkers is by immunohistochemistry. The term “detecting” as used herein encompasses quantitative and/or qualitative detection.

In embodiments, a renal cell population are identified with one or more reagents that allow detection of a biomarker disclosed herein, such as AQP1, AQP2, AQP4, Calbindin, Calponin, CD117, CD133, CD146, CD24, CD31 (PECAM-1), CD54 (ICAM-1), CD73, CK18, CK19, CK7, CK8, CK8/18, CK8/18/19, Connexin 43, Cubilin, CXCR4 (Fusin), DBA, E-cadherin (CD324), EPO (erythropoeitin), GGT1, GLEPP1 (glomerular epithelial protein 1), Haptoglobulin, Itgbl (Integrin p), KIM-1 (kidney injury molecule-1), TIM-1 (T-cell immunoglobulin and mucirs-containing molecule), MAP-2 (microtubule-associated protein 2), Megalin, N-cadherin, Nephrin, NKCC (Na—K—Cl-cotransporters), OAT-1 (organic anion transporter 1), Osteopontin, Pan-cadherin, PCLP1 (podocalyxin-like 1 molecule), Podocin, SMA (smooth muscle alpha-actin), Synaptopodin, THP (tamm-horsfall protein), Vimentin, and αGST-1 (alpha glutathione 5-transferase). In embodiments, a biomarker is detected by a monoclonal or polyclonal antibody.

In embodiments, the source of cells is the same as the intended target organ or tissue. In embodiments, BRCs or SRCs may be sourced from the kidney to be used in a formulation to be administered to the kidney. In embodiments, the cell population is derived from a kidney biopsy. In embodiments, a cell populations is derived from whole kidney tissue. In embodiments, a cell population is derived from in vitro cultures of mammalian kidney cells, established from kidney biopsies or whole kidney tissue.

In embodiments, the BRCs or SRCs comprise heterogeneous mixtures or fractions of bioactive renal cells. In embodiments, the BRCs or SRCs may be derived from or are themselves renal cell fractions from healthy individuals. In embodiments, included herein is a renal cell population or fraction obtained from an unhealthy individual that may lack certain cell types when compared to the renal cell population of a healthy individual (e.g., in a kidney or biopsy thereof). In embodiments, provided herein is a therapeutically-active cell population lacking cell types compared to a healthy individual. In embodiments, a cell population is isolated and expanded from an autologous cell population.

In embodiments, SRCs are obtained from isolation and expansion of renal cells from a patient's renal cortical tissue via a kidney biopsy. In embodiments, renal cells are isolated from the kidney tissue by enzymatic digestion, expanded using standard cell culture techniques, and selected by centrifugation across a density boundary, barrier, or interface from the expanded renal cells. In embodiments, renal cells are isolated from the kidney tissue by enzymatic digestion, expanded using standard cell culture techniques, and selected by continuous or discontinuous single or multistep density gradient centrifugation from the expanded renal cells. In embodiments, SRCs are composed primarily of renal epithelial cells which are known for their regenerative potential. In embodiments, other parenchymal (vascular) and stromal cells may be present in the autologous SRC population.

In embodiments, bioactive renal cells are obtained from renal cells isolated from kidney tissue by enzymatic digestion and expanded using standard cell culture techniques. In embodiments, the cell culture medium is designed to expand bioactive renal cells with regenerative capacity. In embodiments, the cell culture medium does not contain any recombinant or purified differentiation factors. In embodiments, the expanded heterogeneous mixtures of renal cells are cultured in hypoxic conditions to further enrich the composition of cells with regenerative capacity. Without wishing to be bound by theory, this may be due to one or more of the following phenomena: 1) selective survival, death, or proliferation of specific cellular components during the hypoxic culture period; 2) alterations in cell granularity and/or size in response to the hypoxic culture, thereby effecting alterations in buoyant density and subsequent localization during density gradient separation or during centrifugation across a density boundary, barrier, or interface; and 3) alterations in cell gene/protein expression in response to the hypoxic culture period, thereby resulting in differential characteristics of the cells within the isolated and expanded population.

In embodiments, the bioactive renal cell population is obtained from isolation and expansion of renal cells from kidney tissue (such as tissue obtained from a biopsy) under culturing conditions that enrich for cells capable of kidney regeneration.

In embodiments, renal cells from kidney tissue (such as tissue obtained from a biopsy) are passaged 1, 2, 3, 4, 5, or more times to produce expanded bioactive renal cells (such as a cell population enriched for cells capable of kidney regeneration). In embodiments, renal cells from kidney tissue (such as tissue obtained from a biopsy) are passaged 1 time to produce expanded bioactive renal cells. In embodiments, renal cells from kidney tissue (such as tissue obtained from a biopsy) are passaged 2 times to produce expanded bioactive renal cells. In embodiments, renal cells from kidney tissue (such as tissue obtained from a biopsy) are passaged 3 times to produce expanded bioactive renal cells. In embodiments, renal cells from kidney tissue (such as tissue obtained from a biopsy) are passaged 4 times to produce expanded bioactive renal cells. In embodiments, renal cells from kidney tissue (such as tissue obtained from a biopsy) are passaged 5 times to produce expanded bioactive renal cells. In embodiments, passaging the cells depletes the cell population of non-bioactive renal cells. In embodiments, passaging the cells depletes the cell population of at least one cell type. In embodiments, passaging the cells depletes the cell population of cells having a density greater than 1.095 g/ml. In embodiments, passaging the cells depletes the cell population of small cells of low granularity. In embodiments, passaging the cells depletes the cell population of cells that are smaller than erythrocytes. In embodiments, passaging the cells depletes the cell population of cells with a diameter of less than 6 μm. In embodiments, passaging cells depletes cell population of cells with a diameter less than 2 μm. In embodiments, passaging the cells depletes the cell population of cells with lower granularity than erythrocytes. In embodiments, the viability of the cell population increases after 1 or more passages. In embodiments, descriptions of small cells and low granularity are used when analyzing cells by fluorescence activated cell sorting (FACs), e.g., using the X-Y axis of a scatter-plot of where the cells show up.

In embodiments, the expanded bioactive renal cells are grown under hypoxic conditions for at least about 6, 9, 10, 12, or 24 hours but less than 48 hours, or from 6 to 9 hours, or from 6 to 48 hours, or from about 12 to about 15 hours, or about 8 hours, or about 12 hours, or about 24 hours, or about 36 hours, or about 48 hours. In embodiments, cells grown under hypoxic conditions are selected based on density. In embodiments, the bioactive renal cell population is a selected renal cell (SRC) population obtained after continuous or discontinuous (single step or multistep) density gradient separation of the expanded renal cells (e.g., after passaging and/or culture under hypoxic conditions). In embodiments, the bioactive renal cell population is a selected renal cell (SRC) population obtained after separation of the expanded renal cells by centrifugation across a density boundary, barrier, or interface (e.g., after passaging and/or culture under hypoxic condutions). In embodiments, a hypoxic culture condition is a culture condition in which cells are subjected to a reduction in available oxygen levels in the culture system relative to standard culture conditions in which cells are cultured at atmospheric oxygen levels (about 21%). In embodiments, cells cultured under hypoxic culture conditions are cultured at an oxygen level of about 5% to about 15%, or about 5% to about 10%, or about 2% to about 5%, or about 2% to about 7%, or about 2% or about 3%, or about 4%, or about 5%. In embodiments, the SRCs exhibit a buoyant density greater than approximately 1.0419 g/mL. In embodiments, the SRCs exhibit a buoyant density greater than approximately 1.04 g/mL. In embodiments, the SRCs exhibit a buoyant density greater than approximately 1.045 g/mL. In embodiments, the BRCs or SRCs contain a greater percentage of one or more cell populations and lacks or is deficient in one or more other cell populations, as compared to a starting kidney cell population.

In embodiments, expanded bioactive renal cells may be subjected to density gradient separation to obtain SRCs. In embodiments, continuous or discontinuous single step or multistep density gradient centrifugation is used to separate harvested renal cell populations based on cell buoyant density. In embodiments, expanded bioactive renal cells may be separated by centrifugation across a density boundary, barrier or interface to obtain SRCs. In embodiments, centrifugation across a density boundary or interface is used to separate harvested renal cell populations based on cell buoyant density. In embodiments, the SRCs are generated by using, in part, OPTIPREP (Axis-Shield) medium, comprising a solution of 60% (w/v) of the nonionic iodinated compound iodixanol in water. One of skill in the art, however, will recognize that other media, density gradients (continuous or discontinuous), density boundaries, barriers, interfaces or other means, e.g., immunological separation using cell surface markers known in the art, comprising necessary features for isolating cell populations described herein may be used to obtain bioactive renal cells. In embodiments, a cellular fraction exhibiting buoyant density greater than approximately 1.04 g/mL is collected after centrifugation as a distinct pellet. In embodiments, cells maintaining a buoyant density of less than 1.04 g/mL are excluded and discarded. In embodiments, a cellular fraction exhibiting buoyant density greater than approximately 1.0419 g/mL is collected after centrifugation as a distinct pellet. In embodiments, cells maintaining a buoyant density of less than 1.0419 g/mL are excluded and discarded. In embodiments, a cellular fraction exhibiting buoyant density greater than approximately 1.045 g/mL is collected after centrifugation as a distinct pellet. In embodiments, cells maintaining a buoyant density of less than 1.045 g/mL are excluded and discarded.

In embodiments, cell buoyant density is used to obtain an SRC population and/or to determine whether a renal cell population is a bioactive renal cell population. In embodiments, cell buoyant density is used to isolate bioactive renal cells. In embodiments, cell buoyant density is determined by centrifugation across a single-step OptiPrep (7% iodixanol; 60% (w/v) in OptiMEM) density interface (single step discontinuous density gradient). Optiprep is a 60% w/v solution of iodixanol in water. When used in an exemplary density interface or single step discontinuous density gradient, the Optiprep is diluted with OptiMEM (a cell culturing basal medium) to form a final solution of 7% iodixanol (in water and OptiMEM). The formulation of OptiMEM is a modification of Eagle's Minimal Essential Medium, buffered with HEPES and sodium bicarbonate, and supplemented with hypoxanthine, thymidine, sodium pyruvate, L-glutamine or GLUTAMAX, trace elements and growth factors. The protein level is minimal (15 μg/mL), with insulin and transferrin being the only protein supplements. Phenol red is included at a reduced concentration as a pH indicator. In embodiments, OptiMEM may be supplemented with 2-mercaptoethanol prior to use.

In embodiments, the OptiPrep solution is prepared and refractive index indicative of desired density is measured (R.I. 1.3456+/−0.0004) prior to use. In embodiments, renal cells are layered on top of the solution. In embodiments, the density interface or single step discontinuous density gradient is centrifuged at 800 g for 20 min at room temperature (without brake) in either a centrifuge tube (e.g., a 50 ml conical tube) or a cell processor (e.g. COBE 2991). In embodiments, the cellular fraction exhibiting buoyant density greater than approximately 1.04 g/mL is collected after centrifugation as a distinct pellet. In embodiments, cells maintaining a buoyant density of less than 1.04 g/mL are excluded and discarded. In embodiments, the cellular fraction exhibiting buoyant density greater than approximately 1.0419 g/mL is collected after centrifugation as a distinct pellet. In embodiments, cells maintaining a buoyant density of less than 1.0419 g/mL are excluded and discarded. In embodiments, the cellular fraction exhibiting buoyant density greater than approximately 1.045 g/mL is collected after centrifugation as a distinct pellet. In embodiments, cells maintaining a buoyant density of less than 1.045 g/mL are excluded and discarded. In embodiments, prior to the assessment of cell density or selection based on density, cells are cultured until they are at least 50% confluent and incubated overnight (e.g., at least about 8 or 12 hours) in a hypoxic incubator set for 2% oxygen in a 5% CO₂ environment at 37° C.

In embodiments, cells obtained from a kidney sample are expanded and then processed (e.g. by hypoxia and centrifugation separation) to provide a SRC population. In embodiments, an SRC population is produced using reagents and procedures described herein. In embodiments, a sample of cells from an SRC population is tested for viability before cells of the population are administration to a subject. In embodiments, a sample of cells from an SRC population is tested for the expression of one or more of the markers disclosed herein before cells of the population administration to a subject.

Non-limiting examples of compositions and methods for preparing SRCs are disclosed in U.S. Patent Application Publication No. 2017/0281684 A1, the entire content of which is incorporated herein by reference.

In embodiments, the BRCs or SRCs are derived from a native autologous or allogeneic kidney sample. In embodiments, the BRCs or SRCs are derived from a non-autologous kidney sample. In embodiments, the sample may be obtained by kidney biopsy.

In embodiments, renal cell isolation and expansion provides a mixture of renal cell types including renal epithelial cells and stromal cells. In embodiments, SRC are obtained by continuous or discontinuous density gradient separation of the expanded renal cells. In embodiments, the primary cell type in the density gradient separated SRC population is of tubular epithelial phenotype. In embodiments, SRC are obtained by separation of the expanded renal cells by centrifugation across a density boundary, barrier, or interface. In embodiments, the primary cell type in the SRC population separated across a density boundary/barrier/interface is of tubular epithelial phenotype. In embodiments, the characteristics of SRC obtained from expanded renal cells are evaluated using a multi-pronged approach. In embodiments, cell morphology, growth kinetics and cell viability are monitored during the renal cell expansion process. In embodiments, SRC buoyant density and viability is characterized by centrifugation on or through a density gradient medium and Trypan Blue exclusion. In embodiments, SRC phenotype is characterized by flow cytometry and SRC function is demonstrated by expression of VEGF and KIM-1. In embodiments, cell function of SRC, pre-formulation, can also be evaluated by measuring the activity of two specific enzymes; GGT (γ-glutamyl transpeptidase) and LAP (leucine aminopeptidase), found in kidney proximal tubules.

In embodiments, cellular features that contribute to separation of cellular subpopulations via a density medium (size and granularity) can be exploited to separate cellular subpopulations via flow cytometry (forward scatter=a reflection of size via flow cytometry, and side scatter=a reflection of granularity). In embodiments, a density gradient or separation medium should have low toxicity towards the specific cells of interest. In embodiments, while the density medium should have low toxicity toward the specific cells of interest, the instant disclosure contemplates the use of mediums which play a role in the selection process of the cells of interest. In embodiments, and without wishing to be bound by theory, it appears that the cell populations disclosed herein recovered by the medium comprising iodixanol are iodixanol-resistant, as there is an appreciable loss of cells between the loading and recovery steps, suggesting that exposure to iodixanol under the conditions of the density gradient or density boundary, density, barrier, or density interface leads to elimination of certain cells. In embodiments, cells appearing after an iodixanol density gradient or density interface separation are resistant to any untoward effects of iodixanol and/or density gradient or interface exposure. In embodiments, a contrast medium comprising a mild to moderate nephrotoxin is used in the isolation and/or selection of a cell population, e.g. a SRC population. In embodiments, SRCs are iodixanol-resistant. In embodiments, the density medium should not bind to proteins in human plasma or adversely affect key functions of the cells of interest.

In embodiments, a cell population has been enriched and/or depleted of one or more kidney cell types using fluorescent activated cell sorting (FACS). In embodiments, kidney cell types may be enriched and/or depleted using BD FACSAria™ or equivalent. In embodiments, kidney cell types may be enriched and/or depleted using FACSAria III™ or equivalent.

In embodiments, a cell population has been enriched and/or depleted of one or more kidney cell types using magnetic cell sorting. In embodiments, one or more kidney cell types may be enriched and/or depleted using the Miltenyi autoMACS® system or equivalent.

In embodiments, a renal cell population has been subject to three-dimensional culturing. In embodiments, the methods of culturing the cell populations are via continuous perfusion. In embodiments, the cell populations cultured via three-dimensional culturing and continuous perfusion demonstrate greater cellularity and interconnectivity when compared to cell populations cultured statically. In embodiments, the cell populations cultured via three dimensional culturing and continuous perfusion demonstrate greater expression of EPO, as well as enhanced expression of renal tubule-associate genes such as E-cadherin when compared to static cultures of such cell populations. In embodiments, a cell population cultured via continuous perfusion demonstrates a greater level of glucose and glutamine consumption when compared to a cell population cultured statically.

In embodiments, low or hypoxic oxygen conditions may be used in the methods to prepare a cell population provided for herein. In embodiments, a method of preparing a cell population may be used without the step of low oxygen conditioning. In embodiments, normoxic conditions may be used.

In embodiments, a renal cell population has been isolated and/or cultured from kidney tissue. Non-limiting examples of methods are disclosed herein for separating and isolating the renal cellular components, e.g., enriched cell populations that will be used in the formulations for therapeutic use, including the treatment of kidney disease, anemia, EPO deficiency, tubular transport deficiency, and glomerular filtration deficiency. In embodiments, a cell population is isolated from freshly digested, i.e., mechanically or enzymatically digested, kidney tissue or from a heterogeneous in vitro culture of mammalian kidney cells.

In embodiments, the renal cell population comprises EPO-producing kidney cells. In embodiments, a subject has anemia and/or EPO deficiency. In embodiments, EPO-producing kidney cell populations that are characterized by EPO expression and bioresponsiveness to oxygen, such that a reduction in the oxygen tension of the culture system results in an induction in the expression of EPO. In embodiments, the EPO-producing cell populations are enriched for EPO-producing cells. In embodiments, the EPO expression is induced when the cell population is cultured under conditions where the cells are subjected to a reduction in available oxygen levels in the culture system as compared to a cell population cultured at normal atmospheric (about 21%) levels of available oxygen. In embodiments, EPO-producing cells cultured in lower oxygen conditions express greater levels of EPO relative to EPO-producing cells cultured at normal oxygen conditions. In general, the culturing of cells at reduced levels of available oxygen (also referred to as hypoxic culture conditions) means that the level of reduced oxygen is reduced relative to the culturing of cells at normal atmospheric levels of available oxygen (also referred to as normal or normoxic culture conditions). In embodiments, hypoxic cell culture conditions include culturing cells at about less than 1% oxygen, about less than 2% oxygen, about less than 3% oxygen, about less than 4% oxygen, or about less than 5% oxygen. In embodiments, normal or normoxic culture conditions include culturing cells at about 10% oxygen, about 12% oxygen, about 13% oxygen, about 14% oxygen, about 15% oxygen, about 16% oxygen, about 17% oxygen, about 18% oxygen, about 19% oxygen, about 20% oxygen, or about 21% oxygen.

In embodiments, induction or increased expression of EPO is obtained and can be observed by culturing cells at about less than 5% available oxygen and comparing EPO expression levels to cells cultured at atmospheric (about 21%) oxygen. In embodiments, the induction of EPO is obtained in a culture of cells capable of expressing EPO by a method that includes a first culture phase in which the culture of cells is cultivated at atmospheric oxygen (about 21%) for some period of time and a second culture phase in which the available oxygen levels are reduced and the same cells are cultured at about less than 5% available oxygen. In embodiments, the EPO expression that is responsive to hypoxic conditions is regulated by HIF1α. In embodiments, other oxygen manipulation culture conditions known in the art may be used for the cells described herein.

In embodiments, the formulation contains enriched populations of EPO-producing mammalian cells characterized by bio-responsiveness (e.g., EPO expression) to perfusion conditions. In embodiments, the perfusion conditions include transient, intermittent, or continuous fluid flow (perfusion). In embodiments, the EPO expression is mechanically-induced when the media in which the cells are cultured is intermittently or continuously circulated or agitated in such a manner that dynamic forces are transferred to the cells via the flow. In embodiments, the cells subjected to the transient, intermittent, or continuous fluid flow are cultured in such a manner that they are present as three-dimensional structures in or on a material that provides framework and/or space for such three-dimensional structures to form. In embodiments, the cells are cultured on porous beads and subjected to intermittent or continuous fluid flow by means of a rocking platform, orbiting platform, or spinner flask. In embodiments, the cells are cultured on three-dimensional scaffolding and placed into a device whereby the scaffold is stationary and fluid flows directionally through or across the scaffolding. Those of ordinary skill in the art will appreciate that other perfusion culture conditions known in the art may be used for the cells described herein.

In embodiments, a cell population is derived from a kidney biopsy. In embodiments, a cell population is derived from whole kidney tissue. In embodiments, a cell population is derived from an in vitro culture of mammalian kidney cells, established from kidney biopsies or whole kidney tissue. In embodiments, the renal cell population is a SRC population. In embodiments, a cell population is an unfractionated cell populations, also referred to herein as a non-enriched cell population.

Compositions containing a variety of active agents (e.g., other than renal cells) are included herein. Non-limiting examples of suitable active agents include, without limitation, cellular aggregates, acellular biomaterials, secreted products from bioactive cells, large and small molecule therapeutics, as well as combinations thereof. For example, one type of bioactive cells may be combined with biomaterial-based microcarriers with or without therapeutic molecules or another type of bioactive cells. In embodiments, unattached cells may be combined with acellular particles.

In embodiments, cells of the renal cell population are within spheroids. In embodiments, the renal cell population is in the form of spheroids. In embodiments, spheroids comprising bioactive renal cells are administered to a subject. In embodiments, the spheroids comprise at least one non-renal cell type or population of cells. In embodiments, the a spheroids are produced in a method comprising (i) combining a bioactive renal cell population and a non-renal cell population, and (ii) culturing the bioactive renal cell population and the non-renal cell population in a 3-dimensional culture system comprising a spinner flask until the spheroids form.

In embodiments, the non-renal cell population comprises an endothelial cell population or an endothelial progenitor cell population. In embodiments, the bioactive cell population is an endothelial cell population. In embodiments, the endothelial cell population is a cell line. In embodiments, the endothelial cell population comprises human umbilical vein endothelial cells (HUVECs). In embodiments, the non-renal cell population is a mesenchymal stem cell population. In embodiments, the non-renal cell population is a stem cell population of hematopoietic, mammary, intestinal, placental, lung, bone marrow, blood, umbilical cord, endothelial, dental pulp, adipose, neural, olfactory, neural crest, or testicular origin. In embodiments, the non-renal cell population is an adipose-derived progenitor cell population. In embodiments, the cell populations are xenogeneic, syngeneic, allogeneic, autologous or combinations thereof. In embodiments, the bioactive renal cell population and non-renal cell population are cultured at a ratio of from 0.1:9.9 to 9.9:0.1. In embodiments, the bioactive renal cell population and non-renal cell population are cultured at a ratio of about 1:1. In embodiments, the renal cell population and bioactive cell population are suspended in growth medium.

The expanded bioactive renal cells may be further subjected to continuous or discontinuous density medium separation to obtain the SRC. Specifically, continuous or discontinuous single step or multistep density gradient centrifugation is used to separate harvested renal cell populations based on cell buoyant density. In certain embodiments, the expanded bioactive renal cells may be further subjected to separation by centrifugation across a density boundary, barrier, or interface to obtain the SRC. Specifically, centrifugation across a density boundary, barrier, or interface is used to separate harvested renal cell populations based on cell buoyant density. In certain embodiments, the SRC are generated by using, in part, the OPTIPREP (Axis-Shield) medium, comprising a 60% solution of the nonionic iodinated compound iodixanol in water. One of skill in the art, however, will recognize that any density gradient medium without limitation of specific medium or other means, e.g., immunological separation using cell surface markers known in the art, comprising necessary features for isolating the cell populations of the instant disclosure may be used in accordance with the disclosure. For example, Percoll or sucrose may be used to form a density gradient or density boundary. In certain embodiments, the cellular fraction exhibiting buoyant density greater than approximately 1.04 g/mL is collected after centrifugation as a distinct pellet. In certain embodiments, cells maintaining a buoyant density of less than 1.04 g/mL are excluded and discarded. In certain embodiments, the cellular fraction exhibiting buoyant density greater than approximately 1.0419 g/mL is collected after centrifugation as a distinct pellet. In certain embodiments, cells maintaining a buoyant density of less than 1.0419 g/mL are excluded and discarded. In certain embodiments, the cellular fraction exhibiting buoyant density greater than approximately 1.045 g/mL is collected after centrifugation as a distinct pellet. In certain embodiments, cells maintaining a buoyant density of less than 1.045 g/mL are excluded and discarded.

The therapeutic compositions, and formulations thereof, of the present disclosure may contain isolated, heterogeneous populations of kidney cells, and/or admixtures thereof, enriched for specific bioactive components or cell types and/or depleted of specific inactive or undesired components or cell types for use in the treatment of kidney disease, i.e., providing stabilization and/or improvement and/or regeneration of kidney function and/or structure, for example a previously described in Presnell et al. U.S. Pat. No. 8,318,484 and Ilagan et al. PCT/US2011/036347, the entire contents of which are incorporated herein by reference. The compositions may contain isolated renal cell fractions that lack cellular components as compared to a healthy individual yet retain therapeutic properties, i.e., provide stabilization and/or improvement and/or regeneration of kidney function. The cell populations, cell fractions, and/or admixtures of cells described herein may be derived from healthy individuals, individuals with a kidney disease, or subjects as described herein.

The present disclosure contemplates therapeutic compositions of selected renal cell populations that are to be administered to target organs or tissue in a subject in need. A bioactive selected renal cell population generally refers to a cell population potentially having therapeutic properties upon administration to a subject. For example, upon administration to a subject in need, a bioactive renal cell population can provide stabilization and/or improvement and/or repair and/or regeneration of kidney function in the subject. The therapeutic properties may include a regenerative effect.

In certain embodiments, the source of cells is the same as the intended target organ or tissue. For example, BRCs and/or SRCs may be sourced from the kidney to be used in a formulation to be administered to the kidney. In certain embodiments, the cell populations are derived from a kidney biopsy. In certain embodiments, the cell populations are derived from whole kidney tissue. In one other embodiment, the cell populations are derived from in vitro cultures of mammalian kidney cells, established from kidney biopsies or whole kidney tissue. In certain embodiments, the BRCs and/or SRCs comprise heterogeneous mixtures or fractions of bioactive renal cells. The BRCs and/or SRCs may be derived from or are themselves renal cell fractions from healthy individuals. In addition, the present disclosure provides renal cell fractions obtained from an unhealthy individual that may lack certain cellular components when compared to the corresponding renal cell fractions of a healthy individual, yet still retain therapeutic properties. The present disclosure also provides therapeutically-active cell populations lacking cellular components compared to a healthy individual, which cell populations can be, in certain embodiments, isolated and expanded from autologous sources in various disease states.

In certain embodiments, the SRCs are obtained from isolation and expansion of renal cells from a patient's renal cortical tissue via a kidney biopsy. Renal cells are isolated from the kidney tissue by enzymatic digestion, expanded using standard cell culture techniques, and selected by centrifugation of the expanded renal cells across a density boundary, barrier, or interface. In this embodiment, SRC are composed primarily of renal tubular epithelial cells which are known for their regenerative potential (Bonventre J V. Dedifferentiation and proliferation of surviving epithelial cells in acute renal failure. J Am Soc Nephrol. 2003; 14(Suppl. 1):555-61; Humphreys B D, Czerniak S, DiRocco D P, et al. Repair of injured proximal tubule does not involve specialized progenitors. PNAS. 2011; 108:9226-31; Humphreys B D, Valerius M T, Kobayashi A, et al. Intrinsic epithelial cells repair the kidney after injury. Cell Stem Cell. 2008; 2:284-91). Other parenchymal (vascular) and stromal cells may be present in the autologous SRC population. In certain embodiments, renal cells are selected by centrifugation through a continuous or discontinuous single step or multistep gradient.

As described herein, the present disclosure is based, in part, on the surprising finding that certain subfractions of a heterogeneous population of renal cells, enriched for bioactive components and depleted of inactive or undesired components, provide superior therapeutic and regenerative outcomes than the starting population.

Renal cell isolation and expansion provides a mixture of renal cell types including renal tubular epithelial cells and stromal cells. As noted above, SRC are obtained by separation of the expanded renal cells by centrifugation across a density boundary, barrier, or interface. The primary cell type in the separated SRC population is of tubular epithelial phenotype. The characteristics of SRC obtained from expanded renal cells is evaluated using a multi-pronged approach. Cell morphology, growth kinetics and cell viability are monitored during the renal cell expansion process. SRC buoyant density and viability is characterized by density interface and Trypan Blue exclusion. SRC phenotype is characterized by flow cytometry and SRC function is demonstrated by expression of VEGF and KIM-1.

Those of ordinary skill in the art will appreciate that other methods of isolation and culturing known in the art may be used for the cells described herein. Those of ordinary skill in the art will also appreciate that bioactive cell populations may be derived from sources other than those specifically listed above, including, without limitation, tissues and organs other than the kidney, body fluids and adipose.

SRC Phenotype

In certain embodiments, cell phenotype is monitored by expression analysis of renal cell markers using flow cytometry. Phenotypic analysis of cells is based on the use of antigenic markers specific for the cell type being analyzed. Flow cytometric analysis provides a quantitative measure of cells in the sample population which express the antigenic marker being analyzed.

A variety of markers have been reported in the literature as being useful for phenotypic characterization of renal cells: (i) cytokeratins; (ii) transport membrane proteins (aquaporins and cubilin); (iii) cell binding molecules (adherins, lectins, and other proteins); and (iv) metabolic enzymes (glutathione and gamma-glutamyl transpeptidase (GGT)). (Table 1) Since the majority of cells found in cultures derived from whole kidney digests are epithelial and endothelial cells, the markers examined focus on the expression of proteins generally associated with these two groups.

TABLE 1
Phenotypic Markers for SRC Characterization

| Antigenic marker | Reactivity |
|---|---|
| CK8/18/19 | Epithelial cells, proximal and distal tubules |
| CK8 | Epithelial cells, proximal tubules |
| CK18 | Epithelial cells, proximal tubules |
| CK19 | Epithelial cells, collecting ducts, distal tubules |
| CK7 | Epithelial cells, collecting ducts, distal tubules |
| CXCR4 | Epithelial cells, distal and proximal tubules |
| E-cadherin | Epithelial cells, distal tubules |
| Cubilin | Epithelial cells, proximal tubules |
| Aquaporin1 | Epithelial cells, proximal tubules, descending |
| | thin limb |
| GGT1 | Fetal and adult kidney cells, proximal tubules |
| Aquaporin2 | Renal collecting duct cells, distal tubules |
| DBA | Renal collecting duct cells, distal tubules |
| CD31 | Endothelial cells of the kidney (rat) |
| CD146 | Endothelial cells of the kidney (canine, human) |

Table 2 provides selected markers, range and mean percentage values of phenotypic in the SRC population and the rationale for their selection.

TABLE 2
Marker Selected for Phenotypic Analysis of SRC

| Phenotypic | Expression | | | Expression |
|---|---|---|---|---|
| Marker | Range | Average | Rationale | Level |
| CK18 | 81.1 to 99.7% | 96.7% | Epithelial marker | High |
| | (n = 87) | | | |
| GGT1 | 4.5 to 81.2% | 50.7% | Functional Tubular | Moderate |
| | (n = 63) | | marker | |

Cell Function

SRC actively secrete proteins which can be detected through analysis of conditioned medium. Cell function is assessed by the ability of cells to metabolize PrestoBlue and to secrete VEGF (Vascular Endothelial Growth Factor) and KIM-1 (Kidney Injury Molecule-1).

Table 3 presents VEGF and KIM-1 quantities present in conditioned medium from renal cells and SRC cultures. Renal cells were cultured to near confluence. Conditioned medium from overnight exposure to the renal cell cultures was tested for VEGF and KIM-1.

TABLE 3
Production of VEGF and KIM-1 by Human Renal Cells and SRC

| | VEGF | | KIM-1 | |
|---|---|---|---|---|
| Conditioned | | ng/million | | ng/million |
| Medium | ng/mL | cells | ng/mL | cells |
| Renal Cell | 0.50 to 2.42 | 2.98 to 14.6 | 0.20 to 3.41 | 1.14 to 15.2 |
| Culture | | | | |
| (n = 15) | | | | |
| SRC | 0.80 to 3.85 | 4.83 to 23.07 | 0.32 to 2.10 | 1.93 to 12.59 |
| (n = 14) | | | | |

SRC Enzymatic Activity

Cell function of SRC, pre-formulation, can also be evaluated by measuring the activity of two specific enzymes; GGT (γ-glutamyl transpeptidase) and LAP (leucine aminopeptidase), found in kidney proximal tubules.

Although selected renal cell compositions are described herein, the present disclosure contemplates compositions containing a variety of other active agents including cells and admixtures of cells sourced from tissues and organs other than the kidney. Other suitable active agents include, without limitation, cellular aggregates and organoids, acellular biomaterials, secreted products from bioactive cells, large and small molecule therapeutics, as well as combinations thereof. For example, one type of bioactive cell may be combined with biomaterial-based microcarriers with or without therapeutic molecules or another type of bioactive cell. In certain embodiments, unattached cells may be combined with acellular particles.

Cellular Aggregates

In one other aspect, the formulations of the present disclosure contain cellular aggregates or spheroids. In certain embodiments, the cellular aggregate comprises a bioactive cell population described herein. In certain embodiments, the cellular aggregate comprises bioactive renal cells such as, for example, renal cell admixtures, enriched renal cell populations, and combinations of renal cell fractions and admixtures of renal cells with mesenchymal stem cells, endothelial progenitor cells, cells derived from the stromal vascular fraction of adipose, or any other non-renal cell population without limitation.

In certain embodiments, the bioactive renal cells of the disclosure may be cultured in 3D formats as described further herein. In some embodiments, the term “organoid” refers to an accumulation of cells, with a phenotype and/or function, that recapitulates aspects of native kidney. In some embodiments, organoids comprise mixed populations of cells, of a variety of lineages, which are typically found in vivo in a given tissue. In some embodiments, the organoids of this disclosure are formed in vitro, via any means, whereby the cells of the disclosure form aggregates, which in turn may form spheroids, organoids, or a combination thereof. Such aggregates, spheroids or organoids, in some embodiments, assume a structure consistent with a particular organ. In some embodiments, such aggregates, spheroids or organoids, express surface markers, which are typically expressed by cells of the particular organ. In some embodiments, such aggregates, spheroids or organoids, produce compounds or materials, which are typically expressed by cells of the particular organ. In certain embodiments, the cells of the disclosure may be cultured on natural substrates, e.g., gelatin. In certain embodiments, the cells of the disclosure may be cultured on synthetic substrates, e.g., PLGA.

### 3. Biomaterials

A variety of biomaterials may be combined with an active agent to provide the therapeutic formulations of the present disclosure. The biomaterials may be in any suitable shape (e.g., beads) or form (e.g., liquid, gel, etc.). As described in Bertram et al. U.S. Published Application 20070276507 (incorporated herein by reference in its entirety), polymeric matrices or scaffolds may be shaped into any number of desirable configurations to satisfy any number of overall system, geometry or space restrictions. In some embodiments, a biomaterial is in the form of a liquid suspension. In certain embodiments, the matrices or scaffolds of the present disclosure may be three-dimensional and shaped to conform to the dimensions and shapes of an organ or tissue structure. For example, in the use of the polymeric scaffold for treating kidney disease, tubular transport deficiency, or glomerular filtration deficiency, a three-dimensional (3-D) matrix may be used that recapitulates aspects or the entirety of native kidney tissue structure and organization as well as that of renal parenchyma.

A variety of differently shaped 3-D scaffolds may be used. Naturally, the polymeric matrix may be shaped in different sizes and shapes to conform to differently sized patients. The polymeric matrix may also be shaped in other ways to accommodate the special needs of the patient. In certain embodiments, the polymeric matrix or scaffold may be a biocompatible, porous polymeric scaffold. The scaffolds may be formed from a variety of synthetic or naturally-occurring materials including, but not limited to, open-cell polylactic acid (OPLA®), cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, collagens, gelatin, alginate, laminins, fibronectin, silk, elastin, alginate, hyaluronic acid, agarose, or copolymers or physical blends thereof. Scaffolding configurations may range from soft porous scaffolds to rigid, shape-holding porous scaffolds. In certain embodiments, a scaffold is configured as a liquid solution that is capable of becoming a hydrogel, e.g., hydrogel that is above a melting temperature.

In certain embodiments, the scaffold is derived from an existing kidney or other organ of human or animal origin, where the native cell population has been eliminated through application of detergent and/or other chemical agents and/or other enzymatic and/or physical methodologies known to those of ordinary skill in the art. In this embodiment, the native three dimensional structure of the source organ is retained together with all associated extracellular matrix components in their native, biologically active context. In certain embodiments, the scaffold is extracellular matrix derived from human or animal kidney or other organ. In certain embodiments, the configuration is assembled into a tissue-like structure through application of three dimensional bioprinting methodologies. In certain embodiments, the configuration is the liquid form of a solution that is capable of becoming a hydrogel.

Hydrogels may be formed from a variety of polymeric materials and are useful in a variety of biomedical applications. Hydrogels can be described physically as three-dimensional networks of hydrophilic polymers. Depending on the type of hydrogel, they contain varying percentages of water, but altogether do not dissolve in water. Despite their high water content, hydrogels are capable of additionally binding great volumes of liquid due to the presence of hydrophilic residues. Hydrogels swell extensively without changing their gelatinous structure. The basic physical features of a hydrogel can be specifically modified, according to the properties of the polymers used and a device used to administer the hydrogel.

The hydrogel material preferably does not induce an inflammatory response. Examples of other materials which can be used to form a hydrogel include (a) modified alginates, (b) polysaccharides (e.g. gellan gum and carrageenans) which gel by exposure to monovalent cations, (c) polysaccharides (e.g., hyaluronic acid) that are very viscous liquids or are thixotropic and form a gel over time by the slow evolution of structure, (d) gelatin or collagen, and (e) polymeric hydrogel precursors (e.g., polyethylene oxide-polypropylene glycol block copolymers and proteins). U.S. Pat. No. 6,224,893 B1 provides a detailed description of the various polymers, and the chemical properties of such polymers, that are suitable for making hydrogels in accordance with the present disclosure.

In a particular embodiment, the hydrogel used to formulate the biomaterials of the present disclosure is gelatin-based. Gelatin is a non-toxic, biodegradable and water-soluble protein derived from collagen, which is a major component of mesenchymal tissue extracellular matrix (ECM). Collagen is the main structural protein in the extracellular space in the various connective tissues in animal bodies. As the main component of connective tissue, it is the most abundant protein in mammals, making up from 25% to 35% of the whole-body protein content. Depending upon the degree of mineralization, collagen tissues may be rigid (bone), compliant (tendon), or have a gradient from rigid to compliant (cartilage). Collagen, in the form of elongated fibrils, is mostly found in fibrous tissues such as tendons, ligaments and skin. It is also abundant in corneas, cartilage, bones, blood vessels, the gut, intervertebral discs and the dentin in teeth. In muscle tissue, it serves as a major component of the endomysium. Collagen constitutes one to two percent of muscle tissue, and accounts for 6% of the weight of strong, tendinous muscles. Collagen occurs in many places throughout the body. Over 90% of the collagen in the human body, however, is type I.

To date, 28 types of collagen have been identified and described. They can be divided into several groups according to the structure they form: Fibrillar (Type I, II, III, V, XI). Non-fibrillar FACIT (Fibril Associated Collagens with Interrupted Triple Helices) (Type IX, XII, XIV, XVI, XIX). Short chain (Type VIII, X). Basement membrane (Type IV). Multiplexin (Multiple Triple Helix domains with Interruptions) (Type XV, XVIII). MACIT (Membrane Associated Collagens with Interrupted Triple Helices) (Type XIII, XVII). Other (Type VI, VII). The five most common types are: Type I: skin, tendon, vascular ligature, organs, bone (main component of the organic part of bone). Type II: cartilage (main collagenous component of cartilage) Type III: reticulate (main component of reticular fibers), commonly found alongside type I.Type IV: forms basal lamina, the epithelium-secreted layer of the basement membrane. Type V: cell surfaces, hair and placenta.

Gelatin retains informational signals including an arginine-glycine-aspartic acid (RGD) sequence, which promotes cell adhesion, proliferation and stem cell differentiation. A characteristic property of gelatin is that it exhibits Upper Critical Solution Temperature behavior (UCST). Above a specific temperature threshold of 40° C., gelatin can be dissolved in water by the formation of flexible, random single coils. Upon cooling, hydrogen bonding and Van der Waals interactions occur, resulting in the formation of triple helices. These collagen-like triple helices act as junction zones and thus trigger the sol-gel transition. Gelatin is widely used in pharmaceutical and medical applications.

In certain embodiments, the hydrogel used to formulate the injectable cell compositions herein is based on porcine gelatin, which may be sourced from porcine skin and is commercially available, for example from Nitta Gelatin NA Inc (NC, USA) or Gelita USA Inc. (IA, USA). Gelatin may be dissolved, for example, in Dulbecco's phosphate-buffered saline (DPBS) to form a thermally responsive hydrogel, which can gel and liquefy at different temperatures. In certain embodiments, the hydrogel used to formulate the injectable cell compositions herein is based on recombinant human or animal gelatin expressed and purified using methodologies known to those of ordinary skill in the art. In certain embodiments, an expression vector containing all or part of the cDNA for Type I, alpha I human collagen is expressed in the yeast Pichia pastoris. Other expression vector systems and organisms will be known to those of ordinary skill in the art. In a particular embodiment, the gelatin-based hydrogel of the present disclosure is liquid at and above room temperature (22-28° C.) and gels when cooled to refrigerated temperatures (2-8° C.).

Those of ordinary skill in the art will appreciate that other types of synthetic or naturally-occurring materials known in the art may be used to form scaffolds as described herein.

In certain embodiments, the biomaterial used in accordance with the present disclosure is comprised of hyaluronic acid (HA) in hydrogel form, containing HA molecules ranging in size from 5.1 kDA to >2×10⁵ kDa. HA may promote branching morphogenesis and three dimensional self-organization of associated bioactive cell populations. In certain embodiments, the biomaterial used in accordance with the present disclosure is comprised of hyaluronic acid in porous foam form, also containing HA molecules ranging in size from 5.1 kDA to >2×10⁵ kDa. In certain embodiments, the hydrogel is derived from, or contains extracellular matrix sourced from kidney or any other tissue or organ without limitation. In yet another embodiment, the biomaterial used in accordance with the present disclosure is comprised of a poly-lactic acid (PLA)-based foam, having an open-cell structure and pore size of about 50 microns to about 300 microns.

Temperature-Sensitive Biomaterials

The biomaterials described herein may also be designed or adapted to respond to certain external conditions, e.g., in vitro or in vivo. In certain embodiments, the biomaterials are temperature-sensitive (e.g., either in vitro or in vivo). In certain embodiments, the biomaterials are adapted to respond to exposure to enzymatic degradation (e.g., either in vitro or in vivo). The biomaterials' response to external conditions can be fine-tuned as described herein. Temperature sensitivity of the formulation described can be varied by adjusting the percentage of a biomaterial in the formulation. For example, the percentage of gelatin in a solution can be adjusted to modulate the temperature sensitivity of the gelatin in the final formulation (e.g., liquid, gel, beads, etc.). Alternatively, biomaterials may be chemically crosslinked to provide greater resistance to enzymatic degradation. For instance, a carbodiimide crosslinker may be used to chemically crosslink gelatin beads thereby providing a reduced susceptibility to endogenous enzymes.

In one aspect, the formulations described herein incorporate biomaterials having properties which create a favorable environment for the active agent, such as bioactive renal cells, to be administered to a subject. In certain embodiments, the formulation contains a first biomaterial that provides a favorable environment from the time the active agent is formulated with the biomaterial up until the point of administration to the subject. In one other embodiment, the favorable environment concerns the advantages of having bioactive cells suspended in a substantially solid state versus cells in a fluid (as described herein) prior to administration to a subject. In certain embodiments, the first biomaterial is a temperature-sensitive biomaterial. The temperature-sensitive biomaterial may have (i) a substantially solid state at about 8° C. or below, and (ii) a substantially liquid state at ambient temperature or above. In certain embodiments, the ambient temperature is about room temperature.

In certain embodiments, the biomaterial is a temperature-sensitive biomaterial that can maintain at least two different phases or states depending on temperature. The biomaterial is capable of maintaining a first state at a first temperature, a second state at a second temperature, and/or a third state at a third temperature. The first, second or third state may be a substantially solid, a substantially liquid, or a substantially semi-solid or semi-liquid state. In certain embodiments, the biomaterial has a first state at a first temperature and a second state at a second temperature, wherein the first temperature is lower than the second temperature.

In one other embodiment, the state of the temperature-sensitive biomaterial is a substantially solid state at a temperature of about 8° C. or below. In certain embodiments, the substantially solid state is maintained at about 1° C., about 2° C., about 3° C., about 4° C., about 5° C., about 6° C., about 7° C., or about 8° C. In certain embodiments, the substantially solid state has the form of a gel. In certain embodiments, the state of the temperature-sensitive biomaterial is a substantially liquid state at ambient temperature or above. In certain embodiments, the substantially liquid state is maintained at about 25° C., about 25.5° C., about 26° C., about 26.5° C., about 27° C., about 27.5° C., about 28° C., about 28.5° C., about 29° C., about 29.5° C., about 30° C., about 31° C., about 32° C., about 33° C., about 34° C., about 35° C., about 36° C., or about 37° C. In certain embodiments, the ambient temperature is about room temperature.

In certain embodiments, the state of the temperature-sensitive biomaterial is a substantially solid state at a temperature of about ambient temperature or below. In certain embodiments, the ambient temperature is about room temperature. In certain embodiments, the substantially solid state is maintained at about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In certain embodiments, the substantially solid state has the form of a bead. In certain embodiments, the state of the temperature-sensitive biomaterial is a substantially liquid state at a temperature of about 37° C. or above. In one other embodiment, the substantially solid state is maintained at about 37° C., about 38° C., about 39° C., or about 40° C.

The temperature-sensitive biomaterials may be provided in the form of a solution, in the form of a solid, in the form of beads, or in other suitable forms described herein and/or known to those of ordinary skill in the art. The cell populations and preparations described herein may be coated with, deposited on, embedded in, attached to, seeded, suspended in, or entrapped in a temperature-sensitive biomaterial. In certain embodiments, the cell populations described herein may be assembled as three dimensional cellular aggregrates or organoids or three dimensional tubular structures prior to complexing with the temperature-sensitive biomaterial or may be assembled as such upon complexing with the temperature-sensitive biomaterial. Alternatively, the temperature-sensitive biomaterial may be provided without any cells, such as, for example in the form of spacer beads. In this embodiment, the temperature sensitive biomaterial functions in a purely passive role to create space within the target organ for regenerative bioactivity, for example, angiogenesis or infiltration and migration of host cell populations.

In certain embodiments, the temperature-sensitive biomaterial has a transitional state between a first state and a second state. In certain embodiments, the transitional state is a solid-to-liquid transitional state between a temperature of about 8° C. and about ambient temperature. In certain embodiments, the ambient temperature is about room temperature. In one other embodiment, the solid-to-liquid transitional state occurs at one or more temperatures of about 8° C., about 9° C., about 10° C., about 11° C., about 12° C., about 13° C., about 14° C., about 15° C., about 16° C., about 17° C., and about 18° C.

The temperature-sensitive biomaterials have a certain viscosity at a given temperature measured in centipoise (cP). In certain embodiments, the biomaterial has a viscosity at 25° C. of about 1 cP to about 5 cP, about 1.1 cP to about 4.5 cP, about 1.2 cP to about 4 cP, about 1.3 cP to about 3.5 cP, about 1.4 cP to about 3.5 cP, about 1.5 cP to about 3 cP, about 1.55 cP to about 2.5 cP, or about 1.6 cP to about 2 cP. In certain embodiments, the biomaterial has a viscosity at 37° C. of about 1.0 cP to about 1.15 cP. The viscosity at 37° C. may be about 1.0 cP, about 1.01 cP, about 1.02 cP, about 1.03 cP, about 1.04 cP, about 1.05 cP, about 1.06 cP, about 1.07 cP, about 1.08 cP, about 1.09 cP, about 1.10 cP, about 1.11 cP, about 1.12 cP, about 1.13 cP, about 1.14 cP, or about 1.15 cP. In one other embodiment, the biomaterial is a gelatin solution. The gelatin is present at about 0.5%, about 0.55%, about 0.6%, about 0.65%, about 0.7%, about 0.75%, about 0.8%, about 0.85%, about 0.9%, about 0.95% or about 1%, (w/v) in the solution. In one example, the biomaterial is a 0.75% (w/v) gelatin solution in PBS. In certain embodiments, the 0.75% (w/v) solution has a viscosity at 25° C. of about 1.6 cP to about 2 cP. In certain embodiments, the 0.75% (w/v) solution has a viscosity at 37° C. of about 1.07 cP to about 1.08 cP. The gelatin solution may be provided in PBS, DMEM, or another suitable solvent.

In another aspect, the formulation contains bioactive cells combined with a second biomaterial that provides a favorable environment for the combined cells from the time of formulation up until a point after administration to the subject. In certain embodiments, the favorable environment provided by the second biomaterial concerns the advantages of administering cells in a biomaterial that retains structural integrity up until the point of administration to a subject and for a period of time after administration. In certain embodiments, the structural integrity of the second biomaterial following implantation is minutes, hours, days, or weeks. In certain embodiments, the structural integrity is less than one month, less than one week, less than one day, or less than one hour. The relatively short term structural integrity provides a formulation that can deliver the active agent and biomaterial to a target location in a tissue or organ with controlled handling, placement or dispersion without being a hindrance or barrier to the interaction of the incorporated elements with the tissue or organ into which it was placed.

In certain embodiments, the second biomaterial is a temperature-sensitive biomaterial that has a different sensitivity than the first biomaterial. The second biomaterial may have (i) a substantially solid state at about ambient temperature or below, and (ii) a substantially liquid state at about 37° C. or above. In certain embodiments, the ambient temperature is about room temperature.

In certain embodiments, the second biomaterial is crosslinked beads. The crosslinked beads may have finely tunable in vivo residence times depending on the degree of crosslinking, as described herein. In certain embodiments, the crosslinked beads comprise bioactive cells and are resistant to enzymatic degradation as described herein. The formulations of the present disclosure may include the first biomaterial combined with an active agent, e.g., bioactive cells, with or without a second biomaterial combined with an active agent, e.g., bioactive cells. Where a formulation includes a second biomaterial, it may be a temperature sensitive bead and/or a crosslinked bead.

In another aspect, the present disclosure provides formulations that contain biomaterials which degrade over a period of time on the order of minutes, hours, or days. This is in contrast to a large body of work focusing on the implantation of solid materials that then slowly degrade over days, weeks, or months. In certain embodiments, the biomaterial has one or more of the following characteristics: biocompatibility, biodegradeability/bioresorbablity, a substantially solid state prior to and during implantation into a subject, loss of structural integrity (substantially solid state) after implantation, and cytocompatible environment to support cellular viability and proliferation. The biomaterial's ability to keep implanted particles spaced out during implantation enhances native tissue ingrowth. The biomaterial also facilitates implantation of solid formulations. The biomaterial provides for localization of the formulation described herein since insertion of a solid unit helps prevent the delivered materials from dispersing within the tissue during implantation. For cell-based formulations, a solid biomaterial also improves stability and viability of anchorage dependent cells compared to cells suspended in a fluid. However, the short duration of the structural integrity means that soon after implantation, the biomaterial does not provide a significant barrier to tissue ingrowth or integration of the delivered cells/materials with host tissue.

In one aspect, the present disclosure provides formulations that contain biomaterials which are implanted in a substantially solid form and then liquefy/melt or otherwise lose structural integrity following implantation into the body. This is in contrast to the significant body of work focusing on the use of materials that can be injected as a liquid, which then solidify in the body.

Biocompatible Beads

In one other aspect, the formulation includes a temperature-sensitive biomaterial described herein and a population of biocompatible beads containing a biomaterial. In certain embodiments, the beads are crosslinked. Crosslinking may be achieved using any suitable crosslinking agent known to those of ordinary skill in the art, such as, for example, carbodiimides; aldehydes (e.g. furfural, acrolein, formaldehyde, glutaraldehyde, glyceryl aldehyde), succinimide-based crosslinkers {Bis(sulfosuccinimidyl) suberate (BS3), Disuccinimidyl glutarate (DSG), Disuccinimidyl suberate (DSS), Dithiobis(succinimidyl propionate), Ethylene glycolbis(sulfosuccinimidylsuccinate), Ethylene glycolbis(succinimidylsuccinate) (EGS), Bis(Sulfosuccinimidyl) glutarate (BS2G), Disuccinimidyl tartrate (DST)}; epoxides (Ethylene glycol diglycidyl ether, 1,4 Butanediol diglycidyl ether); saccharides (glucose and aldose sugars); sulfonic acids and p-toluene sulfonic acid; carbonyldiimidazole; genipin; imines; ketones; diphenylphosphorylazide (DDPA); terephthaloyl chloride; cerium (III) nitrate hexahydrate; microbial transglutaminase; and hydrogen peroxide. Those of ordinary skill in the art will appreciate other suitable crosslinking agents and crosslinking methods for use in accordance with the present disclosure.

In certain embodiments, the beads are carbodiimide-crosslinked beads. The carbodiimide-crosslinked beads may be crosslinked with a carbodiimide selected from the group consisting of 1-Ethyl-3-[3-dimethylaminopropyl] carbodiimide hydrochloride (EDC), DCC—N,N′-dicyclohexylcarbodiimide (DCC), and N,N′-Diisopropylcarbodiimide (DIPC). Beads treated with lower concentration of EDC were expected to have a higher number of free primary amines, while samples treated with high concentrations of crosslinker would have most of the primary amines engaged in amide bonds. The intensity of the orange color developed by the covalent bonding between the primary amine and picrylsulfonic acid, detectable spectrophotometrically at 335 nm, is proportional to the number of primary amines present in the sample. When normalized per milligram of protein present in the sample, an inverse correlation between the number of free amines present and the initial concentration of EDC used for crosslinking can be observed. This result is indicative of differential bead crosslinking, dictated by the amount of carbodiimide used in the reaction. In general, crosslinked beads exhibit a reduced number of free primary amines as compared to non-crosslinked beads.

The crosslinked beads have a reduced susceptibility to enzymatic degradation as compared to non-crosslinked biocompatible beads, thereby providing beads with finely tunable in vivo residence times. For example, the crosslinked beads are resistant to endogenous enzymes, such as collagenases. The provision of crosslinked beads is part of a delivery system that facilitate one or more of: (a) delivery of attached cells to the desired sites and creation of space for regeneration and ingrowth of native tissue and vascular supply; (b) ability to persist at the site long enough to allow cells to establish, function, remodel their microenvironment and secrete their own extracellular matrix (ECM); (c) promotion of integration of the transplanted cells with the surrounding tissue; (d) ability to implant cells in a substantially solid form; (e) short term structural integrity that does not provide a significant barrier to tissue ingrowth, de novo angiogenesis or integration of delivered cells/materials with the host tissue; (0 localized in vivo delivery in a substantially solid form thereby preventing dispersion of cells within the tissue during implantation; (g) improved stability and viability of anchorage dependent cells compared to cells suspended in a fluid; and (h) biphasic release profile when cells are delivered 1) in a substantially solid form (e.g., attached to beads), and 2) in a substantially liquid form (e.g., suspended in a fluid); i) recapitulation and mimicry of the three dimensional biological niche or renal parenchyma from which these bioactive cell populations were derived.

In certain embodiments, the present disclosure provides crosslinked beads containing gelatin. Non-crosslinked gelatin beads are not suitable for a bioactive cell formulation because they rapidly lose integrity and cells dissipate from the injection site. In contrast, highly crosslinked gelatin beads may persist too long at the injection site and may hinder the de-novo ECM secretion, cell integration, angiogenesis and tissue regeneration. The present disclosure allows for the in vivo residence time of the crosslinked beads to be finely tuned. In order to tailor the biodegradability of biomaterials, different crosslinker concentrations of carbodiimide are used while the overall reaction conditions were kept constant for all samples. For example, the enzymatic susceptibility of carbodiimide-crosslinked beads can be finely tuned by varying the concentration of crosslinking agent from about zero to about 1M. In some embodiments, the concentration is about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 55 mM, about 60 mM, about 65 mM, about 70 mM, about 75 mM, about 80 mM, about 85 mM, about 90 mM, about 95 mM, or about 100 mM. The crosslinker concentration may also be about 0.15 M, about 0.2 M, about 0.25 M, about 0.3 M, about 0.35 M, about 0.4 M, about 0.45 M, about 0.5 M, about 0.55 M, about 0.6 M, about 0.65 M, about 0.7 M, about 0.75 M, about 0.8 M, about 0.85 M, about 0.9 M, about 0.95 M, or about 1 M. In certain embodiments, the crosslinking agent is 1-Ethyl-3-[3-dimethylaminopropyl] carbodiimide hydrochloride (EDC). In certain embodiments, the EDC-crosslinked beads are gelatin beads. The % degradation of the beads can be finely tuned depending upon the concentration of crosslinking agent. In certain embodiments, gelatin beads may be mixed with beads or microparticles other than gelatin (for example, without limitation, alginate or HA) to additionally facilitate the potency of the bioactive cell population being delivered.

Crosslinked beads may have certain characteristics that favor the seeding, attachment, or encapsulation of bioactive cell populations. For example, the beads may have a porous surface and/or may be substantially hollow. The presence of pores provides an increased cell attachment surface allowing for a greater number of cells to attach as compared to a non-porous or smooth surface. In addition, the pore structure can support host tissue integration with the porous beads supporting the formation of de novo tissue. The beads have a size distribution that can be fitted to a Weibull plot corresponding to the general particle distribution pattern. In certain embodiments, the crosslinked beads have an average diameter of less than about 120 μm, about 115 μm, about 110 μm, about 109 μm, about 108 μm, about 107 μm, about 106 μm, about 105 μm, about 104 μm, about 103 μm, about 102 μm, about 101 μm, about 100 μm, about 99 μm, about 98 μm, about 97 μm, about 96 μm, about 95 μm, about 94 μm, about 93 μm, about 92 μm, about 91 μm, or about 90 μm. The characteristics of the crosslinked beads vary depending upon the casting process. For instance, a process in which a stream of air is used to aerosolize a liquid gelatin solution and spray it into liquid nitrogen with a thin layer chromatography reagent sprayer (ACE Glassware) is used to provide beads having the afore-mentioned characteristics. Those of skill in the art will appreciate that modulating the parameters of the casting process provides the opportunity to tailor different characteristics of the beads, e.g., different size distributions. In certain embodiments, the microtopography, surface and internal characteristics of the beads may be further modified to facilitate cell attachment.

The cytocompatibility of the crosslinked beads is assessed in vitro prior to formulation using cell culture techniques in which beads are cultured with cells that correspond to the final bioactive cell formulation. For instance, the beads are cultured with primary renal cells prior to preparation of a bioactive renal cell formulation and live/dead cell assays are used to confirm cytocompatibility. In addition to cellular viability, specific functional tests to measure cellular metabolic activity, secretion of certain key cytokines and growth factors and exosomes and the expression of certain key protein and nucleic acid markers including miRNAs associated with functionally bioactive renal cell populations are well known to those of ordinary skill in the art and are additionally used to confirm cell potency upon formulation with crosslinked beads.

In certain formulations, the biocompatible crosslinked beads are combined with a temperature-sensitive biomaterial in solution at about 5% (w/w) to about 15% (w/w) of the volume of the solution. The crosslinked beads may be present at about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), about 10% (w/w), about 10.5% (w/w), about 11% (w/w), about 11.5% (w/w), about 12% (w/w), about 12.5% (w/w), about 13% (w/w), about 13.5% (w/w), about 14% (w/w), about 14.5% (w/w), or about 15% (w/w) of the volume of the solution.

In another aspect, the present disclosure provides formulations that contain biomaterials which degrade over a period time on the order of minutes, hours, or days. This is in contrast to a large body of work focusing on the implantation of solid materials that then slowly degrade over days, weeks, or months.

In another aspect, the present disclosure provides formulations having biocompatible crosslinked beads seeded with bioactive cells together with a delivery matrix. In certain embodiments, the delivery matrix has one or more of the following characteristics: biocompatibility, biodegradeability/bioresorbability, a substantially solid state prior to and during implantation into a subject, loss of structural integrity (substantially solid state) after implantation, and a cytocompatible environment to support cellular viability. The delivery matrix's ability to keep implanted particles (e.g., crosslinked beads) spaced out during implantation enhances native tissue ingrowth. If the delivery matrix is absent, then compaction of cellularized beads during implantation can lead to inadequate room for sufficient tissue ingrowth. The delivery matrix facilitates implantation of solid formulations. In addition, the short duration of the structural integrity means that soon after implantation, the matrix does not provide a significant barrier to tissue ingrowth, de novo angiogenesis or integration of the delivered cells/materials with host tissue. The delivery matrix provides for localization of the formulation described herein since insertion of a solid unit helps prevent the delivered materials from dispersing within the tissue during implantation. In certain embodiments, application of a delivery matrix as described herein helps prevent rapid loss of implanted cells through urination upon delivery to the renal parenchyme. For cell-based formulations, a solid delivery matrix improves stability and viability of anchorage dependent cells compared to cells suspended in a fluid.

In certain embodiments, the delivery matrix is a population of biocompatible beads that is not seeded with cells. In certain embodiments, the unseeded beads are dispersed throughout and in between the individual cell-seeded beads. The unseeded beads act as “spacer beads” between the cell-seeded beads prior to and immediately after transplantation. The spacer beads contain a temperature-sensitive biomaterial having a substantially solid state at a first temperature and a substantially liquid state at a second temperature, wherein the first temperature is lower than the second temperature. For example, the spacer beads contain a biomaterial having a substantially solid state at about ambient temperature or below and a substantially liquid state at about 37° C., such as that described herein. In certain embodiments, the ambient temperature is about room temperature. In certain embodiments, the biomaterial is a gelatin solution. The gelatin solution is present at about 4%, about 4.5%, about 5%, about 5.5%, about 6%, about 6.5%, about 7%, about 7.5%, about 8%, about 8.5%, about 9%, about 9.5%, about 10%, about 10.5%, or about 11%, (w/v). The gelatin solution may be provided in PBS, cell culture media (e.g., DMEM), or another suitable solvent. In certain embodiments, the biomaterial is hyaluronic acid. In certain embodiments, the biomaterial is decellularized extracellular matrix sourced from human or animal kidney which may be further reconstituted as a hydrogel.

In one aspect, the present disclosure provides formulations that contain biomaterials which are implanted in a substantially solid form (e.g., spacer beads) and then liquefy/melt or otherwise lose structural integrity following implantation into the body. This is in contrast to the significant body of work focusing on the use of materials that can be injected as a liquid, which then solidify in the body.

The temperature-sensitivity of spacer beads can be assessed in vitro prior to formulation. Spacer beads can be labeled and mixed with unlabeled non-temperature-sensitive beads. The mixture is then incubated at 37° C. to observe changes in physical transition. The loss of shape of the labeled temperature-sensitive beads at the higher temperature is observed over time. For example, temperature-sensitive gelatin beads may be made with Alcian blue dye to serve as a marker of physical transition. The blue gelatin beads are mixed with crosslinked beads (white), loaded into a catheter, then extruded and incubated in 1×PBS, pH 7.4, at 37° C. The loss of shape of the blue gelatin beads is followed microscopically at different time points. Changes in the physical state of the blue gelatin beads are visible after 30 min becoming more pronounced with prolonged incubation times. The beads do not completely dissipate because of the viscosity of the material.

Modified Release Formulations

In one aspect, the formulations of the present disclosure are provided as modified release formulations. In general, the modified release is characterized by an initial release of a first active agent upon administration followed by at least one additional, subsequent release of a second active agent. The first and second active agents may be the same or they may be different. In certain embodiments, the formulations provide modified release through multiple components in the same formulation. In certain embodiments, the modified release formulation contains an active agent as part of a first component that allows the active agent to move freely throughout the volume of the formulation, thereby permitting immediate release at the target site upon administration. The first component may be a temperature-sensitive biomaterial having a substantially liquid phase and a substantially solid phase, wherein the first component is in a substantially liquid phase at the time of administration. In certain embodiments, the active agent is in the substantially liquid phase such that it is substantially free to move throughout the volume of the formulation, and therefore is immediately released to the target site upon administration.

In certain embodiments, the modified release formulation has an active agent as part of a second component in which the active agent is attached to, deposited on, coated with, embedded in, seeded upon, or entrapped in the second component, which persists before and after administration to the target site. The second component contains structural elements with which the active agent is able to associate with, thereby preventing immediate release of the active agent from the second component at the time of administration. For example, the second component is provided in a substantially solid form, e.g., biocompatible beads, which may be crosslinked to prevent or delay in vivo enzymatic degradation. In certain embodiments, the active agent in the substantially solid phase retains its structural integrity within the formulation before and after administration and therefore it does not immediately release the active agent to the target site upon administration. Suitable carriers for modified release formulations have been described herein but those of ordinary skill in the art will appreciate other carriers that are appropriate for use in the present disclosure.

In certain embodiments, the formulation provides an initial rapid delivery/release of delivered elements, including cells, nanoparticles, therapeutic molecules, etc. followed by a later delayed release of elements. In certain embodiments, the formulation provides an initial rapid delivery/release of exosomes, miRNA and other bioactive nucleic acid or protein molecules that are soluble and are secreted, bioactive products sourced from renal or other cell populations. Other molecules or therapeutic agents associated with regenerative bioactivity will be appreciated by those of ordinary skill in the art. The formulations of the present disclosure can be designed for such biphasic release profile where the agent to be delivered is provided in both an unattached form (e.g., cells in a solution) and an attached form (e.g., cells together with beads or another suitable carrier). Upon initial administration, the unencumbered agent is provided immediately to the site of delivery while release of the encumbered agent is delayed until structural integrity of the carrier (e.g., beads) fails at which point the previously attached agent is released. As discussed below, other suitable mechanisms of release will be appreciated by those of ordinary skill in the art.

The time delay for release can be adjusted based upon the nature of the active agent. For example, the time delay for release in a bioactive cell formulation may be on the order of seconds, minutes, hours, or days. In some circumstances, a delay on the order of weeks may be appropriate. For other active agents, such as small or large molecules, the time delay for release in a formulation may be on the order of seconds, minutes, hours, days, weeks, or months. It is also possible for the formulation to contain different biomaterials that provide different time delay release profiles. For example, a first biomaterial with a first active agent may have a first release time and a second biomaterial with a second active agent may have a second release time. The first and second active agent may be the same or different.

As discussed herein, the time period of delayed release may generally correspond to the time period for loss of structural integrity of a biomaterial. However, those of ordinary skill in the art will appreciate other mechanisms of delayed release. For example, an active agent may be continually released over time independent of the degradation time of any particular biomaterial, e.g., diffusion of a drug from a polymeric matrix. In addition, bioactive cells can migrate away from a formulation containing a biomaterial and the bioactive cells to native tissue. In certain embodiments, bioactive cells migrate off of a biomaterial, e.g., a bead, to the native tissue. In one embodimemt, bioactive cells migrate off a biomaterial to the native tissue and induce secretion of growth factors, cytokines, exosomes, miRNA and other nucleic acids and proteins associated with regenerative bioactivity. In certain embodiments, exosomes and other extracellular vesicles, as well as miRNA, other bioactive nucleic acids and proteins migrate off of a biomaterial. In yet another embodiment, bioactive cells migrate off a biomaterial to the native tissue and mediate mobilization of host stem and progenitor cells that then migrate or home towards the injury or disease location.

Biodegradable, biocompatible polymers can be used, such as ethylene vinyl acetate, polyanhydrides, polyglycolic acid, collagen, polyorthoesters, and polylactic acid. Prolonged absorption of injectable formulations can be brought about by including in the formulation an agent that delays absorption, for example, monostearate salts and gelatin. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. Additional methods applicable to the controlled or extended release of polypeptide agents are described, for example, in U.S. Pat. Nos. 6,306,406 and 6,346,274, as well as, for example, in U.S. Patent Application Nos. US20020182254 and US20020051808, all of which are incorporated herein by reference.

### 4. Bioactive Cell Formulations

The bioactive cell formulations described herein contain implantable constructs made from the above-referenced biomaterials having the bioactive renal cells described herein for the treatment of kidney disease in a subject in need. In certain embodiments, the construct is made up of a biocompatible material or biomaterial, scaffold or matrix composed of one or more synthetic or naturally-occurring biocompatible materials and one or more cell populations or admixtures of cells described herein deposited on or embedded in a surface of the scaffold by attachment and/or entrapment. In certain embodiments, the construct is made up of a biomaterial and one or more cell populations or admixtures of cells described herein coated with, deposited on, deposited in, attached to, entrapped in, embedded in, seeded, or combined with the biomaterial component(s). Any of the cell populations described herein, including enriched cell populations or admixtures thereof, may be used in combination with a matrix to form a construct. In certain embodiments, the bioactive cell formulation is made up of a biocompatible material or biomaterial and an SRC population described herein. In certain embodiments, the bioactive cell formulation is made up of a biocompatible material or biomaterial and an admixture of the SRC cell population described herein with another cell population, that may include, without limitation, endothelial progenitor cells, mesenchymal stem cells and cells derived from the stromal vascular fraction of adipose.

Neo-Kidney Augment Description and Composition

In certain embodiments, the bioactive cell formulation is a Neo-Kidney Augment (NKA), which is an injectable product composed of autologous, selected renal cells (SRC) formulated in a Biomaterial (gelatin-based hydrogel). In one aspect, autologous SRC are obtained from isolation and expansion of renal cells from the patient's renal cortical tissue via a kidney biopsy and selection by centrifugation of the expanded renal cells across a density boundary, barrier, or interface. In certain embodiments, autologous SRC are obtained from isolation and expansion of renal cells from the patient's renal cortical tissue via a kidney biopsy and selection of the expanded renal cells over a continuous or discontinuous single step or multistep density gradient. SRC are composed primarily of renal tubular epithelial cells which are well known for their regenerative potential (Humphreys et al. (2008) Intrinsic epithelial cells repair the kidney after injury. Cell Stem Cell. 2(3):284-91). Other parenchymal (vascular) and stromal (collecting duct) cells may be sparsely present in the autologous SRC population. Injection of SRC into recipient kidneys has resulted in significant improvement in animal survival, urine concentration and filtration functions in preclinical studies. However, SRC have limited shelf life and stability. Formulation of SRC in a gelatin-based hydrogel biomaterial provides enhanced stability of the cells thus extending product shelf life, improved stability of NKA during transport and delivery of NKA into the kidney cortex for clinical utility.

In another aspect, NKA is manufactured by first obtaining renal cortical tissue from the donor/recipient using a standard-of-clinical-care kidney biopsy procedure. Renal cells are isolated from the kidney tissue by enzymatic digestion and expanded using standard cell culture techniques. Cell culture medium is designed to expand primary renal cells and does not contain any differentiation factors. Harvested renal cells are subjected to separation across a density boundary or interface or density gradient to obtain SRC.

Temperature-Sensitive Formulations

One aspect of the disclosure further provides a formulation made up of biomaterials designed or adapted to respond to external conditions as described herein. As a result, the nature of the association of the bioactive cell population with the biomaterial in a construct will change depending upon the external conditions. For example, a cell population's association with a temperature-sensitive biomaterial varies with temperature. In certain embodiments, the construct contains a bioactive renal cell population and biomaterial having a substantially solid state at about 8° C. or lower and a substantially liquid state at about ambient temperature or above, wherein the cell population is suspended in the biomaterial at about 8° C. or lower. However, the cell population is substantially free to move throughout the volume of the biomaterial at about ambient temperature or above. Having the cell population suspended in the substantially solid phase at a lower temperature provides stability advantages for the cells, such as for anchorage-dependent cells, as compared to cells in a fluid. Moreover, having cells suspended in the substantially solid state provides one or more of the following benefits: i) prevents settling of the cells, ii) allows the cells to remain anchored to the biomaterial in a suspended state; iii) allows the cells to remain more uniformly dispersed throughout the volume of the biomaterial; iv) prevents the formation of cell aggregates; and v) provides better protection for the cells during storage and transportation of the formulation. A formulation that can retain such features leading up to the administration to a subject is advantageous at least because the overall health of the cells in the formulation will be better and a more uniform and consistent dosage of cells will be administered.

In a preferred embodiment, the gelatin-based hydrogel biomaterial used to formulate SRC into NKA is a porcine gelatin dissolved in buffer to form a thermally responsive hydrogel. This hydrogel is fluid at room temperature but gels when cooled to refrigerated temperature (2-8° C.). SRC are formulated with the hydrogel to obtain NKA. NKA is gelled by cooling and is shipped to the clinic under refrigerated temperature (2-8° C.). NKA has a shelf life of 3 days. At the clinical site, the product is warmed to room temperature before injecting into the patient's kidney. NKA is implanted into the kidney cortex using a needle and syringe suitable for delivery of NKA via a percutaneous or laparoscopic procedure. In certain embodiments, the hydrogel is derived from gelatin or another extracellular matrix protein of recombinant origin. In certain embodiments, the hydrogel is derived from extracellular matrix sourced from kidney or another tissue or organ. In certain embodiments, the hydrogel is derived from a recombinant extracellular matrix protein. In certain embodiments, the hydrogel comprises gelatin derived from recombinant collagen (i.e., recombinant gelatin).

Manufacturing Process

In certain embodiments, the manufacturing process for the bioactive cell formulations is designed to deliver a product in approximately four weeks from patient biopsy to product implant. Inherent patient-to-patient tissue variability poses a challenge to deliver product on a fixed implant schedule. Expanded renal cells are routinely cryopreserved during cell expansion to accommodate for this patient-dependent variation in cell expansion. Cryopreserved renal cells provide a continuing source of cells in the event that another treatment is needed (e.g., delay due to patient sickness, unforeseen process events, etc.) and to manufacture multiple doses for re-implantation, as required.

For embodiments where the bioactive cell formulation is composed of autologous, homologous cells formulated in a biomaterial (gelatin-based hydrogel), the final composition may be about 20×10⁶ cells per mL to about 200×10⁶ cells per mL in a gelatin solution with Dulbecco's Phosphate Buffered Saline (DPBS). In some embodiments, the number of cells per mL of product is about 20×10⁶ cells per mL, about 40×10⁶ cells per mL, about 60×10⁶ cells per mL, about 100×10⁶ cells per mL, about 120×10⁶ cells per mL, about 140×10⁶ cells per mL, about 160×10⁶ cells per mL, about 180×10⁶ cells per mL, or about 200×10⁶ cells per mL. In some embodiments, the gelatin is present at about 0.5%, about 0.55%, about 0.6%, about 0.65%, about 0.7%, about 0.75%, about 0.8%, about 0.85%, about 0.9%, about 0.95% or about 1%, (w/v) in the solution. In one example, the biomaterial is a 0.88% (w/v) gelatin solution in DPBS.

In a preferred embodiment, NKA is presented in a sterile, single-use 10 mL syringe. The final volume is calculated from the concentration of 100×10⁶ SRC/mL of NKA and the target dose of 3.0×10⁶ SRC/g kidney weight (estimated by MRI). Dosage may also be determined by the surgeon at the time of injection based on the patient's kidney weight.

This approach to developing NKA was based on extensive scientific evaluation of the active biological component, SRC (Bruce et al. (2011) Exposure of Cultured Human Renal Cells Induces Mediators of cell migration and attachment and facilitates the repair of tubular cell monolayers in vitro. Experimental Biology, Washington, D.C., available at www.regenmedtx.com/wp-content/uploads/2015/06/Bruce-EB2011-podium_compressed_Final-AB.pdf; Ilagan et al. (2010a) Exosomes derived from primary renal cells contain microRNAs that can potentially drive therapeutically-relevant outcomes in models of chronic kidney disease. TERMIS Conference, Orlando, Fla.; Ilagan et al. (2010b) Secreted Factors from Bioactive Kidney Cells Attenuate NF-kappa-B. TERMIS Conference, Orlando, Fla. available at www.regenmedtx.com/wp-content/uploads/2015/06/Ilagan-2010-TERMIS-poster-FINAL.pdf; Ilagan et al. (2009) Characterization of primary adult canine renal cells (CRC) in a three-dimensional (3D) culture system permissive for ex vivo nephrogenesis. KIDSTEM Conference, Liverpool, England, UK; Kelley et al. (2012) A Population of Selected Renal Cells Augments Renal Function and Extends Survival in the ZSF1 model of Progressive Diabetic Nephropathy. Cell Transplant 22(6), 1023-1039; Kelley et al. (2011) Intra-renal Transplantation of Bioactive Renal Cells Preserves Renal Functions and Extends Survival in the ZSF1 model of Progressive Diabetic Nephropathy. ADA Conference, San Diego, Calif., available at www.regenmedtx.com/wp-content/uploads/2015/06/ADA-2011-rwk Tengion-FINAL.pdf; Kelley et al. (2010a) A tubular cell-enriched subpopulation of primary renal cells improves survival and augments kidney function in a rodent model of chronic kidney disease. Am J Physiol Renal Physiol. 299(5), F1026-1039; Kelley et al. (2010b) Bioactive Renal Cells Augment Kidney Function In a Rodent Model Of Chronic Kidney Disease. ISCT Conference, Philadelphia, Pa. available at www.regenmedtx.com/wp-content/uploads/2015/06/Kelley-2010-ISCT-podium-FINAL.pdf; Kelley et al. (2008) Enhanced renal cell function in dynamic 3D culture system. KIDSTEM Conference, Liverpool, England, UK available at www.regenmedtx.com/wp-content/uploads/2015/06/Kelley-2008-KIDSTEM-poster-SEP2008_v1.pdf; Kelley et al. (2010c) Bioactive Renal Cells Augment Renal Function in the ZSF1 model of Diabetic Nephropathy. TERMIS Conference, Orlando, Fla. available at www.regenmedtx.com/wp-content/uploads/2015/06/Kelley-2010-TERMIS-FINAL.pdf; Presnell et al. (2010) Isolation, Characterization, and Expansion (ICE) methods for Defined Primary Renal Cell Populations from Rodent, Canine, and Human Normal and Diseased Kidneys. Tissue Engineering Part C Methods. 17(3):261-273; Presnell et al. (2009) Isolation and characterization of bioresponsive renal cells from human and large mammal with chronic renal failure. Experimental Biology, New Orleans, La. available at www.regenmedtx.com/wp-content/uploads/2015/06/Presnell-EB-poster-APR2009.pdf; Wallace et al. (2010) Quantitative Ex Vivo Characterization of Human Renal Cell Population Dynamics via High-Content Image-Based Analysis (HCA). ISCT Conference, Philadelphia, Pa. available at www.regenmedtx.com/wp-content/uploads/2015/06/Wallace-2010-ISCT-podium-FINAL.pdf; Yamaleyeva et al. (2010) Primary Human Kidney Cell Cultures Containing Erythropoietin-Producing Cells Improve Renal Injury. TERMIS Conference, Orlando, Fla.). In certain embodiments, SRC are an autologous, homologous cell population naturally involved in renal repair and regeneration. In a series of nonclinical pharmacology, physiology and mechanistic-biology studies, the characteristics of SRC were defined and the ability to delay the progression of CKD by augmenting renal structure and function has been demonstrated (Presnell et al. WO/2010/056328 and Ilagan et al. PCT/US2011/036347).

A total number of cells may be selected for the formulation and the volume of the formulation may be adjusted to reach the proper dosage. In some embodiments, the formulation may contain a dosage of cells to a subject that is a single dosage or a single dosage plus additional dosages. In certain embodiments, the dosages may be provided by way of a construct as described herein. The therapeutically effective amount of the bioactive renal cell populations or admixtures of renal cell populations described herein can range from the maximum number of cells that is safely received by the subject to the minimum number of cells necessary for treatment of kidney disease, e.g., stabilization, reduced rate-of-decline, or improvement of one or more kidney functions.

The therapeutically effective amount of the bioactive renal cell populations or admixtures thereof described herein can also be suspended in a pharmaceutically acceptable carrier or excipient. Such a carrier includes, but is not limited to basal culture medium plus 1% serum albumin, saline, buffered saline, dextrose, water, collagen, alginate, hyaluronic acid, fibrin glue, polyethyleneglycol, polyvinylalcohol, carboxymethylcellulose and combinations thereof. The formulation should suit the mode of administration.

The bioactive renal cell preparation(s), or admixtures thereof, or compositions are formulated in accordance with routine procedures as a pharmaceutical composition adapted for administration to human beings. Typically, compositions for intravenous administration, intra-arterial administration or administration within the kidney capsule, for example, are solutions in sterile isotonic aqueous buffer. Where necessary, the composition can also include a local anesthetic to ameliorate any pain at the site of the injection. Generally, the ingredients are supplied either separately or mixed together in unit dosage form, for example, as a cryopreserved concentrate in a hermetically sealed container such as an ampoule indicating the quantity of active agent. When the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Pharmaceutically acceptable carriers are determined in part by the particular composition being administered, as well as by the particular method used to administer the composition. Accordingly, there are a wide variety of suitable formulations of pharmaceutical compositions (see, e.g., Alfonso R Gennaro (ed), Remington: The Science and Practice of Pharmacy, formerly Remington's Pharmaceutical Sciences 20th ed., Lippincott, Williams & Wilkins, 2003, incorporated herein by reference in its entirety). The pharmaceutical compositions are generally formulated as sterile, substantially isotonic and in full compliance with all Good Manufacturing Practice (GMP) regulations of the U.S. Food and Drug Administration.

Cell Viability Agents

In one aspect, the bioactive cell formulation also includes a cell viability agent. In certain embodiments, the cell viability agent is selected from the group consisting of an antioxidant, an oxygen carrier, an immunomodulatory factor, a cell recruitment factor, a cell attachment factor, an anti-inflammatory agent, an angiogenic factor, a matrix metalloprotease, a wound healing factor, and products secreted from bioactive cells.

Antioxidants are characterized by the ability to inhibit oxidation of other molecules. Antioxidants include, without limitation, one or more of 6-hydroxy-2,5,7,8-tetramethylchroman-2-carboxylic acid (Trolox®), carotenoids, flavonoids, isoflavones, ubiquinone, glutathione, lipoic acid, superoxide dismutase, ascorbic acid, vitamin E, vitamin A, mixed carotenoids (e.g., beta carotene, alpha carotene, gamma carotene, lutein, lycopene, phytopene, phytofluene, and astaxanthin), selenium, Coenzyme Q10, indole-3-carbinol, proanthocyanidins, resveratrol, quercetin, catechins, salicylic acid, curcumin, bilirubin, oxalic acid, phytic acid, lipoic acid, vanilic acid, polyphenols, ferulic acid, theaflavins, and derivatives thereof. Those of ordinary skill in the art will appreciate other suitable antioxidants may be used in certain embodiments of the present disclosure.

Oxygen carriers are agents characterized by the ability to carry and release oxygen. They include, without limitation, perfluorocarbons and pharmaceuticals containing perfluorocarbons. Suitable perfluorocarbon-based oxygen carriers include, without limitation, perfluorooctyl bromide (C8F17Br); perfluorodichorotane (C8F16C12); perfluorodecyl bromide; perfluobron; perfluorodecalin; perfluorotripopylamine; perfluoromethylcyclopiperidine; Fluosol® (perfluorodecalin & perfluorotripopylamine); Perftoran® (perfluorodecalin & perfluoromethylcyclopiperidine); Oxygent® (perfluorodecyl bromide & perfluobron); Ocycyte™ (perfluoro (tert-butylcyclohexane)). Those of ordinary skill in the art will appreciate other suitable perfluorocarbon-based oxygen carriers may be used in certain embodiments of the present disclosure.

Immunomodulatory factors include, without limitation, osteopontin, FAS Ligand factors, interleukins, transforming growth factor beta, platelet derived growth factor, clusterin, transferrin, regulated upon action, normal T-cell expressed, secreted protein (RANTES), plasminogen activator inhibitor-1 (Pai-1), tumor necrosis factor alpha (TNF-alpha), interleukin 6 (IL-6), alpha-1 microglobulin, and beta-2-microglobulin. Those of ordinary skill in the art will appreciate other suitable immunomodulatory factors may be used in certain embodiments of the present disclosure.

Anti-inflammatory agents or immunosuppressant agents (described below) may also be part of the formulation. Those of ordinary skill in the art will appreciate other suitable antioxidants may be used in certain embodiments of the present disclosure.

Cell recruitment factors include, without limitation, monocyte chemotatic protein 1 (MCP-1), and CXCL-1. Those of ordinary skill in the art will appreciate other suitable cell recruitment factors may be used in certain embodiments of the present disclosure.

Cell attachment factors include, without limitation, fibronectin, procollagen, collagen, ICAM-1, connective tissue growth factor, laminins, proteoglycans, specific cell adhesion peptides such as RGD and YSIGR. Those of ordinary skill in the art will appreciate other suitable cell attachment factors may be used in certain embodiments of the present disclosure.

Angiogenic factors include, without limitation, vascular endothelial growth factor F (VEGF) and angiopoietin-2 (ANG-2). Those of ordinary skill in the art will appreciate other suitable angiogenic factors may be used in certain embodiments of the present disclosure.

Matrix metalloproteases include, without limitation, matrix metalloprotease 1 (MMP1), matrix metalloprotease 2 (MMP2), matrix metalloprotease 9 (MMP-9), and tissue inhibitor and matalloproteases-1 (TIMP-1).

Wound healing factors include, without limitation, keratinocyte growth factor 1 (KGF-1), tissue plasminogen activator (tPA), calbindin, clusterin, cystatin C, trefoil factor 3. Those of ordinary skill in the art will appreciate other suitable wound healing factors may be used in certain embodiments of the present disclosure.

Secreted products from bioactive cells described herein may also be added to the bioactive cell formulation as a cell viability agent.

Compositions sourced from body fluids, tissue or organs from human or animal sources, including, without limitation, human plasma, human platelet lysate, bovine fetal plasma or bovine pituitary extract, may also be added to the bioactive cell formulations as a cell viability agent.

Those of ordinary skill in the art will appreciate there are several suitable methods for depositing or otherwise combining cell populations with biomaterials to form a construct.

### 5. Methods of Use

In one aspect, the constructs and formulations of the present disclosure are suitable for use in the methods of use described herein. In certain embodiments, the formulations of the present disclosure may be administered for the treatment of disease. For example, bioactive cells may be administered to a native organ as part of a formulation described herein. In certain embodiments, the bioactive cells may be sourced from the native organ that is the subject of the administration or from a source that is not the target native organ.

In certain embodiments, the present disclosure provides methods for the treatment of a kidney disease, in a subject in need with the formulations containing bioactive renal cell populations as described herein. In certain embodiments, the therapeutic formulation contains a selected renal cell population or admixtures thereof. In embodiments, the formulations are suitable for administration to a subject in need of improved kidney function.

In another aspect, the effective treatment of a kidney disease in a subject by the methods of the present disclosure can be observed through various indicators of kidney function. In certain embodiments, the indicators of kidney function include, without limitation, serum albumin, albumin to globulin ratio (A/G ratio), serum phosphorous, serum sodium, kidney size (measurable by ultrasound), serum calcium, phosphorous:calcium ratio, serum potassium, proteinuria, urine creatinine, serum creatinine, blood nitrogen urea (BUN), cholesterol levels, triglyceride levels and glomerular filtration rate (GFR). Furthermore, several indicators of general health and well-being include, without limitation, weight gain or loss, survival, blood pressure (mean systemic blood pressure, diastolic blood pressure, or systolic blood pressure), and physical endurance performance.

In another aspect, an effective treatment with a bioactive renal cell formulation is evidenced by stabilization of one or more indicators of kidney function. The stabilization of kidney function is demonstrated by the observation of a change in an indicator in a subject treated by a method of the present disclosure as compared to the same indicator in a subject that has not been treated by a method of the present disclosure. Alternatively, the stabilization of kidney function may be demonstrated by the observation of a change in an indicator in a subject treated by a method of the present disclosure as compared to the same indicator in the same subject prior to treatment. The change in the first indicator may be an increase or a decrease in value. In certain embodiments, the treatment provided by the present disclosure may include stabilization of blood urea nitrogen (BUN) levels in a subject where the BUN levels observed in the subject are lower as compared to a subject with a similar disease state who has not been treated by the methods of the present disclosure. In one other embodiment, the treatment may include stabilization of serum creatinine levels in a subject where the serum creatinine levels observed in the subject are lower as compared to a subject with a similar disease state who has not been treated by the methods of the present disclosure. In certain embodiments, the stabilization of one or more of the above indicators of kidney function is the result of treatment with a selected renal cell formulation.

Those of ordinary skill in the art will appreciate that one or more additional indicators described herein or known in the art may be measured to determine the effective treatment of a kidney disease in the subject.

In another aspect, an effective treatment with a bioactive renal cell formulation is evidenced by improvement of one or more indicators of kidney function. In certain embodiments, the bioactive renal cell population provides an improved level of serum blood urea nitrogen (BUN). In certain embodiments, the bioactive renal cell population provides an improved retention of protein in the serum. In certain embodiments, the bioactive renal cell population provides improved levels of serum albumin as compared to the non-enriched cell population. In certain embodiments, the bioactive renal cell population provides improved A:G ratio as compared to the non-enriched cell population. In certain embodiments, the bioactive renal cell population provides improved levels of serum cholesterol and/or triglycerides. In certain embodiments, the bioactive renal cell population provides an improved level of Vitamin D. In certain embodiments, the bioactive renal cell population provides an improved phosphorus:calcium ratio as compared to a non-enriched cell population. In certain embodiments, the bioactive renal cell population provides an improved level of hemoglobin as compared to a non-enriched cell population. In a further embodiment, the bioactive renal cell population provides an improved level of serum creatinine as compared to a non-enriched cell population. In yet another embodiment, the bioactive renal cell population provides an improved level of hematocrit as compared to a non-enriched cell population. In certain embodiments, the improvement of one or more of the above indicators of kidney function is the result of treatment with a selected renal cell formulation.

In another aspect, the present disclosure provides formulations for use in methods for the regeneration of a native kidney in a subject in need thereof. In certain embodiments, the method includes the step of administering or implanting a bioactive cell population, admixture, or construct described herein to the subject. A regenerated native kidney may be characterized by a number of indicators including, without limitation, development of function or capacity in the native kidney, improvement of function or capacity in the native kidney, and the expression of certain markers in the native kidney. In certain embodiments, the developed or improved function or capacity may be observed based on the various indicators of kidney function described above. In certain embodiments, the regenerated kidney is characterized by differential expression of one or more stem cell markers. The stem cell marker may be one or more of the following: SRY (sex determining region Y)-box 2 (Sox2); Undifferentiated Embryonic Cell Transcription Factor (UTF1); Nodal Homolog from Mouse (NODAL); Prominin 1 (PROM1) or CD133 (CD133); CD24; and any combination thereof (see Ilagan et al. PCT/US2011/036347 incorporated herein by reference in its entirety), see also Genheimer et al., 2012. Molecular characterization of the regenerative response induced by intrarenal transplantation of selected renal cells in a rodent model of chronic kidney disease. Cells Tissue Organs 196: 374-384, incorporated by reference in its entirety. In certain embodiments, the expression of the stem cell marker(s) is up-regulated compared to a control.

In an aspect, provided herein is method of treating kidney disease in a subject, the method comprising injecting a formulation, composition, or cell population disclosed herein into the subject. In certain embodiments, the formulation, composition, for cell population is injected through a 18 to 30 gauge needle. In certain embodiments, the formulation, composition, for cell population is injected through a needle that is smaller than 20 gauge. In certain embodiments, the formulation, composition, for cell population is injected through a needle that is smaller than 21 gauge. In certain embodiments, the formulation, composition, for cell population is injected through a needle that is smaller than 22 gauge. In certain embodiments, the formulation, composition, for cell population is injected through a needle that is smaller than 23 gauge. In certain embodiments, the formulation, composition, for cell population is injected through a needle that is smaller than 24 gauge. In certain embodiments, the formulation, composition, for cell population is injected through a needle that is smaller than 25 gauge. In certain embodiments, the formulation, composition, for cell population is injected through a needle that is smaller than 26 gauge. In certain embodiments, the formulation, composition, for cell population is injected through a needle that is smaller than 27 gauge. In certain embodiments, the formulation, composition, for cell population is injected through a needle that is smaller than 28 gauge. In certain embodiments, the formulation, composition, for cell population is injected through a needle that is smaller than 29 gauge. In certain embodiments, the formulation, composition, for cell population is injected through a needle that is about 20 gauge. In certain embodiments, the formulation, composition, for cell population is injected through a needle that is about 21 gauge.

In certain embodiments, the formulation, composition, for cell population is injected through a needle that is about 22 gauge. In certain embodiments, the formulation, composition, for cell population is injected through a needle that is about 23 gauge. In certain embodiments, the formulation, composition, for cell population is injected through a needle that is about 24 gauge. In certain embodiments, the formulation, composition, for cell population is injected through a needle that is about 25 gauge. In certain embodiments, the formulation, composition, for cell population is injected through a needle that is about 26 gauge. In certain embodiments, the formulation, composition, for cell population is injected through a needle that is about 27 gauge. In certain embodiments, the formulation, composition, for cell population is injected through a needle that is about 28 gauge. In certain embodiments, the formulation, composition, for cell population is injected through a needle that is about 29 gauge.

In certain embodiments, the inter diameter of the needle is less than 0.84 mm. In certain embodiments, the inter diameter of the needle is less than 0.61 mm. In certain embodiments, the inter diameter of the needle is less than 0.51 mm. In certain embodiments, the inter diameter of the needle is less than 0.41 mm. In certain embodiments, the inter diameter of the needle is less than 0.33 mm. In certain embodiments, the inter diameter of the needle is less than 0.25 mm. In certain embodiments, the inter diameter of the needle is less than 0.20 mm. In certain embodiments, the inter diameter of the needle is less than 0.15 mm. In certain embodiments, the outer diameter of the needle is less than 1.27 mm. In certain embodiments, the outer diameter of the needle is less than 0.91 mm. In certain embodiments, the outer diameter of the needle is less than 0.81 mm. In certain embodiments, the outer diameter of the needle is less than 0.71 mm. In certain embodiments, the outer diameter of the needle is less than 0.64 mm. In certain embodiments, the outer diameter of the needle is less than 0.51 mm. In certain embodiments, the outer diameter of the needle is less than 0.41 mm. In certain embodiments, the outer diameter of the needle is less than 0.30 mm. In cetain embodiments, a needle has one of the sizes in the following table:

| | ID Size | | OD Size | |
|---|---|---|---|---|
| Gauge | in | mm | in | mm |
| 14 | 0.060 | 1.55 | 0.072 | 1.83 |
| 15 | 0.054 | 1.37 | 0.065 | 1.65 |
| 16 | 0.047 | 1.19 | n/a | n/a |
| 18 | 0.033 | 0.84 | 0.050 | 1.27 |
| 20 | 0.023 | 0.61 | 0.036 | 0.91 |
| 21 | 0.020 | 0.51 | 0.032 | 0.81 |
| 22 | 0.016 | 0.41 | 0.028 | 0.71 |
| 23 | 0.013 | 0.33 | 0.025 | 0.64 |
| 25 | 0.010 | 0.25 | 0.020 | 0.51 |
| 27 | 0.008 | 0.20 | 0.016 | 0.41 |
| 30 | 0.006 | 0.15 | 0.012 | 0.30 |
| 32 | 0.004 | 0.10 | 0.009 | 0.23 |

Secreted Products

In certain embodiments, the effect may be provided by the cells themselves and/or by products secreted from the cells. The regenerative effect may be characterized by one or more of the following: a reduction in epithelial-mesenchymal transition (which may be via attenuation of TGF-β signaling); a reduction in renal fibrosis; a reduction in renal inflammation; differential expression of a stem cell marker in the native kidney; migration of implanted cells and/or native cells to a site of renal injury, e.g., tubular injury, engraftment of implanted cells at a site of renal injury, e.g., tubular injury; stabilization of one or more indicators of kidney function (as described herein); de novo formation of S-shaped bodies/comma-shaped bodies associated with nephrogenesis, de novo formation of renal tubules or nephrons, restoration of erythroid homeostasis (as described herein); and any combination thereof (see also Basu et al., 2011. Functional evaluation of primary renal cell/biomaterial neo-kidney augment prototypes for renal tissue engineering. Cell Transplantation 20: 1771-90; Bruce et al., 2015. Selected renal cells modulate disease progression in rodent models of chronic kidney disease via NF-κB and TGF-β1 pathways. Regenerative Medicine 10: 815-839, the entire content of each of which is incorporated herein by reference).

As an alternative to a tissue biopsy, a regenerative outcome in the subject receiving treatment can be assessed from examination of a bodily fluid, e.g., urine. It has been discovered that microvesicles obtained from subject-derived urine sources contain certain components including, without limitation, specific proteins and miRNAs that are ultimately derived from the renal cell populations impacted by treatment with the cell populations of the present disclosure. These components may include, without limitation, factors involved in stem cell replication and differentiation, apoptosis, inflammation and immuno-modulation, fibrosis, epithelial-mesenchymal transition, TGF-β signaling and PAI-1 signaling A temporal analysis of microvesicle-associated miRNA/protein expression patterns allows for continuous monitoring of regenerative outcomes within the kidney of subjects receiving the cell populations, admixtures, or constructs of the present disclosure.

In certain embodiments, the present disclosure provides methods of assessing whether a kidney disease (KD) patient is responsive to treatment with a therapeutic formulation. The method may include the step of determining or detecting the amount of vesicles or their luminal contents in a test sample obtained from a KD patient treated with the therapeutic, as compared to or relative to the amount of vesicles in a control sample derived from the same patient prior to treatment with the therapeutic, wherein a higher or lower amount of vesicles or their luminal contents in the test sample as compared to the amount of vesicles or their luminal contents in the control sample is indicative of the treated patient's responsiveness to treatment with the therapeutic.

These kidney-derived vesicles and/or the luminal contents of kidney derived vesicles may also be shed into the urine of a subject and may be analyzed for biomarkers indicative of regenerative outcome or treatment efficacy. The non-invasive prognostic methods may include the step of obtaining a urine sample from the subject before and/or after administration or implantation of a cell population, admixture, or construct described herein. Vesicles and other secreted products may be isolated from the urine samples using standard techniques including without limitation, centrifugation to remove unwanted debris (Zhou et al. 2008. Kidney Int. 74(5):613-621; Skog et al. U.S. Published Patent Application No. 20110053157, each of which is incorporated herein by reference in its entirety) precipitation to separate exosomes from urine, polymerase chain reaction and nucleic acid sequencing to identify specific nucleic acids and mass spectroscopy and/or 2D gel electrophoresis to identify specific proteins associated with regenerative outcomes.

The foregoing written description is considered to be sufficient to enable one skilled in the art to practice the invention. It should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

All patents, patent applications, and literature references cited in the present specification are hereby incorporated by reference in their entirety.

### Examples

### Example 1: NKA Formulation Components

1. Cellular Components and Materials

SRC constitute the biologically active component of NKA. SRC are composed primarily of renal tubular epithelial cells that are well known for their regenerative potential. Other parenchymal (vascular), mesenchymal, endothelial and stromal (collecting duct) cells may be present in the autologous SRC population.

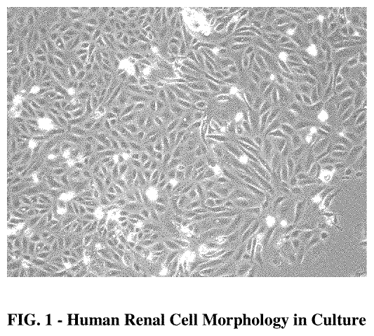

SRC are prepared from renal cortical tissue obtained using a standard-of-clinical-care kidney biopsy procedure to collect cores of kidney tissue. Renal cells are isolated from the kidney tissue by enzymatic digestion and expanded using standard cell culture techniques. Cells are assessed to verify renal cell morphology by visual observation of cultures under the microscope. Cultures characteristically demonstrate a tight pavement or cobblestone appearance, due to the cells clustering together (FIG. 1). SRC are obtained by separation of the isolated and expanded cells across a density boundary or density interface or single step discontinuous density gradient.

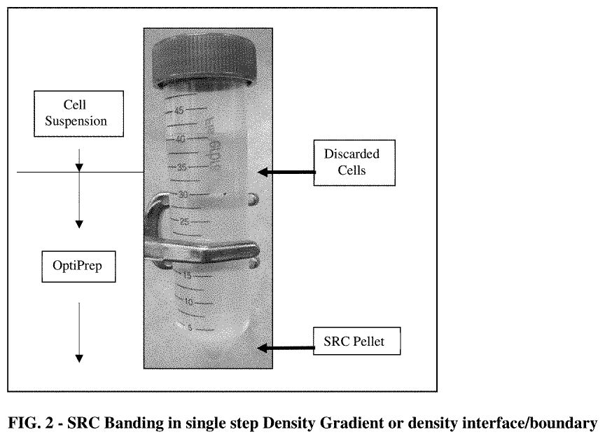

Centrifugation across a density boundary or interface is used to separate harvested renal cell populations based on cell buoyant density. Renal cell suspensions are separated over a solution of OptiPrep (7% iodixanol; 60% (w/v) in OptiMEM) medium. The cellular fraction exhibiting buoyant density greater than approximately 1.0419 g/mL is collected after centrifugation as a distinct pellet (FIG. 2). Cells maintaining a buoyant density of less than 1.0419 g/mL are excluded and discarded.

The SRC pellet is re-suspended in DPBS. The carry-over of residual OptiPrep, FBS, culture medium and ancillary materials in the final product is minimized by washing steps.

2. Biomaterial Components and Ancillary Materials

The following biomaterial components and ancillary materials are used for formulation of SRC into NKA:
1. Porcine gelatin—used to make the thermally responsive hydrogel.
2. Dulbecco's phosphate-buffered saline (DPBS)—used to dissolve the porcine gelatin. The buffer may be replaced or mixed with human plasma or human platelet lysate.

Biomaterial Preparation

The biomaterial is a Gelatin Solution composed of porcine gelatin in DPBS. Gelatin is dissolved in DPBS or human plasma/human platelet lysate or a mixture of both to a specified concentration to form a Gelatin Solution of a thermally responsive hydrogel. The Gelatin Solution is filter sterilized through a 0.1 μm filter and stored refrigerated or frozen in single use aliquots ready for formulation.

The key property of the biomaterial is that it is a thermally responsive hydrogel such that it can gel and liquefy at different temperatures. Gelatin Solution used in NKA formulation is liquid at and above room temperature (22-28° C.) and gels when cooled to refrigerated temperatures (2-8° C.).

Gelatin Solution Concentration

Gelatin concentration in the range of 0.5-1.0% was evaluated for gelation properties—ability to form a gel at refrigerated temperature (no flow when inverted) and to become fluid at room temperature (free flowing when inverted). Table 4 shows gelation properties of different concentration of Gelatin Solution.

TABLE 4
Gelation Properties of Gelatin Solution at Different Concentrations

| | Gel | |
|---|---|---|
| Gelatin | (Refrigerated | Flow |
| Concentration | Temperature) | (Room Temperature) |
| 0.50% | +/− | + |
| 0.63% | + | + |
| 0.75% | + | + |
| 0.88% | + | + |
| 1.00% | + | + |

Since NKA formulated with gelatin solution of 0.63% and above were able to consistently meet the acceptance criteria, a range of 0.88±0.12% was selected for gelatin concentration for NKA formulation. It is noted, however, that formulations comprising gelatin in the concentration range of about 0.63% to about 1% are also suitable.

3. NKA Formulation

SRC are formulated into NKA with Gelatin Solution, a gelatin-based thermally responsive hydrogel. The gelatin-based thermally responsive hydrogel provides improved stability of the cells thus extending product shelf life, stability during transport and delivery of SRC into the kidney cortex for clinical utility. Formulation development assessed composition, concentration and stability of Gelatin Solution.

Washed SRC are counted using Trypan Blue dye exclusion. Gelatin Solution is removed from cold storage and liquefied by warming to 26-30° C. A volume of SRC suspension containing the required number of cells is centrifuged and re-suspended in liquefied Gelatin Solution for a final wash step. This suspension is centrifuged and the SRC pellet is re-suspended in sufficient Gelatin Solution to achieve a resultant SRC concentration of 100×10⁶ cells/mL in the formulated NKA.

NKA Filling and Gelation

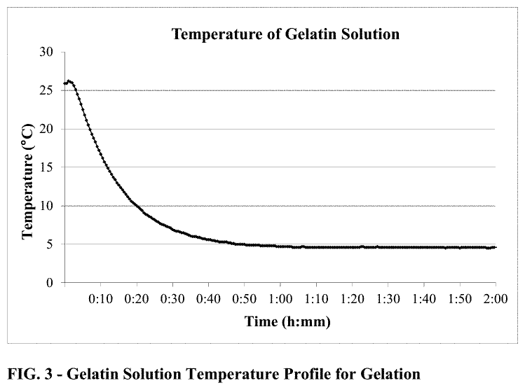

NKA product is aseptically filled into a syringe. Dynamic air sampling is performed for the duration of the filling process, including viable and non-viable sampling. The NKA package is rotated for a minimum of 2 hours to keep the cells in suspension while cooling to 2-8° C. to form the final gelled NKA. Rapid cooling is required for gelation to take place so that cells do not settle in the Gelatin Solution. The temperature of the Gelatin Solution in a syringe was monitored as it was placed into refrigerated conditions. Rapid temperature drop is observed as shown in FIG. 3. After 1 hour, the temperature typically drops to within 0.3° C. of the final temperature 4.4° C.

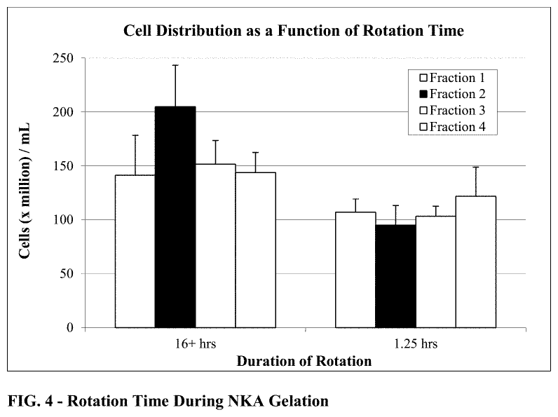

Cooling of the Gelatin Solution starts the gelation process but a finite amount to time is required for the formed gel to stabilize such that the SRC will remain suspended in the gel on storage. Syringes containing formulated NKA were rotated either overnight or for 1.25 hours and then held upright overnight. Subsequently, the contents were removed and cell concentration was measured in four different segments of the product. Analysis indicates that there is no difference among the four segments, thus no measurable cell settling occurs once NKA has rotated at cold temperature for a minimum of 1.25 hours (FIG. 4).

### Example 2: Characterization of NKA and Components

NKA and its components, SRC and Biomaterial, have been characterized using analytical techniques described in this section.

Characterization of SRC

SRC have been characterized for release testing purposes and in extended culture for qualification purposes. In addition, SRC have been tested for other characteristics that may be used for informational and developmental purposes and may be helpful in establishing potency assays in the future.

SRC Characteristics

Renal cell isolation and expansion provides a mixture of renal cell types including renal tubular epithelial cells and stromal cells. SRC are obtained by single step discontinuous density gradient separation of the expanded renal cells or by centrifugation across a density boundary/densitry interface. The primary cell type in the density separated SRC population is of epithelial phenotype. A multi-pronged approach was taken to establish the characteristics of SRC obtained from expanded renal cells. Cell morphology, growth kinetics and cell viability are monitored during the renal cell expansion process. SRC buoyant density is established by use of centrifugation across a density interface. Cell count and viability are measured by Trypan Blue dye exclusion. SRC phenotype is characterized by flow cytometry. The presence of viable cells and SRC function is demonstrated by metabolism of PrestoBlue and production of VEGF and KIM-1.

SRC used in the manufacture of NKA for clinical studies will be tested for the following key characteristics:
SRC Count and Viability
SRC Phenotype
SRC Function

SRC Count and Viability

Cell count and viability are measured by Trypan Blue dye exclusion.

SRC Phenotype

Cell phenotype is monitored by expression analysis of renal cell markers using flow cytometry. Phenotypic analysis of cells is based on the use of antigenic markers specific for the cell type being analyzed. Flow cytometric analysis provides a quantitative measure of cells in the sample population which express the antigenic marker being analyzed.

A variety of markers have been reported in the literature as being useful for phenotypic characterization of renal cells: (i) cytokeratins; (ii) transport membrane proteins (aquaporins and cubilin); (iii) cell binding molecules (adherins, lectins, and others); and (iv) metabolic enzymes (glutathione). Since the majority of cells found in cultures derived from whole kidney digests are epithelial and endothelial cells, the markers examined focus on the expression of proteins specific for these two groups.

Cytokeratins are a family of intermediate filament proteins expressed by many types of epithelial cells to varying degrees. The subset of cytokeratins expressed by an epithelial cell depends upon the type of epithelium. For example, cytokeratins 7, 8, 18 and 19 are all expressed by normal simple epithelia of the kidney and remaining urogenital tract as well as the digestive and respiratory tracts. These cytokeratins in combination are responsible for the structural integrity of epithelial cells. This combination represents both the acidic (type I) and basic (type II) keratin families and is found abundantly expressed in renal cells (Oosterwijk et al. (1990) Expression of intermediate-sized filaments in developing and adult human kidney and in renal cell carcinoma. J Histochem Cytochem, 38(3), 385-392).

Aquaporins are transport membrane proteins which allow the passage of water into and out of the cell, while preventing the passage of ions and other solutes. There are thirteen aquaporins described in the literature, with six of these being found in the kidney (Nielsen et al. (2002) Aquaporins in the kidney: from molecules to medicine. Physiol Rev, 82(1), 205-244). Aquaporin2, by exerting tight control in regulating water flow, is responsible for the plasma membranes of renal collecting duct epithelial cells having a high permeability to water, thus permitting water to flow in the direction of an osmotic gradient (Bedford et al. (2003) Aquaporin expression in normal human kidney and in renal disease. J Am Soc Nephrol, 14(10), 2581-2587; Takata et al. (2008) Localization and trafficking of aquaporin 2 in the kidney. Histochem Cell Biol, 130(2), 197-209; Tamma et al. (2007) Hypotonicity induces aquaporin-2 internalization and cytosol-to-membrane translocation of ICln in renal cells. Endocrinology, 148(3), 1118-1130). Aquaporin1 is characteristic of the proximal tubules (Baer et al. (2006) Differentiation status of human renal proximal and distal tubular epithelial cells in vitro: Differential expression of characteristic markers. Cells Tissues Organs, 184(1), 16-22; Nielsen et al. (2002) Aquaporins in the kidney: from molecules to medicine. Physiol Rev, 82(1), 205-244).

Cubilin is a transport membrane receptor protein. When it co-localizes with the protein megalin, together they promote the internalization of cubilin-bound ligands such as albumin. Cubilin is located within the epithelium of the intestine and the kidney (Christensen & Birn (2001) Megalin and cubilin: synergistic endocytic receptors in renal proximal tubule. Am J Physiol Renal Physiol, 280(4), F562-573).

CXCR4 is a transport membrane protein which serves as a chemokine receptor for SDF1. Upon ligand binding, intracellular calcium levels increase and MAPK1/MAPK3 activation is increased. CXCR4 is constitutively expressed in the kidney and plays an important role in kidney development and tubulogenesis (Ueland et al. (2009). A novel role for the chemokine receptor Cxcr4 in kidney morphogenesis: an in vitro study. Dev Dyn, 238(5), 1083-1091). Additionally, CXCR4 is the receptor for ligand binding of SDF1. The SDF1/CXCR4 axis plays a crucial role in the migration and homing of endothelial progenitor cells and mesenchymal stem cells to sites of injury (Stem-cell approaches for kidney repair: choosing the right cells. (Sagrinati et al. Trends Mol Med. 2008; 14(7):277-85).

Cadherins are calcium-dependent cell adhesion proteins. They are classified into four groups, with the E-cadherins being found in epithelial tissue, and are involved in regulating mobility and proliferation. E-cadherin is a transmembrane glycoprotein which has been found to be localized in the adherins junctions of epithelial cells which make up the distal tubules in the kidney (Prozialeck et al. (2004) Differential expression of E-cadherin, N-cadherin and beta-catenin in proximal and distal segments of the rat nephron. BMC Physiol, 4, 10; Shen et al. (2005) Kidney-specific cadherin, a specific marker for the distal portion of the nephron and related renal neoplasms. Mod Pathol, 18(7), 933-940).

DBA (Dolichos biflorus agglutinin) is an α-N-acetylgalactosamine-binding lectin (cell binding protein) carried on the surface of renal collecting duct structures, and is regarded and used as a general marker of developing renal collecting ducts and distal tubules (Michael et al. (2007) The lectin Dolichos biflorus agglutinin is a sensitive indicator of branching morphogenetic activity in the developing mouse metanephric collecting duct system. J Anat 210(1), 89-97; Lazzeri et al. (2007) Regenerative potential of embryonic renal multipotent progenitors in acute renal failure. J Am Soc Nephrol 18 (12), 3128-3138).

CD31 (also known as platelet endothelial cell adhesion molecule, PECAM-1) is a cell adhesion protein which is expressed by select populations of immune cells as well as endothelial cells. In endothelial cells, this protein is concentrated at the cell borders (DeLisser et al. (1997) Involvement of endothelial PECAM-1/CD31 in angiogenesis. Am J Pathol, 151(3), 671-677). CD146 is involved in cell adhesion and cohesion of endothelial cells at intercellular junctions associated with the actin cytoskeleton. Strongly expressed by blood vessel endothelium and smooth muscle, CD146 is currently used as a marker for endothelial cell lineage (Malyszko et al. (2004) Adiponectin is related to CD146, a novel marker of endothelial cell activation/injury in chronic renal failure and peritoneally dialyzed patients. J Clin Endocrinol Metab, 89(9), 4620-4627), and is the canine equivalent of CD31.

Gamma-glutamyl transpeptidase (GGT) is a metabolic enzyme that catalyzes the transfer of the gamma-glutamyl moiety of glutathione to an acceptor that may be an amino acid, a peptide, or water, to form glutamate. This enzyme also plays a role in the synthesis and degradation of glutathione and the transfer of amino acids across the cell membrane. GGT is present in the cell membranes of many tissues, including the proximal tubule cells of kidneys (Horiuchi et al. (1978) Gamma-glutamyl transpeptidase: sidedness of its active site on renal brush-border membrane. Eur J Biochem, 87(3), 429-437; Pretlow et al. (1987). Enzymatic histochemistry of mouse kidney in plastic. J Histochem Cytochem, 35(4), 483-487; Welbourne & Matthews (1999) Glutamate transport and renal function. Am J Physiol, 277(4 Pt 2), F501-505). Table 5 provides a list of the specific types of renal cells expressing these markers as detected by flow cytometry.

TABLE 5
Phenotypic Markers for SRC Characterization

| Antigenic marker | Reactivity |
|---|---|
| CK8/18/19 | Epithelial cells, proximal and distal tubules |
| CK8 | Epithelial cells, proximal tubules |
| CK18 | Epithelial cells, proximal tubules |
| CK19 | Epithelial cells, collecting ducts, distal tubules |
| CK7 | Epithelial cells, collecting ducts, distal tubules |
| CXCR4 | Epithelial cells, distal and proximal tubules |
| E-cadherin | Epithelial cells, distal tubules |
| Cubilin | Epithelial cells, proximal tubules |
| Aquaporin1 | Epithelial cells, proximal tubules, descending |
| | thin limb |
| GGT1 | Fetal and adult kidney cells, proximal tubules |
| Aquaporin2 | Renal collecting duct cells, distal tubules |
| DBA | Renal collecting duct cells, distal tubules |
| CD31 | Endothelial cells of the kidney (rat) |
| CD146 | Endothelial cells of the kidney (canine, human) |

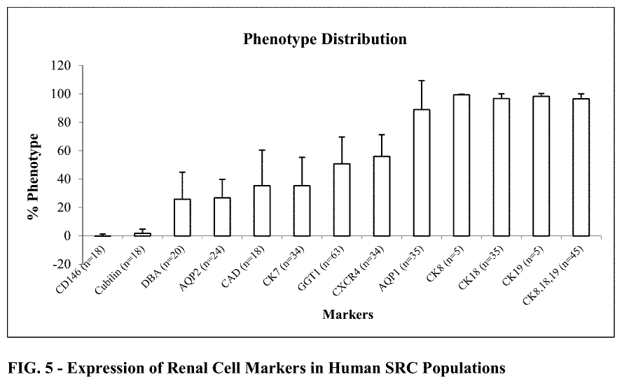

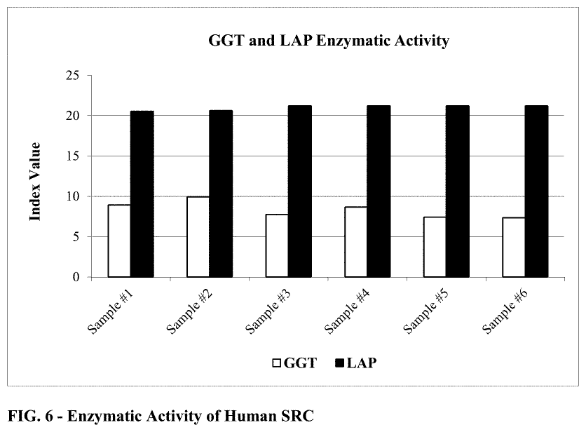

FIG. 5 shows quantified expression of these markers in SRC populations plotted as percentage values of each phenotype in the population. CK8/18/19 are the most consistently expressed renal cell proteins detected across species. GGT1 and Aquaporin-1 (AQP1) are expressed consistently but at varying levels. DBA, Aquaporin2 (AQP2), E-cadherin (CAD), CK7, and CXCR4 are also observed at modest levels though with more variability, and CD31/146 and Cubilin were lowest in expression. Based on the published data (Kelley et al. (2012) A Population of Selected Renal Cells Augments Renal Function and Extends Survival in the ZSF1 model of Progressive Diabetic Nephropathy. Cell Transplant 22(6), 1023-1039; Kelley et al. (2010a) A tubular cell-enriched subpopulation of primary renal cells improves survival and augments kidney function in a rodent model of chronic kidney disease. Am J Physiol Renal Physiol. 299(5), F1026-1039) and our unpublished work (FIG. 5), we have selected CK18 and GGT1 as the markers that will be utilized in routine phenotypic analysis of SRC during the manufacture of NKA. AQP2 expression is also a useful marker for phenotypic analysis but expression is variable and therefore AQP2 expression will be monitored for informational purposes. Table 6 provides the selected markers, range and mean percentage values of phenotypic expression in SRC and the rationale for their selection.

TABLE 6
Marker Selected for Phenotypic Analysis of SRC

| Phenotypic | Expression | | | Expression |
|---|---|---|---|---|
| Marker | Range | Average | Rationale | Level |
| CK18 | 81.1 to 99.7% | 96.7% | Epithelial marker | High |
| | (n = 87) | | | |
| GGT1 | 4.5 to 81.2% | 50.7% | Functional Tubular | Moderate |
| | (n = 63) | | marker | |

* Collecting duct epithelial cells are expected to be low in SRC based on their buoyant density.

Cell Function

SRC actively secrete proteins that can be detected through analysis of conditioned medium. Cell function is assessed by the ability of cells to metabolize PrestoBlue and secrete VEGF (Vascular Endothelial Growth Factor) and KIM-1 (Kidney Injury Molecule-1).

Viable functioning cells can be monitored in NKA by their ability to metabolize PrestoBlue. PrestoBlue Cell Viability Reagent is a modified resazurin-based assay reagent that is a cell permeable, non-fluorescent blue dye. Upon entry into cells which are sufficiently viable to proliferate, the dye is reduced, via natural cell processes involving dehydrogenase enzymes, to a bright red fluorophore that can be measured by fluorescence or absorbance.

Biomolecules VEGF and KIM-1 represent a selection of molecules from those proposed as sensitive and specific analytical nonclinical biomarkers of kidney injury and function (Sistare et al. (2010) Towards consensus practices to qualify safety biomarkers for use in early drug development. Nat Biotechnol, 28(5), 446-454; Warnock & Peck (2010) A roadmap for biomarker qualification. Nat Biotechnol, 28(5), 444-445). In vivo, both of these markers are indicative of tubular function, injury and/or repair and in vitro are recognized features of tubular epithelial cell cultures. KIM-1 is an extracellular protein anchored in the membrane of renal proximal tubule cells that serves to recognize and phagocytose apoptotic cells which are shed during injury and cell turnover. VEGF, constitutively expressed by kidney cells, is a pivotal angiogenic and pro-survival factor that promotes cell division, migration, endothelial cell survival and vascular sprouting. SRC have been characterized as constitutively expressing VEGF mRNA (Table 8) and actively produce the protein (Table 7). These proteins may be detected in culture medium exposed to renal cells and SRC. Table 7 presents VEGF and KIM-1 quantities present in conditioned medium from renal cells and SRC cultures. Renal cells were cultured to near confluence. Conditioned medium from overnight exposure to the renal cell cultures and SRC was tested for VEGF and KIM-1.

TABLE 7
Production of VEGF and KIM-1 by Human Renal Cells and SRC

| | VEGF | | KIM-1 | |
|---|---|---|---|---|
| Conditioned | | ng/million | | ng/million |
| Medium | ng/mL | cells | ng/mL | cells |
| Renal Cell | 0.50 to 2.42 | 2.98 to 14.6 | 0.20 to 3.41 | 1.14 to 15.2 |
| Culture | | | | |
| (n = 15) | | | | |
| SRC | 0.80 to 3.85 | 4.83 to 23.07 | 0.32 to 2.10 | 1.93 to 12.59 |
| (n = 14) | | | | |

Elucidation of Other SRC Characteristics

SRC have been further characterized by gene expression profiling, and measurement of enzymatic activity of the cells.

Gene Expression Profile

The gene expression profile of SRC isolated from human renal cell cultures were investigated by quantitative real-time polymerase chain reaction (qPCR), including aquaporin2, E-cadherin, cubulin, VEGF and CD31 that were also tested for protein production. Genotypic markers in Table 14 are representative of cell populations that might be expected to be found in the renal cell cultures. NCAD, Cubilin and CYP2R1 are markers of tubular epithelial cells, AQP2 and ECAD are markers of collecting duct and distal tubules. Podocin and Nephrin are markers of podocytes. VEGF and CD31 are endothelial markers. VEGF and EPO are oxygen responsive genes with related mRNA present in a variety of different tissue and cell types.

Gene probes used were obtained from TaqMan. Passage 2 human renal cells were harvested at 70-90% confluence. RNA was purified from the cells using Qiagen's RNeasy Plus Mini Kit following the protocol for Purification of Total RNA from Animal Cells. cDNA was generated from a volume of RNA equal to 1.4 μg using Invitrogen's SuperScript® VILO™ cDNA Synthesis Kit following the manufacturer's instructions. Averaged qPCR data for SRC populations (n=3) is shown in Table 8 relative to unfractionated renal cells.

The results suggest that a population of tubular epithelial cells is present as evidenced by relatively higher level of expression of NCAD, Cubilin and CYP2R1. Distal Collecting Duct Tubule and Distal Tubule markers AQP2 and ECAD are relatively low and CD31, an endothelial marker is even lower (Table 8).

TABLE 8
Gene Expression Analysis of Human SRC

| | Gene | | Human |
|---|---|---|---|
| Gene Name | Designation | Marker | Std Error |
| Aquaporin2 | AQP2 | Distal Tubule, | 0.201 |
| | | Collecting Duct | |
| E-cadherin/Cadherin 1, | ECAD/CDH1 | Distal Tubule | 0.191 |
| Type 1 | | | |
| Neuronal Cadherin/ | NCAD/CDH2 | Proximal Tubule | 0.208 |
| Cadherin 2, Type 1 | | | |
| Cubilin | CUBN | Tubular | 1.036 |
| Nephrin | NPHS1 | Podocyte | 0.422 |
| Podocin | NPHS2 | Podocyte | 0.000 |
| Elythropoietin | EPO | Cortical Fibroblast | 0.426 |
| Vitamin D 24- | CYP2R1 | Tubular | 0.028 |
| Hydroxylase | | | |
| Vascular Endothelial | VEGFA | Endothelial | 0.121 |
| Growth Factor A | | | |
| Platelet/Endothelial Cell | PECAM1/CD31 | Endothelial | 0.005 |
| Adhesion Molecule/CD31 | | | |

Phenotypic and functional markers have been chosen based upon early genotypic evaluation. VEGF gene expression levels are high and aquaporin2 gene expression levels are low which is consistent with the protein analysis data (Table 6 and Table 7).

SRC Enzymatic Activity

Cell function of SRC, pre-formulation, can also be evaluated by measuring the activity of two specific enzymes; GGT (γ-glutamyl transpeptidase) and LAP (leucine aminopeptidase) (Chung et al. (1982) Characterization of primary rabbit kidney cultures that express proximal tubule functions in a hormonally defined medium. J Cell Biol, 95(1), 118-126), found in kidney proximal tubules. Methods to measure the activity of these enzymes in cells utilize an enzyme-specific substrate in solution that, when added to cells expressing active enzyme, are cleaved, releasing a chromogenic product (Nachlas et al. (1960) Improvement in the histochemical localization of leucine aminopeptidase with a new substrate, L-leucyl-4-methoxy-2-naphthylamide. J Biophys Biochem Cytol, 7( ), 261-264; Tate & Meister (1974) Stimulation of the hydrolytic activity and decrease of the transpeptidase activity of gamma-glutamyl transpeptidase by maleate; identity of a rat kidney maleate-stimulated glutaminase and gamma-glutamyl transpeptidase. Proc Natl Acad Sci USA, 71(9), 3329-3333). The absorbance of the cell-exposed solution is measured and is relative to the amount of cleavage product resulting from active enzyme. The substrate utilized for GGT is L-glutamic acid γ-p-nitroanalide hydrochloride and for LAP is L-leucinep-nitroanalide. FIG. 6 shows LAP and GGT activity in 6 SRC samples produced from human donors. LAP and GGT assays are performed for information only. The assays require a long cell culture duration and therefore cannot be performed for product release.

Summary of SRC Characterization:
Cell morphology is monitored during cell expansion by comparison of culture observations with images in the Image Library.
Cell growth kinetics are monitored at each cell passage. Cell growth is expected to be variable from patient to patient.
SRC counts and viability are monitored by Trypan Blue dye exclusion and metabolism of PrestoBlue.
SRC are characterized by phenotypic expression of CK18, GGT1. AQP2 expression will be monitored for informational purposes.
Metabolism of PrestoBlue and production of VEGF and KIM-1 are used as markers for the presence of viable and functional SRC.
SRC function can be further elucidated with gene expression profiling and measurement of enzymatic activity with LAP and GGT.

Characterization of Biomaterials

The Biomaterial used in NKA (Gelatin Solution) is characterized via two key parameters:

Concentration—Concentration of Gelatin Solution is measured by absorbance at 280 nm using a spectrophotometer. The gelatin concentration is determined from a calibration curve of absorbance versus concentration.

Inversion Test—The inversion test provides a visual assessment of the ability of the Gelatin Solution to form and maintain a gel at a temperature of 2-8° C. and for the gel to liquefy (flow) at room temperature.

Elucidation of Other Biomaterial Characteristics

Biomaterials used in NKA can be further characterized for the rheological properties and viscosity.

Rheological Properties

Rheological properties of the Biomaterial can be measured first at 4° C., then at 25° C. through the use of a Couette Cell style rheometer. The sample is equilibrated for at least 30 minutes at each temperature. An acceptable storage modulus (G′>10) at the lower temperature reflects the ability of the solution to form and maintain a gel at NKA shipping and transport temperature of 2-8° C. An acceptable loss modulus (G″<10) at the higher temperature reflects the ability of the gel to liquefy at room temperature as required for delivery and implantation of NKA.

Viscosity Viscosity of the Biomaterial is measured using a cone and plate viscometer at 37° C. and a shear rate of 200-300 s⁻¹. Solutions with viscosities in range of 1.05-1.35 cP can be efficiently delivered through 18-27 gauge needles.

Characterization of NKA

The NKA is composed of autologous, SRC formulated in a Biomaterial (gelatin-based hydrogel). Formulation of SRC in a gelatin-based hydrogel biomaterial provides enhanced stability of the cells thus extending product shelf life, improved stability of NKA during transport and delivery of SRC into the kidney cortex for clinical utility.

NKA is characterized for presence of viable cells, SRC phenotype and cell function by metabolism of PrestoBlue, phenotypic expression of CK18, GGT1 and AQP2 and production of VEGF and KIM-1. Details are provided in the Characterization of SRC section above.

We conducted experiments to demonstrate that NKA produced with SRC obtained from human kidney donors and formulated with gelatin maintains uniform distribution of cells, without aggregation, within the syringe during storage and transportation thereby assuring improved stability of cells in the final NKA product post release and at injection. Results of SRC distribution and aggregation in NKA are provided in sections below. Details on stability of NKA on cold storage are provided below.

SRC Distribution in NKA

SRC distribution in NKA was established with qualitative observation of cell settling, imaging of live/dead viability using confocal microscopy and measurement of live cell distribution using Trypan blue dye exclusion.

Qualitative Observation of Cell Settling

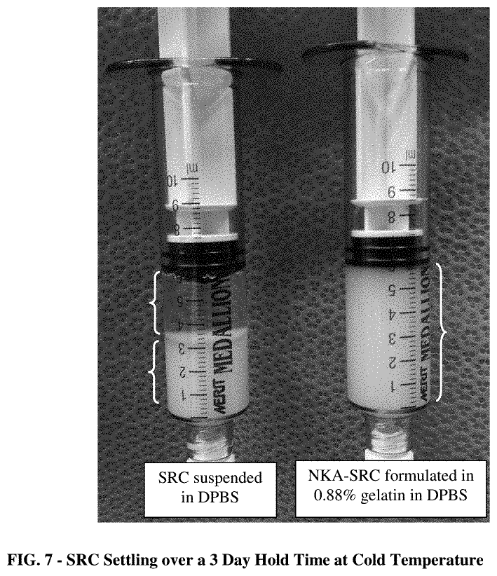

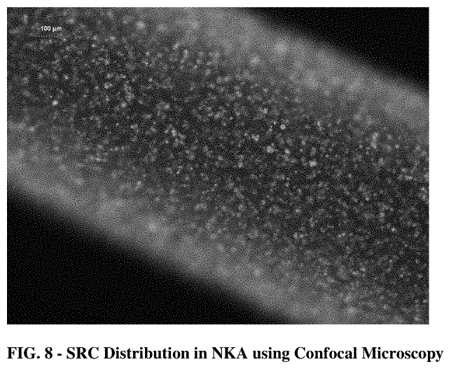

SRC in formulated NKA was visually observed for settling and compared to SRC suspended in DPBS only. SRC suspended in DPBS settle out of suspension during the hold period. NKA formulation of SRC with 0.88% gelatin in DPBS was able to keep cells from settling in the syringe over the 3 days of storage at cold temperatures (FIG. 7).

Imaging of Live/Dead Viability Using Confocal Microscopy

SRC distribution within the formulated NKA was imaged using confocal microscopy (BD Pathway 855). NKA (SRC formulated in gelatin) was expelled onto a glass chamber slide and stained with a fluorescent Live (green)/Dead (red) dye. FIG. 8 shows a representative image of viable SRC (green) distributed within the gelatin.

SRC Distribution Across NKA Syringe

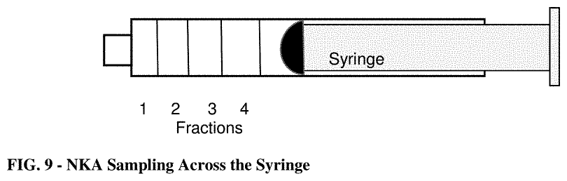

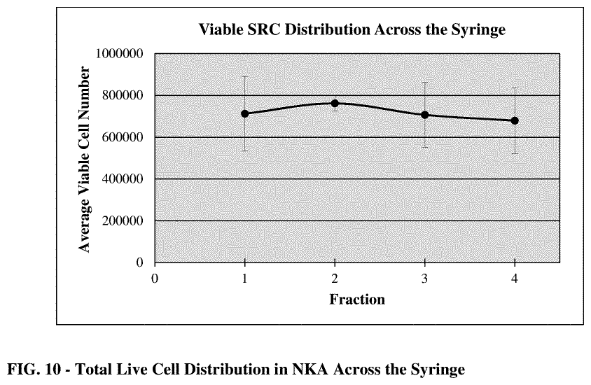

SRC distribution across formulated NKA syringes was measured using Trypan Blue staining. NKA was prepared in syringes using standard procedures. After holding for 3 days at cold temperatures and warmed to room temperature, NKA was expelled in four fractions from the syringes as shown in FIG. 9. Counts were performed for each fraction and the total live cell distribution and average viability determined.

Measurement of SRC Distribution in Syringe

SRC were counted in the expelled fraction using Trypan Blue dye exclusion. FIG. 10 shows total viable cell count at selected fractions illustrating distribution pattern along barrel of syringe at time of deposition. SRC are uniformly distributed across the syringe.

SRC Aggregation in NKA

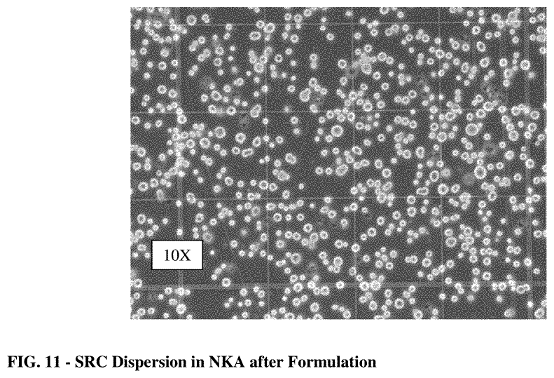

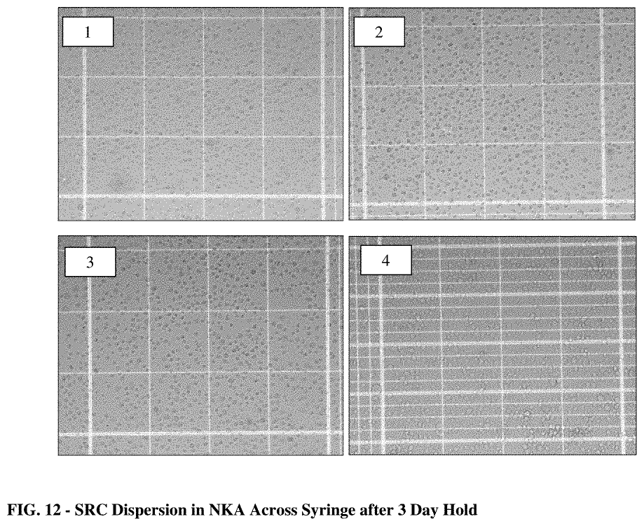

SRC aggregation in NKA was assessed using Leica LAS image software under phase contrast microscopy. Cell aggregation was assessed at formulation and also after a 3 day hold period at cold temperatures. FIG. 11 shows a Leica image of SRC immediately post formulation (10×). No aggregation of cells is observed in NKA formulation of SRC suspended in 0.88% gelatin. FIG. 12 shows phase contrast images (10×) of samples taken from NKA (fractions 1-4). No cell aggregation is observed across the syringe after the 3 day hold period.

Summary of NKA Characterization:
Gelatin formulation of SRC enables cells to remain suspended and distributed in NKA during storage and transport of NKA. Gelatin formulation also ensures uniform delivery of NKA during injection.
SRC suspended in DPBS only settle out during storage at cold temperature for 3 days.
SRC do not aggregate in NKA post formulation or upon storage during its product shelf life of 3 days.

### Example 3: Stability Testing

Gelatin Solution Stability

Prepared Gelatin Solution is stored in the refrigerator (2-8° C.) or freezer (below −20° C.). The stability of gelatin solution used for NKA formulation was evaluated after holding the material at cold temperatures (2-8° C.) for up to 8 weeks or frozen (below −20° C.) for up to 24 weeks.

After filter sterilization, Gelatin Solution was aliquoted into 15 mL tubes and stored, either in a refrigerator (2-8° C.) or freezer (below −20° C.). At the time of evaluation, one tube of Gelatin Solution was removed from the cold storage and placed in a 26-30° C. water bath. After 2 hours in the water bath, if the Gelatin Solution was observed to “flow” when the tube was inverted, the solution was deemed acceptable for ability to liquefy. The tube was returned to 2-8° C. cold storage and observed the following day. If the Gelatin Solution did not flow when inverted, the solution was deemed acceptable for ability to gel. No significant trend in gelation or liquification is observed in the timeframe tested.

In addition, for the frozen samples, viscosity of the liquefied gelatin solution was measured using a cone-and-plate viscometer at 37° C. and a shear rate of 150-250 s⁻¹. No significant trend in gelatin viscosity was observed in the timeframe tested.

As part of the refrigeration and freezing storage stability study, samples were tested for sterility (BacT/Alert). Tests were negative (no growth in 5 days) after 8 weeks refrigerated and 24 weeks frozen.

NKA Stability

Experiments were also conducted to demonstrate that NKA produced with human kidney donors can be stored at cold temperature (2-8° C.). NKA stability was established with measurement of viability, phenotypic characterization and cell function in the product.

SRC were obtained from kidney tissue biopsies from four kidney tissue samples and NKA were prepared using standard procedures. After end of manufacturing, NKA were held at cold temperature for up to 7 days to evaluate shelf life. Samples were taken at Day 1, 2, 3, 4 and 7 for analysis.

Stability of SRC Viability in NKA

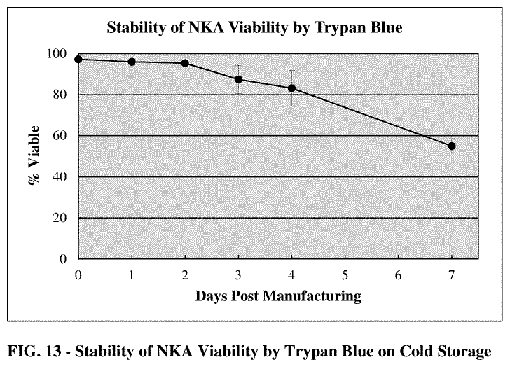

Viability of SRC in NKA was measured by Trypan Blue dye exclusion. FIG. 13 illustrates stability of SRC viability after the product had been store cold for up to 7 days post manufacturing. SRC viability remains above 70% (industry standard) for at least 4 days in cold storage.

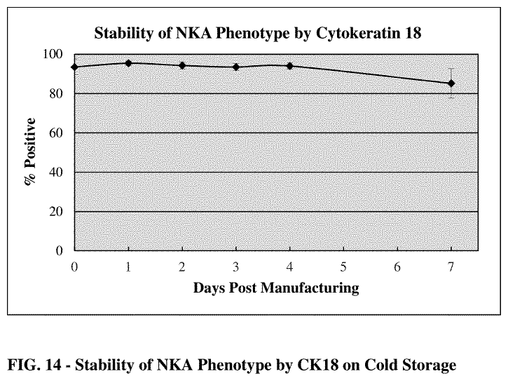

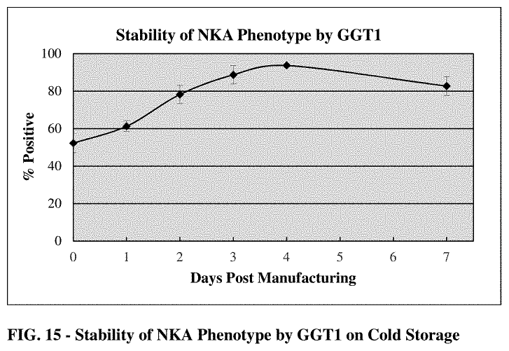

Stability of SRC Phenotype in NKA SRC Phenotype in NKA was measured by expression of CK18 and GGT1. FIGS. 14 and 15 illustrate stability of SRC phenotype after the product had been in cold storage for up to 7 days post manufacturing. SRC phenotype by CK18 and GGT1 remains above release criteria for at least 4 days in cold storage.

Stability of SRC Function in NKA

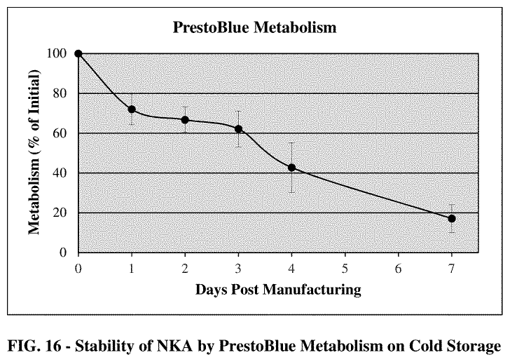

PrestoBlue metabolism and VEGF production were used as a measure of SRC function in the product. FIG. 16 illustrates PrestoBlue metabolism after the in cold storage for up to 7 days post manufacturing. The ability of SRC in NKA to metabolize PrestoBlue steadily declines with storage time as would be expected for cells stored without nutrition. At day 3 in cold storage NKA metabolism was greater than 50% of initial PrestoBlue value and meets proposed release criteria. A shelf life of 3 days is estimated based on SRC function on cold storage of NKA.

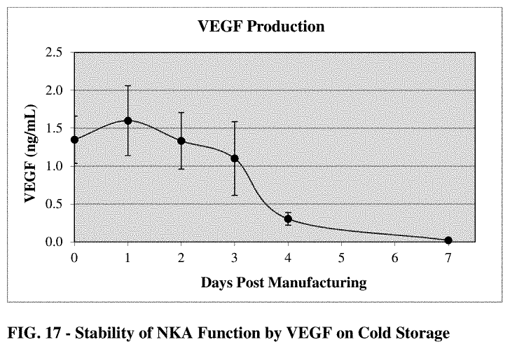

FIG. 17 illustrates VEGF production after the product had been in cold storage for up to 7 days post manufacturing. The ability of SRC in NKA to express VEGF is stable to day 3 (no statistical difference from day 0) and declines with further storage time as would be expected for cells stored without nutrition. At day 3 in cold storage VEGF production meets proposed release criteria. A shelf life of 3 days is estimated based on evaluation of SRC function during cold storage of NKA.

A shelf life of 3 days is placed on NKA based on maintenance of SRC viability at >70% at day 3 in storage. At Day 3 PrestoBlue metabolism as a measure of cell function is above 50% of initial value at Day 0. A decline in PrestoBlue metabolism is expected in cells stored without nutrients.

NKA can be stored for 3 days post-manufacturing at cold temperature based on maintenance of SRC viability at target level of 70%, and maintain cell phenotype and function that meet release specifications.

### Example 4: NKA Delivery and Implantation

NKA is targeted for injection into the kidney cortex of the patient using a cell delivery system. Components used in the delivery system and injection procedure are covered in the following sections.

NKA Delivery System

NKA delivery system is composed of a cannula (needle) compatible with cell delivery and a syringe. Different vendors use the terms cannula or needle to describe cell delivery products. For this description the terms trocar, cannula and needle are used interchangeably.

The main component of NKA delivery system is the delivery needle/cannula. Desirable features of the delivery cannula for effective delivery of NKA in the clinic are listed in Table 9. In addition, we will use a cannula that is compatible with NKA.

TABLE 9
Features of NKA Delivery Cannula

| Delivery | | |
|---|---|---|
| Cannula/ | | |
| Needle Feature | Target | Rationale |
| Shaft | 18-26 gauge | Smaller than a standard biopsy |
| | | tool (15 gauge) |
| Tip | Non-coring | Minimize recipient tissue damage |
| Hole size | ≥0.35 mm | Minimize cell damage |
| Hole placement | Side opening | Minimize product leakage |
| | holes | along needle shaft |
| Markings | Depth lines | Target delivery to location |

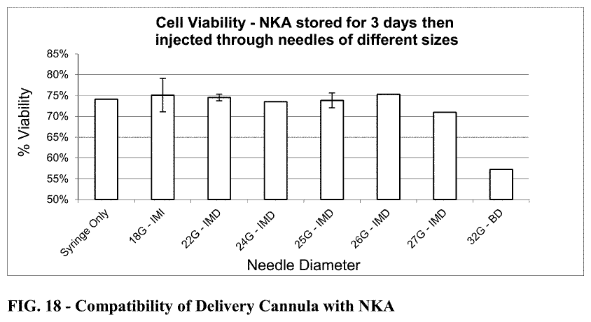

Syringe materials are compliant with USP Class VI guidelines and tested following ISO 10993 methods to assess biocompatibility. Syringes are sourced from Merit Medical, Becton Dickinson or similar vendors that meet biocompatibility classification and product compatibility testing. Delivery needles/cannula are procured from Cook Medical, Bloomington, Ind., International Medical Development, Huntsville, Utah, Innovative Med Inc., Irvine Calif. or similar that meet biocompatibility requirements and product compatibility testing. Product compatibility testing of 18-32 gauge delivery cannulas with NKA is shown in FIG. 18. SRC viability on passage through the cannula is the same as for the syringe alone for cannulas from 18 to 26 gauge demonstrating that these cannulas are compatible with the SRC. SRC viability seems to drop for needle sizes smaller than 26 gauge.

NKA Implantation

In preparation for implantation, NKA is warmed to room temperature just before injection into the kidney to liquefy the product.

NKA is targeted for implantation into the kidney cortex via a needle/cannula and syringe compatible with cell delivery. The intent is to introduce NKA via penetration of the kidney capsule and deposit into multiple sites of the kidney cortex. Initially, the kidney capsule will be pierced using a 15-20 gauge access trocar/cannula inserted approximately 1 cm into the kidney cortex (but not advanced further into the kidney). NKA will be contained in a syringe that will be attached to a blunt tipped inner cell delivery needle or flexible cannula (18-26 gauge, as suitable for the access cannula). In the Phase 1 clinical study, NKA was delivered via an 18G delivery needle. The proposed Phase II study will utilize an 18 gauge or smaller needle for cell delivery. The delivery needle will be threaded inside the access cannula and advanced into the kidney, into which the NKA will be administered. Injection of the NKA will be at a rate of 1-2 mL/min. After each 1-2 minute injection, the inner needle will be retracted along the needle course within the cortex to the second site of injection; and so forth until the needle tip is at the end of the access cannula or the entire cell volume has been injected. This system allows for both laparoscopic and percutaneous delivery. Under percutaneous delivery, the placement of the access cannula/trocar and delivery needle will be performed using direct, real-time image guidance. Injection of the NKA will be monitored with ultrasound image guidance to visualize the microbubble footprint of NKA deposits.

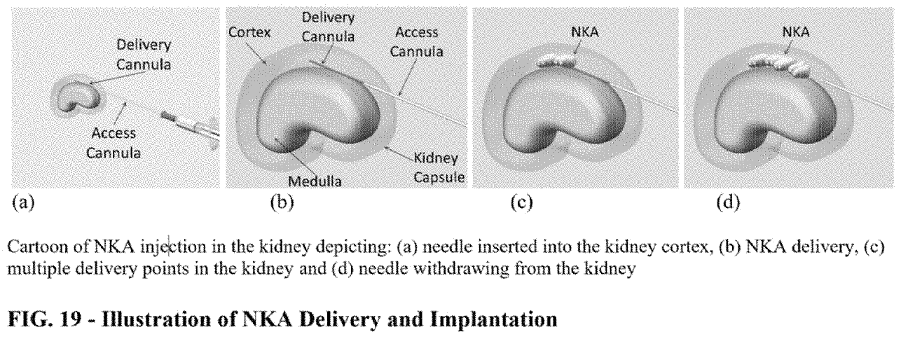

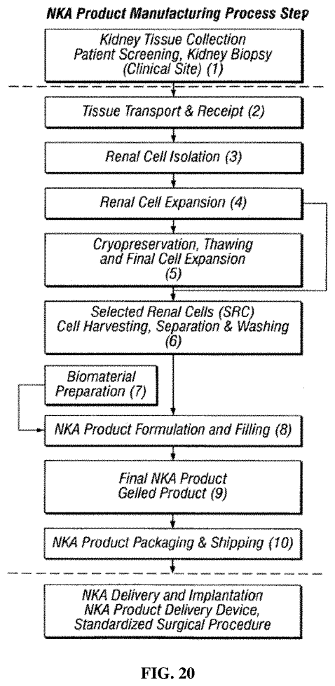

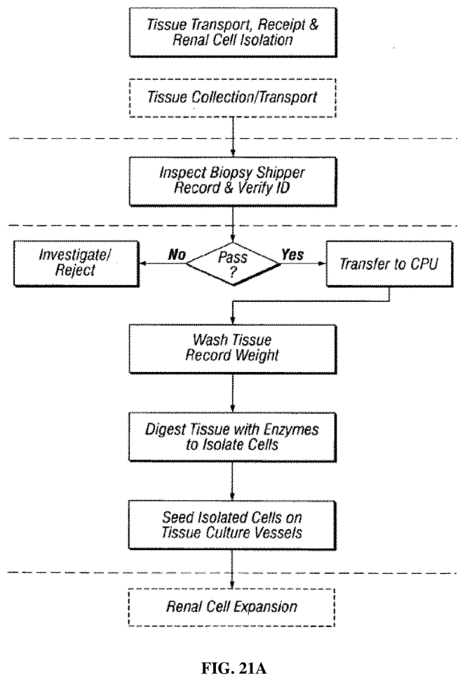

The schematic in FIG. 19 illustrates the concept of injecting NKA into a kidney using a needle compatible with cell delivery and distribution into a solid organ. NKA will be delivered directly into the kidney cortex. NKA delivery in patients will initially use a standardized percutaneous or laparoscopic procedure.

### Example 5: Non-limiting Examples of Methods and Compositions for Producing SRCs

### Example 5.1—Preparation of Solutions

This example section provides the compositions of the various media formulations and solutions used for the isolation and characterization of the heterogeneous renal cell population, and manufacture of the regenerative therapy product, in this example.

TABLE 10
Culture Media and Solutions

| Material | Composition |
|---|---|
| Tissue Transport Medium | Viaspan ™ or HypoThermosol-FRS ® |
| | or DMEM |
| | Kanamycin: 100 μg/mL |
| Renal Cell Growth Medium | DMEM:KSFM (50:50) |
| | 5% FBS |
| | Growth Supplements: |
| | HGF: 10 mg/L |
| | EGF: 2.5 μg/L |
| | Insulin: 10.0 mg/L, |
| | Transferrin: 5.5 mg/L |
| | Selenium: 670 μg/L |
| | Kanamycin: 100 μg/mL |
| Tissue Wash Solution | DMEM |
| | Kanamycin: 100 μg/mL |
| Digestion Solution | Collagenase IV: 300 Units |
| | Dispase: 5 mg/mL |
| | Calcium Chloride: 5 mM |
| Cell Dissociation Solution | TrypLE ™ |
| Density Gradient Solution | 7% OptiPrep |
| | OptiMEM |
| Cryopreservation Solution | DMEM or HypoThermosol-FRS |
| | 10% DMSO |
| | 10% FBS |

Dulbecco's Phosphate Buffered Saline (DPBS) was used for all cell washes.

### Example 5.2—Isolation of the Heterogeneous Unfractionated Renal Cell Population

This example section illustrates the isolation of an unfractionated (UNFX) heterogeneous renal cell population from human. Initial tissue dissociation was performed to generate heterogeneous cell suspensions from human kidney tissue.

Renal tissue via kidney biopsy provided the source material for a heterogeneous renal cell population. Renal tissue comprising one or more of cortical, corticomedullary junction or medullary tissue may be used. It is preferred that the corticomedullary junction tissue is used. Multiple biopsy cores (minimum 2), avoiding scar tissue, were required from a CKD kidney. Renal tissue was obtained by the clinical investigator from the patient at the clinical site approximately 4 weeks in advance of planned implantation of the final NKA. The tissue was transported in the Tissue Transport Medium of Example 5.1.

The tissue was then washed with Tissue Wash Solution of Example 5.1 in order to reduce incoming bioburden before processing the tissue for cell extractions.

Renal tissue was minced, weighed, and dissociated in the Digestion Solution of Example 5.1. The resulting cell suspension was neutralized in Dulbecco's Modified Eagle Medium (D-MEM)+10% fetal bovine serum (FBS) (Invitrogen, Carlsbad Calif.), washed, and resuspended in serum-free, supplement-free, Keratinocyte Media (KSFM) (Invitrogen). Cell suspensions were then centrifuged over a 15% (w/v) iodixanol (OptiPrep™, Sigma) density boundary to remove red blood cells and debris prior to initiation of culture onto tissue culture treated polystyrene flasks or dishes at a density of 25,000 cells per cm² in Renal Cell Growth Medium of Example 5.1. For example, cells may be plated onto T500 Nunc flask at 25×10⁶ cells/flask in 150 ml of 50:50 media.

### Example 5.3—Cell Expansion of the Isolated Renal Cell Population

Renal cell expansion is dependent on the amount of tissue received and on the success of isolating renal cells from the incoming tissue. Isolated cells can be cryopreserved, if required (see infra). Renal cell growth kinetics may vary from sample to sample due to the inherent variability of cells isolated from individual patients.

A defined cell expansion process was developed that accommodates the range of cell recoveries resulting from the variability of incoming tissue Table 11. Expansion of renal cells involves serial passages in closed culture vessels (e.g., T-flasks, Cell Factories, HyperStacks®) in Renal Cell Growth Medium Table 10 using defined cell culture procedures.

A BPE-free medium was developed for human clinical trials to eliminate the inherent risks associated with the use of BPE. Cell growth, phenotype (CK18) and cell function (GGT and LAP enzymatic activity) were evaluated in BPE-free medium and compared to BPE containing medium used in the animal studies. Renal cell growth, phenotype and function were equivalent in the two media. (data not shown)

TABLE 11
Cell Recovery from Human Kidney Biopsies

| | Renal cells | |
|---|---|---|
| | (cells/10 mg tissue) | |
| Source | (passage 0) | (passage 1) |
| Human Kidney Tissue | 1.44 − 10.80 × 10⁶ | 4.61 − 23.10 × 10⁷ |
| Samples (n = 7) | | |

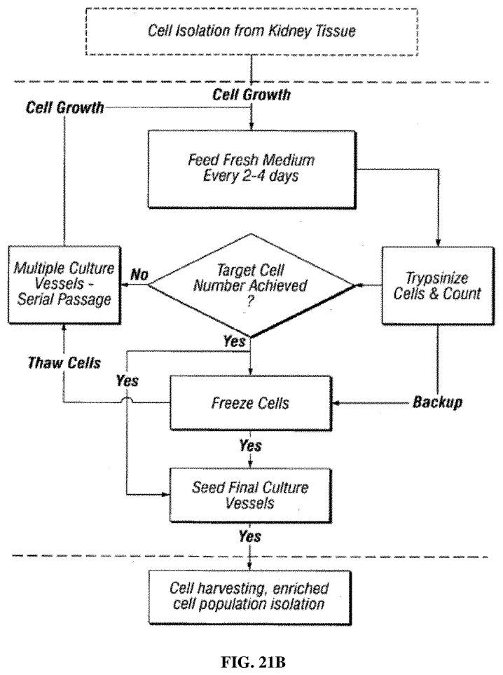

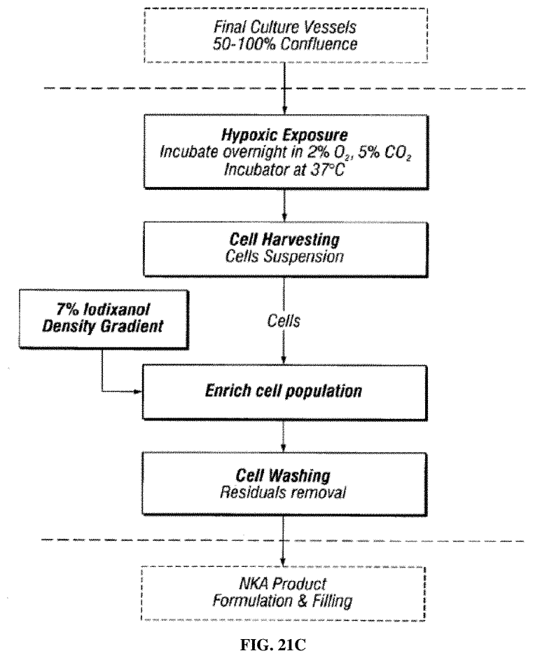

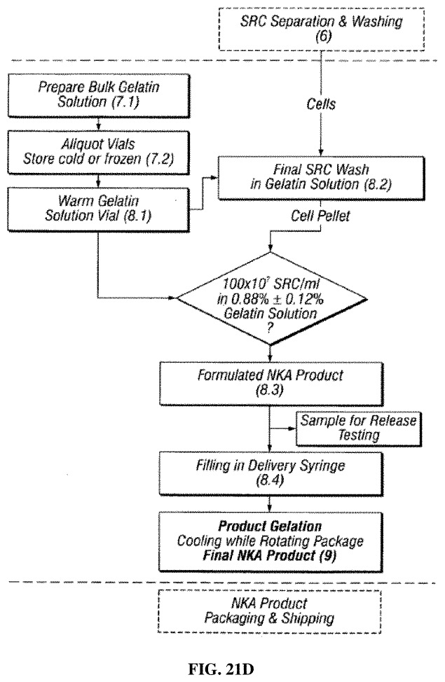

Once cell growth was observed in the initial T-flasks (passage 0) and there were no visual signs of contamination, culture medium was replaced and changed thereafter every 2-4 days (FIG. 21B). Cells were assessed to verify renal cell morphology by visual observation of cultures under the microscope. Cultures characteristically demonstrated a tight pavement or cobblestone appearance, due to the cells clustering together. These morphological characteristics vary during expansion and may not be present at every passage. Cell culture confluence was estimated using an Image Library of cells at various levels of confluence in the culture vessels employed throughout cell expansions.

Renal cells were passaged by trypsinization when culture vessels are at least 50% confluent (FIG. 21B). Detached cells were collected into vessels containing Renal Cell Growth Medium, counted and cell viability calculated. At each cell passage, cells were seeded at 500-4000 cells/cm² in a sufficient number of culture vessels in order to expand the cell number to that required for formulation of NKA (FIG. 21B). Culture vessels were placed in a 37° C. incubator in a 5% CO₂ environment. As described above, cell morphology and confluence was monitored and tissue culture media was replaced every 2-4 days. Table 12 lists the viability of human renal cells observed during cell isolation and expansion of six kidney biopsies from human donors.

TABLE 12
Cell Viability of Human Renal Cells in Culture

| Passage (n = 6) | Cell Viability (Average %) | Range (%) |
|---|---|---|
| P0 | 88 | 84-93 |
| P1 | 91 | 80-98 |
| P2 | 94 | 92-99 |
| P3 | 98 | 97-99 |

Inherent variability of tissue from different patients resulted in different cell yield in culture. Therefore, it is not practical to strictly define the timing of cell passages or number and type of culture vessels required at each passage to attain target cell numbers. Typically renal cells undergo 2 or 3 passages; however, duration of culture and cell yield can vary depending on the cell growth rate.

Cells were detached for harvest or passage with 0.25% Trypsin with EDTA (Invitrogen). Viability was assessed via Trypan Blue exclusion and enumeration was performed manually using a hemacytometer or using the automated Cellometer® counting system (Nexcelom Bioscience, Lawrence Mass.).

### Example 5.4 Cryopreservation of Cultured Cells

Expanded renal cells were routinely cryopreserved to accommodate for inherent variability of cell growth from individual patients and to deliver product on a pre-determined clinical schedule. Cryopreserved cells also provide a backup source of cells in the event that another NKA is needed (e.g., delay due to patient sickness, unforeseen process events, etc.). Conditions were established that have been used to cryopreserve cells and recover viable, functional cells upon thawing.

For cryopreservation, cells were suspended to a final concentration of about 50×10⁶ cells/mL in Cryopreservation Solution (see Example 5.1) and dispensed into vials. One ml vials containing about 50×10⁶ cells/mL were placed in the freezing chamber of a controlled rate freezer and frozen at a pre-programmed rate. After freezing, the cells were transferred to a liquid nitrogen freezer for in-process storage.

### Example 5.5 Preparation of SRC Cell Population

Selected Renal Cells (SRC) can be prepared from the final culture vessels that are grown from cryopreserved cells or directly from expansion cultures depending on scheduling (FIG. 21B).

If using cryopreserved cells, the cells were thawed and plated on tissue culture vessels for one final expansion step. When the final culture vessels were approximately 50-100% confluent cells were ready for processing for SRC separation. Media exchanges and final washes of NKA dilute any residual Cryopreservation Solution in the final product.

Once the final cell culture vessels have reached at least 50% confluence the culture vessels were transferred to a hypoxic incubator set for 2% oxygen in a 5% CO₂ environment at 37° C. (FIG. 21C). and cultured overnight. Cells may be held in the oxygen-controlled incubator set to 2% oxygen for as long as 48 hours. Exposure to the more physiologically relevant low-oxygen (2%) environment improved cell separation efficiency and enabled greater detection of hypoxia-induced markers such as VEGF.

After the cells have been exposed to the hypoxic conditions for a sufficient time (e.g., overnight to 48 hours), the cells were detached with 0.25% Trypsin with EDTA (Invitrogen). Viability was assessed via Trypan Blue exclusion and enumeration was performed manually using a hemacytometer or using the automated Cellometer® counting system (Nexcelom Bioscience, Lawrence Mass.). Cells were washed once with DPBS and resuspended to about 850×10⁶ cells/mL in DPBS.

Centrifugation across a density boundary/interface was used to separate harvested renal cell populations based on cell buoyant density. Renal cell suspensions were separated by centrifugation over a 7% iodixanol Solution (OptiPrep; 60% (w/v) in OptiMEM; see Example 5.1).

The 7% OptiPrep density interface solution was prepared and refractive index indicative of desired density was measured (R.I. 1.3456+/−0.0004) prior to use. Harvested renal cells were layered on top of the solution. The density interface was centrifuged at 800 g for 20 min at room temperature (without brake) in either centrifuge tubes or a cell processor (e.g., COBE 2991). The cellular fraction exhibiting buoyant density greater than approximately 1.045 g/mL was collected after centrifugation as a distinct pellet. Cells maintaining a buoyant density of less than 1.045 g/mL were excluded and discarded.

The SRC pellet was re-suspended in DPBS (FIG. 21C). The carry-over of residual OptiPrep, FBS, culture medium and ancillary materials in the final product is minimized by 4 DPBS wash and 1 Gelatin Solution steps.

1. An injectable formulation comprising:
a) a temperature-sensitive cell-stabilizing biomaterial, and
b) a bioactive renal cell (BRC) population,
wherein the temperature-sensitive cell-stabilizing biomaterial is a hydrogel that
(i) maintains a substantially solid state at about 8° C. or below, wherein the substantially solid state is a gel state,
(ii) maintains a substantially liquid state at about ambient temperature or above, and
(iii) has a solid-to-liquid transitional state between about 8° C. and about ambient temperature or above,
wherein the hydrogel comprises an extracellular matrix protein of recombinant origin, is derived from extracellular matrix sourced from kidney or another tissue or organ, or comprises gelatin.

2. The injectable formulation of claim 1, wherein the gelatin is derived from Type I, alpha I collagen.

3-5. (canceled)

6. The injectable formulation of claim 1, wherein the BRC is a selected renal cell (SRC) population.

7. (canceled)

8. The injectable formulation of claim 6, wherein the BRC or SRC population is enriched for tubular renal cells.

9-10. (canceled)

11. The injectable formulation of claim 8, wherein the SRC population is characterized by phenotypic expression of one or more tubular epithelial cell markers, wherein the one or more tubular epithelial cell markers comprise CK18 and/or GGT1.

12-13. (canceled)

14. The injectable formulation of claim 8, wherein the SRC population is characterized by phenotypic expression of one or more viability and/or functionality markers, and wherein the one or more viability and/or functionality markers comprise VEGF and/or KIM-1.

15. The injectable formulation of claim 8, wherein the SRC population is characterized by LAP and/or GGT enzymatic activity.

16-20. (canceled)

21. An implantable formulation comprising:
a) a decellularized kidney of human or animal origin or a cell-stabilizing biomaterial that has been structurally engineered through three dimensional bioprinting, and
b) a bioactive renal cell (BRC) population.

22. (canceled)

23. The injectable formulation of claim 1, wherein:
a) the biomaterial comprises gelatin at about 0.88% (w/v), and wherein the gelatin is derived from Type I, alpha I collagen, and
b) the BRC population comprises a selected renal cell (SRC) population, wherein the SRC population comprises an enriched population of tubular renal cells and has a density greater than about 1.04 g/mL.

24. A method for preparing an injectable formulation comprising a temperature-sensitive cell-stabilizing biomaterial and an admixture of bioactive renal cells, the method comprising the steps of: i) obtaining renal cortical tissue from the donor/recipient; ii) isolating renal cells from the kidney tissue by enzymatic digestion and expanding the renal cells using standard cell culture techniques; iii) subjecting the harvested renal cells to separation across a density boundary or density interface or single step discontinuous gradient to obtain an SRC population; and iv) reconstituting the bioactive cells with a gelatin-based hydrogel biomaterial, wherein the gelatin is derived from Type I, alpha I collagen.

25. The method of claim 24, wherein the selected renal cells comprise an enriched population of tubular renal cells and having a density greater than about 1.04 g/mL.

26. The method of claim 24, wherein the harvested renal cells are exposed to hypoxic culture conditions prior to separation across a density boundary or density interface or continuous or discontinuous single step or multistep density gradient.

27. The method of claim 24, wherein the renal cells are enriched for tubular renal cells.

28-32. (canceled)

33. The method of claim 24, further comprising monitoring the renal cells for phenotypic expression of one or more viability and/or functionality markers, wherein the one or more viability and/or functionality markers comprise VEGF and/or KIM-1.

34. (canceled)

35. The method of claim 24, further comprising monitoring the renal cells for phenotypic expression of one or more tubular epithelial cell markers, wherein the one or more tubular epithelial cell markers comprise CK18 and/or GGT1.

36. (canceled)

37. The method of claim 24, further comprising monitoring renal cell functionality by measuring enzymatic activity, wherein the measured enzymatic activity is for LAP and/or GGT.

38-40. (canceled)

41. The method of claim 24, wherein the SRC are resuspended in a liquefied gelatin solution at 26-30° C.

42. The method of claim 41, wherein the SRC are resuspended in sufficient gelatin solution to achieve an SRC concentration of 100×10⁶ cells/ml.

43. The method of claim 24, further comprising rapidly cooling the SRC/gelatin solution to stabilize the biomaterial such that the SRC will remain suspended in the gel on storage.

44-47. (canceled)

48. A method of treating kidney disease in a subject, the method comprising injecting the formulation of claim 1 into the subject, wherein the formulation is injected through a 18 to 30 gauge needle.

49. (canceled)
